(19) United States
(12) Patent Application Publication
(10) Pub. No.: US 2013/0131146 A1
(43) Pub. Date: May 23, 2013

(54) Compositions and Methods for Treating Pulmonary Conditions

(71) Applicants: Neeraj Vij, Baltimore, MD (US); Manish Bodas, Baltimore, MD (US)

(72) Inventors: Neeraj Vij, Baltimore, MD (US); Manish Bodas, Baltimore, MD (US)

(73) Assignee: THE JOHNS HOPKINS UNIVERSITY, Baltimore, MD (US)

(21) Appl. No.: 13/695,155

(22) PCT Filed: May 2, 2011
(86) PCT No.: PCT/US11/34769

§ 371 Date: Feb. 6, 2013

Related U.S. Application Data
(60) Provisional application No. 61/329,791, filed on Apr. 30, 2010.

(51) Int. Cl. A61K 31/7105; C12Q 1/28; G01N 33/68

(52) U.S. Cl. CPC A61K 31/7105; G01N 33/68; C12Q 1/28
USPC 514/44 A; 435/7.21; 436/501; 435/28

(57) ABSTRACT

The present invention relates to the role of cystic fibrosis transmembrane conductance regulator (CFTR) in pulmonary conditions. In one embodiment, a method for assessing the severity of lung damage from a pulmonary condition in a subject comprises the steps of (a) measuring the level and/or functional activity of membrane/lipid-raft cystic fibrosis transmembrane conductance regulator (CFTR)in a sample from the subject; (b) measuring the level of ceramide or its species in a sample from the subject; and (c) comparing the membrane/lipid- raft CFTR level and/or functional activity and ceramide level to a control sample, wherein a difference in membrane/lipid-raft CFTR level and/or functional activity and ceramide level is indicative of the severity of lung damage. The method can further comprise treating the subject based on the severity of lung damage. In particular embodiments, the treatment comprises administering a CFTR agonist and/or an agent that inhibits the synthesis of ccramide or its species.

### CROSS-REFERENCE TO RELATED APPLICATIONS

This application claims the benefit of U.S. Provisional Application No. 61/329,791, filed Apr. 30, 2010, which is incorporated herein by reference in its entirety.

### STATEMENT OF GOVERNMENTAL INTEREST

This invention was made with U.S. government support under grant no. NNJ06HI17G, grant no. CTSA UL RR025005, and grant no. CTSA UL RHL096931. The U.S. government has certain rights in the invention.

### FIELD OF THE INVENTION

The present invention relates to the field of pulmonary conditions. More specifically, the present invention relates to the role of cystic fibrosis transmembrane conductance regulator (CFTR) in pulmonary conditions.

### BACKGROUND OF THE INVENTION

Chronic obstructive pulmonary disease (COPD), emphysema, asthma, and cystic fibrosis (CF) subjects suffer from severe tissue-debilitating lung inflammation that is induced by exposure to environmental contaminants like cigarette smoke (CS) and bacterial infections (1-3). In addition, the Pseudomonas aeruginosa bacterial infection has been shown to have a critical role in pathogenesis of both CF and COPD (4-6), but it is not clear why these patients are highly sensitive to P. aeruginosa (PA) infections. Accordingly, there is a great need to understand the abnormal metabolic pathways in patients with such pulmonary conditions, and to identify new methods for diagnosis and treatment prognosis, as well as new means of therapeutic intervention.

### SUMMARY OF THE INVENTION

The present invention relates to the field of pulmonary conditions. More specifically, the present invention relates to the role of cystic fibrosis trans-membrane conductance regulator (CFTR) in pulmonary conditions. The present invention is based, in part, on the discovery that expression of membrane and lipid-raft localized CFTR correlates with ceramide signaling and severity of lung disease. The inventors have demonstrated that CFTR regulates tight junction formation, ceramide accumulation and inflammatory signaling in pulmonary conditions. In addition, the critical role of membrane/lipid-raft CFTR in regulating cigarette smoke induced apoptosis and autophagy during lung injury has also been identified.

Accordingly, in one aspect, lipid-raft CFTR expression can be targeted as a potent therapeutic strategy for controlling ceramide elicited pulmonary conditions. In particular embodiments, the therapeutic strategy involves the use of agents to inhibit the synthesis of ceramide or its species (e.g., Lactosylceramide (LacCer)). The methods of the present invention can be used to inhibit synthesis of ceramide or its species by inhibiting one or more of the enzymes involved in the ceramide synthesis pathways including the de novo, sphingomyelin, and salvage pathways, or an enzyme involved in the synthesis of a ceramide species. The methods also involve the administration of an effective amount of a CFTR agonist. In another aspect, the present invention provides methods for determining severity of lung disease that determine prognosis-based therapeutic strategies.

Accordingly, in certain embodiments, a method for assessing the severity of lung damage from a pulmonary condition in a subject comprises the steps of (a) measuring the level and/or functional activity of membrane/lipid-raft cystic fibrosis transmembrane conductance regulator (CFTR) in a sample from the subject; (b) measuring the level of ceramide or its species in a sample from the subject; and (c) comparing the membrane/lipid-raft CFTR level and/or functional activity and level of ceramide or its species to a control sample, wherein a difference in membrane/lipid-raft CFTR level and/or functional activity and level of ceramide or its species is indicative of the severity of lung damage. The method can further comprise treating the subject based on the severity of lung damage. In particular embodiments, the treatment comprises administering an effective amount of an agent that inhibits the synthesis of ceramide or its species. In an additional or alternative embodiment, the treatment comprises administering an effective amount of a CFTR agonist.

The present invention also provides a method for predicting risk of lung damage from a pulmonary condition in a subject comprising the steps of (a) measuring the level and/or functional activity of membrane/lipid-raft CFTR in a sample from the subject; (b) measuring the level of ceramide or its species in a sample from the subject; and (c) comparing the membrane/lipid-raft CFTR level and/or functional activity and level of ceramide or its species to a control sample, wherein a difference in membrane/lipid-raft CFTR level and/or functional activity and level of ceramide or its species is indicative of a risk of lung damage. The method can further comprise treating the subject based on the risk of lung damage. In particular embodiments, the treatment comprises administering an effective amount of an agent that inhibits the synthesis of ceramide or its species. The treatment can also comprise administering an effective amount of a CFTR agonist.

In another embodiment, the present invention provides a method for treating a pulmonary condition in a subject comprising the steps of (a) measuring the level and/or functional activity of membrane/lipid-raft CFTR in a sample from the subject; (b) measuring the level of ceramide or its species in a sample from the subject; (c) comparing the membrane/lipid-raft CFTR level and/or functional activity and level of ceramide or its species to a control sample; and (d) administering an effective amount of a ceramide inhibitor and/or a CFTR agonist based on the membrane/lipid-raft CFTR level and/or functional activity and level of ceramide or its species. In certain embodiments, the ceramide inhibitor inhibits one or more of the enzymes involved in the synthesis of ceramide or its species.

In an alternative embodiment, a method for treating a pulmonary condition in a subject comprises the step of administering a therapeutically effective amount of at least one agent that inhibits the synthesis of ceramide or its species. In one embodiment, the at least one agent is an antisense molecule. In another embodiment, the at least one agent is an RNA interference agent. In yet a further embodiment, the at least one agent is an miRNA. In yet a further embodiment, a method for treating a pulmonary condition in a subject comprises the step of administering a therapeutically effective amount of at least one CFTR agonist that increases membrane/lipid-raft level and/or functional activity.

In several embodiments, the pulmonary condition is selected from the group consisting of chronic obstructive pulmonary disease (COPD), emphysema, cystic fibrosis, Pseudomonas aeruginosa bacterial infection, or biomass/cigarette-smoke exposure. In a specific embodiment, the pulmonary condition is COPD. In another embodiment, the pulmonary condition is emphysema. In yet another embodiment, the pulmonary condition is cystic fibrosis. In yet another embodiment, the subject is a smoker or is exposed to biomass smoke. In particular embodiments, the ceramide species is lactosylceramide.

The enzyme that inhibits the synthesis of ceramide can be selected from the group consisting of serine palmitoyltransferase, 3-ketosphinganine reductase, dihydroceramide synthase, dihydroceramide desaturase, sphingomyelinase, ceramide synthase, and lactosylceramide synthase.

### BRIEF DESCRIPTION OF THE FIGURES

FIG. 1 shows that CFTR regulates innate and adaptive immune responses. In FIG. 1A and FIG. 1B, the macrophages and neutrophils isolated from Cftr^{-/-} mice show significant increase in constitutive (FIG. 1A) IL-6 and (FIG. 1B) MPO (myeloperoxidase levels, only in neutrophils) secretion in the culture supernatants compared with that of the Cftr^{+/+}. ***p<0.001. In FIG. 1C, the BALF from Cftr^{-/-} mice show significant increase in the basal and Pa-LPS (20 μg i.t., 24 h) induced MPO levels compared with those of the Cftr^{+/+}. *p<0.05. In FIG. 1D, the splenocytes from Cftr^{-/-} mice show significantly higher Con A (5 or 10 μg/ml) induced cell proliferation compared with that of the Cftr^{+/+} mice. **p<0.01; ***p<0.001. In FIG. 1E, the culture supernatants from the splenocytes of FIG. 1D have significantly higher IL-6 levels in the Cftr^{-/-} compared with those of the Cftr^{+/+}. ***p<0.001. In FIG. 1F, the flow cytometry analysis shows Cftr expression in CD4+ Cftr^{+/+} mice splenocytes (i), and Cftr^{-/-} splenocytes were used as a negative control. The significant increase in percentage of CD4⁺IFN-γ⁺ (ii) and CD4⁺Foxp3⁺ (iii) cells in the Cftr^{-/-} splenocytes compared with that of the Cftr^{-/-} is indicative of the constitutive T cell activation in the absence of CFTR. In FIG. 1G, immunofluorescence staining verifies the increase in constitutive and Pa-LPS-induced Foxp3 expression (primary-rabbit polyclonal, secondary-goat antirabbit IgG-FITC) and nuclear localization in Cftr^{-/-} mice lungs compared with that of the Cftr^{+/+}. Original magnification 320; scale bar, 50 μm. In FIG. 1H, differences in basal and Con A (5 μg/ml) induced Foxp3 expression in Cftr^{+/+} and Cftr^{-/-}splenocytes is confirmed by Western blotting. β-actin blot shows the equal loading. In FIG. 1I, densitometry analysis of Foxp3 expression (in FIG. 1H) normalized to β-actin. Data represent n=3 in each group, and error bars depict mean±SEM.

FIG. 2 demonstrates that ceramide and ZO-1 expression is elevated in immune cells of Cftr^{-/-} mice. Flow cytometry analysis showing ZO-1 and ceramide expression in macrophages (FIG. 2A) and neutrophils (FIG. 2B) from Cftr^{+/+} and Cftr^{-/-} mice. Thioglycolate-elicited peritoneal macrophages and neutrophils were immunostained for Mac-3 (macrophage) and NIMP-R14 (neutrophil) markers, and co-staining with ceramide (left panels) or ZO-1 (right panels) Abs was used to quantify the percentage changes in the number of positive cells. The upper right quadrants show the percentage gated cells positive for both the primary Abs as indicated. Data from n=3 mice show a very significant increase in ceramide-positive cells in Cftr^{-/-} mice (97.85%) derived macrophages compared with that of the Cftr^{+/+} mice (0.99%) (FIG. 2A, left panel), whereas neutrophils (FIG. 2B, left panel) have no change. In contrast, expression of lipid-raft marker ZO-1 shows a significant increase in both the cell types (FIGS. A and B, right panels) in the absence of CFTR (Cftr^{-/-}) indicating the role of CFTR in tight junction formation.

FIG. 3 illustrates that the severity of inflammatory lung disease inversely correlates with the membrane-CFTR levels. In FIG. 3A, human lung tissue sections from each group at Gold stage 0 (at risk), I (mild), II (moderate), and III-IV (severe and very severe) COPD (n=4 to 10) were stained with H&E (bottom row) showing a significant increase in inflammatory cells and emphysema in moderate and severe COPD compared with that in mild COPD. The lung tissue sections immunostained with CFTR (green, top row) or ceramide (green, third row) show significant decrease in membrane CFTR expression at advanced stage of COPD lung disease while ceramide levels increase. Nuclear (Hoechst) staining is shown in blue (second and fourth rows). Original magnification 320 and 363; scale bars: white, 50 μm; red, 10 μm; black, 100 μm. In FIG. 3B, densitometric analysis confirms the statistical significance (p<0.001) and illustrates the correlation of CFTR and ceramide expression with severity of lung emphysema. In FIG. 3C, the HEK-293 cells transfected with WT-CFTR and treated with increasing doses of CSE for 12 h (n=3) show an inverse relationship between increasing CSE dose and expression of membrane CFTR (mature C band, left panel). The total cell lysates from HEK-293 cells, either control (a) or transfected with WT-CFTR (b), show the absence of CFTR (B and C bands) in the control cells (right panel). In FIG. 3D, the lung lysates from air and CS exposed mice (n=3) were used for either Cftr immunoprecipitation (CFTR-169, upper panel) or lipid-raft isolation, and CFTR protein levels were detected by Western blotting. The data show a significant decrease in membrane and lipid-raft CFTR protein expression in the lungs of CS-exposed mice. In FIG. 3E, densitometry analysis of membrane and raft CFTR expression from control and CS groups (in FIG. 3D) is shown as mean±SEM of triplicate samples. **p<0.01; ***p<0.001. In FIG. 3F, the longitudinal lung sections from air or CS exposed mice (same experiment as FIG. 3D) show an increased ceramide and ZO-1 co-staining (red arrow) in the CS-exposed lungs verifying that CS modulates lipid-raft and ceramide signaling in murine lungs.

FIG. 4 shows that CFTR regulates de novo and membrane ceramide signaling. BALF from three to five C57BL/6 Cftr^{+/+} or Cftr^{-/-} mice, treated intratracheally with PBS (control), Pa-LPS (20 μg/mouse; 12 h), FB1 (50 μg/mouse; 24 h) and/or AMT (50 mg/mouse; 24 h) was used to quantify the IL-6 and IL-10 levels. In FIG. 4A, inhibition of de novo ceramide synthesis by FB1 treatment significantly decreases Pa-LPS-induced IL-6 and IL-10 in Cftr^{+/+} mice (i, ii), but FB1 has a modest effect on Pa-LPS-induced IL-6 levels in Cftr^{-/-} mice (iii, iv). In FIG. 4B, inhibition of membrane-ceramide release by AMT treatment is relatively less protective against Pa-LPS-induced lung injury in Cftr^{+/+} mice (i, ii) but effectively controls the inflammatory cytokines in Cftr^{-/-} mice (iii, iv). The data show that inhibition of de novo and membrane-ceramide release can control Pa-LPS-induced lung injury in the presence or absence of CFTR, respectively. This also indicates that CFTR can regulate de novo and membrane-ceramide signaling. Data represent the averages of triplicate ELISAs from n=3 to 5 samples and are shown as mean±SEM. *p<0.05; **p<0.01; ***p<0.001.

FIG. 5 demonstrates that CFTR regulates lipid-raft expression and signaling via ceramide. In FIG. 5A, CFBE41o-WT-CFTR (WT-CFBE) and CFBE41o-cells were stimulated with Pa-LPS (10 ng/ml) or FB1 (50 μM) for 24 h. The lipid-raft protein extracts were isolated from these cells, and expression of lipid-raft marker ZO-2 was quantified by Western blotting. Data show significant downregulation (>2-fold) of lipid-raft ZO-2 expression with Pa-LPS or FB1 treatment only in the presence of WT-CFTR indicating that CFTR is a critical regulator of Pa-LPS or ceramide mediated lipid-raft expression and signaling. The same membrane was blotted with α-actin as a loading control. In FIG. 5B, immunostaining for ZO-2 shows its increased expression in lung tissue sections from Cftr^{-/-} mice (n=3) compared with that of the Cftr^{+/+}(n=3) (top panels) verifying that CFTR regulates the expression of lipid-raft protein, ZO-2. Nuclear staining is shown in blue (bottom panels). In FIG. 5C, the lung sections from Cftr^{+/+} and Cftr^{-/-} mice (n=4-5), treated with PBS or Pa-LPS (20 mg/mouse; 24 h), immunostained for ZO-1 (green, goat anti-rabbit IgG FITC) and ceramide (red, donkey anti-mouse Dylight 594), show significant increase in constitutive and Pa-LPS-induced ZO-1 and ceramide levels (top row) in Cftr^{-/-} compared with those in Cftr^{+/+}. The colocalization of ceramide with ZO-1 verifies the lipid-raft localization of ceramide in the absence of CFTR. The CFTR immunostaining (green, third row, goat anti-rabbit IgG FITC) shows the CFTR expression levels in the Cftr^{+/+} mice lungs, and Cftr^{-/-} are shown as a negative control. Nuclear (Hoechst) staining is shown in blue (second and fourth rows) and H&E staining shows increase in constitutive and Pa-LPS induced inflammation (bottom row). Original magnification 320; scale bars: white, 50 μm; red, 10 μm; black, 100 μm. In FIG. 5D, the densitometry and Spearman's correlation coefficient analysis of ZO-1 and ceramide staining (FIG. 5C) shows the statistical significance of immunostaining data.

FIG. 6 shows that the PDZ-interacting domain of CFTR regulates ceramide accumulation. In FIG. 6A, the HEK-293 cells were transiently transfected with pEGFP WT-CFTR or ΔTRL-CFTR plasmid constructs, and one experimental group was treated with 100 μg/ml CSE for 12 h. The cells were stained and analyzed for ceramide (R-PE, FL-2) and GFP expression (FL-1) by flow cytometry. The data represent three independent experiments. Expression of CFTR lacking the PDZ-interacting domain shows an increase in basal (49.85-56.48%) and CSE-induced ceramide accumulation (69.22-80.56%), indicating the crucial role of PDZ binding domains in regulating CFTR-dependent ceramide signaling. In FIG. 6B, the HEK-293 cells transiently overexpressing WT-CFTR or ΔTRLCFTR plasmids (n=3) were incubated with FITC-labeled E. coli LPS for 3 h and analyzed by flow cytometry (unpermeabilized cells). The transient expression of CFTR lacking the PDZ binding domain results in reduced binding of LPS to the plasma membrane. It is anticipated that less LPS binding to ΔTRL expressing cells is a direct consequence of its reduced cell surface expression and/or lipid-raft translocation. In FIG. 6C, the lipid-raft proteins from HEK-293 cells expressing WT-CFTR or ΔTRL-CFTR were analyzed for CFTR expression by Western blotting (a, 30-s exposure; b, 20-min exposure). The data show that lack of the PDZ-interacting domain of CFTR compromises its membrane expression (b, left panel) and translocation to the lipid-rafts (a and b, right panel). FIG. 6D shows the densitometry analysis of membrane- and raft-CFTR expression from WT-CFTR and ΔTRL-CFTR groups in FIG. 6C.

FIG. 7. Schematic of CFTR-mediated ceramide signaling. Schematic illustrates the critical role of lipid-raft CFTR in controlling ceramide (sphingomyelin) and inflammatory (TNF-a) or apoptotic (CD95) signaling. Our model predicts that the absence or decrease in lipid-raft CFTR expression culminates these regulatory functions, resulting in NF-kB-mediated hyperinflammatory response. Environmental factors such as P. aeruginosa infection or CS exposure further exaggerate the lipid-raft signaling and contribute to the pathogenesis of chronic inflammatory or apoptotic signaling by modulating CFTR lipid-raft expression that controls ceramide accumulation. We anticipate that in the absence of lipid-raft CFTR, membrane-ceramide accumulation induces lipid-raft fusion and large-scale clustering of the membrane receptors that result in lung injury and emphysema.

FIG. 8 demonstrates that the inhibition of de novo ceramide synthesis controls Pa-LPS induced lung injury in Cftr^{+/+} mice. The paraffin embedded longitudinal lung sections from 3-5 C57BL/6 Cftr^{+/+} or Cftr^{-/-} mice, treated intratracheally with PBS (Control), Pa-LPS (20 μg/mouse; 12 hrs) and/or Fumonisin B1 (FB1, 50 μg/mouse; 24 hrs) were immunostained for ceramide, NFκB and neutrophils (NIMP-R14). The co-staining with the Hoechst dye was used to localize the nucleus (blue) Inhibition of de novo ceramide synthesis by FB1 treatment shows the significant decrease in Pa-LPS induced ceramide accumulation, NFκB levels and neutrophil infiltration (green) in the Cftr^{+/+} mice. The representative sections show that FB1 treatment is relatively less effective in controlling Pa-LPS induced lung injury in Cftr^{-/-} mice. The H&E staining shows the morphology and inflammatory state of the representative lung section. The data suggest that inhibition of de novo ceramide synthesis by FB1 can effectively control Pa-LPS induced lung injury in the presence of CFTR. Scale: white bar-50 μm, red bar-10 μm, black-100 μm.

FIG. 9 shows that inhibition of membrane ceramide synthesis controls Pa-LPS induced lung injury in Cftr^{-/-} mice. The paraffin embedded longitudinal lung sections from 3-5 C57BL/6 Cftr^{+/+} or Cftr^{-/-} mice, treated intratracheally with PBS (Control), Pa-LPS (20 μg/mouse; 12 hrs) and/or Amitriptyline (AMT, 50 μg/mouse; 24 hrs) were immunostained for ceramide, NFκB and neutrophils (NIMP-R14). The co-staining with the Hoechst dye was used to localize the nucleus (blue). Inhibition of membrane ceramide synthesis by AMT treatment shows the significant decrease in Pa-LPS induced ceramide accumulation, NFκB levels and neutrophil infiltration (green) in the Cftr^{-/-} mice. The representative sections show that AMT treatment is relatively less effective in controlling Pa-LPS induced lung injury in the Cftr^{+/+} mice. The H&E staining shows the morphology and inflammatory state of the representative lung section. The data suggest that inhibition of membrane ceramide synthesis by AMT is effective in controlling Pa-LPS induced lung injury in the absence of CFTR. Scale: white bar-50 μm, red bar-10 μm, black-100 μm).

FIG. 10 shows that lipid-raft CFTR regulates ceramide signaling. In FIG. 10A, CFBE41o- and CFBE4lo-WT-CTFR cells were treated with 10 ng/ml Pa-LPS±5 mM CD (methyl-(3-cyclodextrin) for 6 hrs and stained for NFκB (green) or Hoechst (nucleus, blue). CFBE41o-cells show constitutively elevated NFκB activity (right panel, white arrow) that is downregulated in cells stably transduced with WT-CFTR (left panel). CD treatment depletes CFTR from lipid-rafts and abrogates its control of NFκB signaling (left panel); (magnification: 64×, n=3). In FIG. 10B(i), ceramide (red) or Hoechst (nucleus, blue) staining of CFBE41o- and CFBE4lo-WT-CFTR cells show that CFBE41O-cells have constitutively elevated ceramide levels that is downregulated in cells stably transduced with WT-CFTR (magnification: 64×, n=3). In FIG. 10B(i), the quantitative analysis of IL-8 secretion by ELISA shows that depleting CFTR from cholesterol rich lipid-rafts by 5 mM CD treatment for 24 hours significantly induces (mean±SEM, n=3 p<0.001) IL-8 chemokine levels. In FIG. 10C, Cftr^{+/+} C57BL/6 mice (n=3) were treated intratracheally (i.t) with methyl-β-cyclodextrin (CD, 75 μg/mouse for 3 days). The immunofluorescence staining show that depleting Cftr from cholesterol rich lipid-rafts (CFTR/ZO-2) by CD treatment (right panel as compared to untreated left panel) induces NFκB activity and neutrophil chemotaxis (NIMPR14). A significant increase was also observed in expression of lipid-raft marker (ZO-2) by CD treatment indicative of elevated lipid-raft expression and clustering. Nuclear staining (Hoechst) for the representative pictures is shown in blue (Scale: white bar-50 μm). In FIG. 10D, CFBE4lo-wt-CFTR and CFBE4lo-cells were transiently transfected with NFκB reporter construct and a renila luciferase internal control plasmid. The cells were induced with 10 ng/ml TNFα and/or treated with 50 μM Fumonisin B1 (FB1) for 12 hours. The relative NFκB promoter activity (%, n=3) normalized to renila luciferase internal control is shown. TNFα induced NFκB promoter driven luciferase expression, and inhibition of de novo ceramide synthesis by FB1 inhibits TNFα mediated NFκB activity (*p<0.05) only in the presence of cell surface CFTR (CFBE41o-wt-CFTR).

FIG. 11 demonstrates that lipid-raft CFTR expression controls membrane ceramide accumulation. The CFBE4lo-WT-CTFR cells were treated with PBS (control, FIG. 10A), methyl-β-cyclodextrin (CD, 5 mM, FIG. 10B) and/or TNFα (10 ng/ml, FIG. 10C) for 6 hrs. The co-immunostaining and confocal imaging for ZO-1 (green) and ceramide (red) shows the changes in membrane ceramide levels. Z stacking of representative confocal images is shown in the upper panel. Data indicates that lipid-raft CFTR depletion by CD treatment increases ceramide accumulation on the cell surface (z stack) and lipid rafts (arrow). The induction of CFTR translocation to lipid-rafts by TNFα depletes CD induced membrane ceramide accumulation (bottom panel). The representative data from n=3 samples is shown at 40× magnification. Scale: x=0.44 μm, y=0.44 μm and z=2 μm.

FIG. 12 shows that CFTR controls cigarette smoke (CS) induced apoptosis in murine lungs. In FIG. 12A, the paraffin embedded longitudinal lung sections from Cftr^{+/+} or Cftr^{-/-} (n=3) mice exposed to room-air or CS were used to detect the number of apoptotic cells by a TUNEL assay (upper panel). Inset: A higher magnification of the image of CS-exposed Cftr^{-/-} mice lung section showing alveolar type II TUNEL positive cells (red arrows). The co-staining with Hoechst dye (middle panel) was used to localize the nucleus (blue). The data shows significant increase in constitutive and CS induced TUNEL-positive apoptotic cells (p<0.01, lower panel) in the Cftr^{-/-} mice compared to Cftr^{+/+} (lower panel). In FIGS. 12B and 12 C, the longitudinal lung sections from the same groups of mice were immunostained for Fas- and ceramide-expression (upper panels). The co-staining with Hoechst dye (middle panels) was used to localize the nucleus (blue). The representative sections (12B and 12C) show that absence of CFTR triggers significant increase in Fas- and ceramide-expression in the murine lungs that is further upregulated by CS exposure (p<0.005, lower panel). Scale: white bar-50 μm. The data indicate that CFTR regulates CS induced apoptosis in the murine lungs.

FIG. 13 illustrates that defective CFTR increases the expression of lipid-raft proteins. In FIGS. 13A and 13B, the longitudinal lung sections from Cftr^{+/+} or Cftr^{-/-} (n=3) mice exposed to room-air or CS were immunostained for ZO-1 (FIG. 13A, upper panel) and ZO-2 (FIG. 13B, upper panel). The co-staining with Hoechst dye (middle panel) was used to localize the nucleus (blue, middle panels). The expression of these lipid-raft proteins (ZO-1/2) is significantly (lower panel) elevated in the absence of CFTR that is further increased by CS exposure (p<0.001, Scale: white bar-50 μm). In FIG. 13C, the western blot analysis of total lung lysates from Cftr^{+/+} or Cftr^{-/-} mice exposed to room air or CS shows upregulation of ZO-1 expression in the absence of CFTR. β-actin was used as a loading control. The data implies that CFTR regulates the expression of lipid-raft proteins thereby modulating the clustering of signaling receptors.

In FIG. 14, the absence of CFTR worsens CS induced inflammation and apoptotic cell death. In FIG. 14A, the H&E staining of longitudinal lung sections from Cftr^{+/+} or Cftr^{-/-} mice (n=3) exposed to room-air or CS were used to detect the inflammatory milieu in the lungs. The data indicate that the absence of CFTR triggers a higher constitutive as well as CS induced inflammation in the murine lungs, Scale: black bar-50 μm. In FIG. 14B, the NFκB immunostaining (upper panel) of these mice lung sections demonstrate that presence of CFTR (Cftr^{+/+}-mice) significantly (lower panel) inhibits NFκB activation and nuclear translocation (inset, upper panel) in acute CS induced lung injury (p<0.01, bottom panel, Scale: white bar—50 μm, red bar—10 μm). The co-staining with Hoechst dye (middle panel) was used to localize the nucleus (blue). In FIG. 14C, the total lung lysates of Cftr^{+/+} and Cftr^{-/-} mice show a significant increase (p<0.05, mean±SEM) in caspase-3/7 activity in the absence of CFTR. In FIG. 14D, the immunoblotting of lipid-raft protein fraction of murine (Cftr^{+/+}, n=3) lungs from cyclodextrin (CD, at indicated time points, scale) treated and/or sub-chronic CS exposed mice show an increase in Fas levels while raft-CFTR (r-CFTR) expression decreases. The α-actin was used as a loading control. In FIGS. 14E and 14F, the immunoblotting of lipid-raft fractions of CFBE41o- and CFBE41o-WT-CFTR cells shows the correlation of stable lipid-raft WT-CFTR expression (FIG. 14E) with a significant (p<0.001, mean±SEM) decrease in caspase-3/7 activity (FIG. 14F). The data implies that CFTR controls CS induced lung injury and apoptosis.

FIG. 15 shows that WT-CFTR controls Fas expression and apoptosis. In FIG. 15A, the western blot analysis of total protein lysates from HEK-293 cells transiently transfected with control (p-EGFP) or WTCFTR-GFP plasmids show significant decrease (p<0.05, mean±SEM, right panel) in Fas expression in the presence of WT-CFTR. β-actin was used as a loading control. In FIG. 15B, the flow cytometry of HEK-293 cells transiently transfected with WT-CFTR-GFP plasmid±CSE (200 μg/ml) shows that CSE treatment significantly decreases (p<0.001, mean±SD) WT-CFTR expression (bottom panel). In FIG. 15C, the propidium iodide (PI) staining of HEK-293 cells transiently transfected with control (p-EGFP) or WT-CFTR-GFP plasmids±CSE shows that WT-CFTR controls CSE induced apoptosis as seen by decrease in number of cells in M1-phase (bottom panel). The data demonstrates that over-expression of WT-CFTR controls Fas expression and CSE induced apoptosis.

FIG. 16 indicates that the absence of CFTR triggers CS induced aberrant autophagy response. In FIG. 16A, the longitudinal lung sections of Cftr^{+/+} and Cftr^{-/-} mice exposed to room-air or CS were immunostained for p62 (upper panel). The co-staining with the Hoechst dye (middle panel) was used to localize the nucleus (blue). The data indicate a significant (lower panel) increase in p62 expression (p<0.01) and peri-nuclear localization (inset, upper right panel) in the absence of CFTR (constitutive and post-CS exposure). In FIG. 16B, the western blot analysis of total protein lysates from Cftr^{+/+} and Cftr^{-/-} mice (upper panel) demonstrates a significant constitutive increase in p62 protein levels in the Cftr^{-/-} mice lungs compared to Cftr^{+/+} (p=0.04, lower panel, mean±SEM). In FIG. 16C, the data shows that the intrinsically higher p62 protein expression in CFBE41o-cells can be significantly downregulated by stable WT-CFTR expression (CFBE41o-WT-CTFR cells) (p<0.05, lower panel, mean±SEM). Treatment with CSE further induces p62 levels in CFBE41o-, although the increase was much lower in the presence of WT-CFTR (p<0.05, lower panel). In FIG. 16D, the flow cytometry analysis of HEK-293 cells shows that WT-CFTR expression significantly controls CSE induced in p62-positive cells (p<0.001, mean±SD). The percentage of p62-positive cells is shown as the sum of upper right and upper left quadrants). In FIG. 16E, the CFBE41o-cells show significant (p<0.001, right panel) increase in peri-nuclear accumulation of autophagy marker, LC3-GFP (inset, left panel) compared to 16HBEo-cells (n=3) containing normal membrane-CFTR levels. The data also shows further significant (p<0.005, right panel) increase in CSE induced LC3-GFP peri-nuclear aggregates (inset) in absence of membrane-CFTR (CFBE41o-). Scale: white bar-50 μm, red bar-10 μM. The data implies that WT-CFTR is required for protective autophagy response in acute CS mediated lung injury.

FIG. 17 is a schematic showing the regulation of CS induced lung injury by lipid-raft CFTR. The schematic illustrates the critical role of lipid-raft CFTR in controlling apoptotic (CD95/Fas) and autophagy response to cigarette smoke (CS) induced lung injury. The model predicts that the CS mediated decrease in lipid-raft CFTR expression modulates these responses via NFκB/Fas resulting in increased apoptosis and aberrant-autophagy. Environmental factors such as CS exposure may modulate the lipid-raft clustering by controlling CFTR lipid-raft expression that regulates membrane-ceramide accumulation. CS mediated decrease in lipid-raft CFTR expression may induce membrane-ceramide accumulation that results in lipid-raft fusion and large scale clustering of the membrane receptors (like Fas) as a mechanism for pathogenesis of chronic lung injury and emphysema.

FIG. 18 shows that CFTR controls alveolar cell death and ceramide accumulation. In FIG. 18A, the TUNEL staining (upper panel) of the longitudinal lung sections of Cftr^{+/+} and Cftr^{-/-} mice (n=3) indicate a significant constitutive (lower panel, p<0.001) increase in the number of TUNEL-positive apoptotic cells in the absence of CFTR that is further increased by Pa-LPS induced lung injury (lower panel, p<0.05). The co-staining with the Hoechst dye (middle panel) was used to localize the nucleus (blue, middle panel). In FIG. 18B, higher magnification of TUNEL staining of LPS- and CS-treated Cftr^{-/-} lung section shows alveolar type II cells (red arrow) as the predominant cell type involved in LPS or CS induced lung injury in the absence of CFTR. Scale: white bar-50 μm. In FIG. 18C, the dot blot of lipid-raft fractions isolated from Cftr^{+/+} and Cftr^{-/-} mice lungs show an increase in lipid-raft ceramide-accumulation in the absence of CFTR. The data confirms that membrane-CFTR controls alveolar cell apoptosis and ceramide accumulation.

FIG. 19 demonstrates that CFTR regulates ceramide accumulation, apoptosis and autophagy. In FIG. 19A, the co-immunostaining of ceramide (red) and ZO-1 (green) in longitudinal lung sections from CS exposed Cftr^{+/+} and Cftr^{-/-} mice indicate substantial increase in ceramide-ZO-1 co-localization (yellow) in the absence of CFTR. The lung sections were also co-stained with Hoechst dye to localize the nucleus (blue). The data demonstrate that in CS induced lung injury, ceramide accumulates in the ZO-1+ lipid-rafts that may facilitate the clustering of lipid-raft signaling platforms. Scale: white bar-50 μm. In FIG. 19B, the fold change (mean±SEM) in caspase-3/7 activities of CFBE41o- and CFBE41-wtCFTR cells treated with either DMSO (control) or CSE shows a significant decrease in constitutive (p<0.001, 1.6 fold) and CSE induced (p<0.0001, 2.6 fold) caspase-3/7 activity in the presence of WT-CFTR (CFBE41o-wtCFTR cells). In FIG. 19C, the longitudinal lung sections from acute CS-exposed Cftr^{+/+} and Cftr^{-/-} mice show higher perinuclear accumulation of LC30 (autophagy marker) in the CS-exposed Cftr^{-/-} mice. Scale: red bar-10 μM. The data implies that lipid-raft CFTR controls CS induced membrane-ceramide accumulation, apoptosis and autophagy response.

FIG. 20 shows the effect of CFTR-deficiency or cigarette smoke on lipid-raft and total protein loading controls. In FIG. 20A, the western blot analysis of total protein lysates from CFBE41o- and CFBE41-wtCFTR cells shows equal α/β-actin expression, suggesting that CFTR deficiency does not alter the expression of common lipid-raft loading control (α-actin). In FIG. 20B, the expression of α-actin in the total protein lysates from air and CS-exposed Cftr^{+/+} and Cftr^{-/-} mice is comparable to β-actin indicating that expression of common lipid-raft loading control is not altered by CFTR-deficiency or CS exposure.

FIG. 21 shows that increased LacCer- and p62-accumulation correlates with severity of emphysema. In FIG. 21A, the human lung tissue sections from non-emphysema controls [(GOLD 0, n=5, smokers (Smk)] and COPD subjects (GOLD I-IV) with emphysema [(n=36, smokers (Smk)] were immunostained with LacCer, a sphingolipid, or p62, defective-autophagy marker (upper panel). The data implies a significant positive correlation (lower panels, densitometry analysis, p<0.01) between increased LacCer- and p62-accumulation with severity of emphysema in COPD subjects. The nuclear staining (Hoechst) in the bottom panel shows the selected tissue area. In FIG. 21B, the co-immunolocalization of LacCer with lipid-raft marker ZO-2 in the lung tissue sections (n=60) demonstrate that LacCer-accumulation in lipid-rafts increases with severity of emphysema in COPD subjects. In FIG. 21C, the lipid-raft proteins isolated from human lung tissue from non-emphysema control (GOLD 0) and emphysema-COPD (GOLD II and IV) subjects were used to verify the significant increase (lower panel, densitometric analysis, p<0.05) in raft-LacCer accumulation with severity of emphysema (n=3). The α-actin was used as a loading control. In FIG. 21D, immunoblotting of total protein lysates from lung tissues of non-emphysema control (GOLD 0) and emphysema-COPD (GOLD IV) subjects show a significant increase in p62 (defective-autophagy), p53 (senescence) and CHOP (apoptosis) expression (n=4) in the COPD subjects compared to control (p<0.05). Scale: white bar-50 μm, red bar-10 μm.

FIG. 22 shows that inhibition of LacCer-synthase ameliorates chronic-CS induced lung inflammation and emphysema. In FIG. 22A, the longitudinal lung tissue sections from C57BL/6 mice exposed to air- or chronic-CS (Ch-CS, 24 weeks) and/or treated with LacCer-synthase inhibitor, D-PDMP (50 μg/mouse, see scale for treatment regime, n=4-5), stained with H&E show a significant decrease in CS induced inflammation and emphysema on treatment with D-PDMP (upper panel & b, p=0.0016). The immunostaining of these lung tissue sections shows a considerable increase in LacCer-accumulation upon chronic-CS-exposure (middle panel) that is significantly downregulated by D-PDMP (p<0.05). The nuclear staining (Hoechst) in the bottom panel shows the selected tissue area and the densitometric analysis of the data is shown in the right panel. In FIG. 22B, the morphometric analysis of lung tissue sections stained with H&E (FIG. 21A) demonstrate a significant increase in alveolar diameter upon chronic-CS exposure (p=0.000017). This alveolar space enlargement was significantly controlled by pharmacological inhibition of LacCer-synthase (D-PDMP, p=0.0016). In FIG. 22C, the chronic-CS exposed mice show a significant increase in BALF IL-6 levels (p=0.01) that can be rescued to basal levels by treatment with DPDMP (n=3, p=0.009). In FIG. 22D, the flow cytometric analysis of bronchoalveolar fluid (BALF)-cells isolated from chronic-CS exposed and/or D-PDMP treated mice show that D-PDMP treatment significantly controls (p<0.01) chronic-CS induced macrophage (F4/80-positive cells) infiltration. Scale: white/black bar-50 μm.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### I. DEFINITIONS

“Agent” refers to any and all materials that may be used as or in pharmaceutical compositions, including any and all materials such as small synthetic or naturally derived organic compounds, nucleic acids, polypeptides, antibodies, fragments, isoforms, variants, or other materials that may be used independently for such purposes, all in accordance with the present invention.

The term “agonist” refers to an agent that up-regulates (e.g., stimulates or increases) at least one bioactivity of a protein. An agonist may be an agent which promotes or increases the interaction between a protein and another molecule, e.g., a target peptide or enzyme substrate. An agonist may also be an agent that up-regulates expression of a gene or which increases the amount of expressed protein present. In particular embodiments, an “agonist” refers to an agent that increases the level and/or functional activity of membrane/lipid-raft CFTR.

As used herein, and unless otherwise indicated, the term “antisense oligonucleotide” refers to an oligonucleotide having a sequence complementary to a target DNA or RNA sequence.

As used herein, the term “antisense strand” of an siRNA or RNAi agent e.g., an antisense strand of an siRNA duplex or siRNA sequence, refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific RNA interference (RNAi), e.g., complementarity sufficient to trigger the destruction of the desired target mRNA by the RNAi machinery or process. The term “sense strand” or “second strand” of a siRNA or RNAi agent e.g., an antisense strand of an siRNA duplex or siRNA sequence, refers to a strand that is complementary to the antisense strand or first strand. Antisense and sense strands can also be referred to as first or second strands, the first or second strand having complementarity to the target sequence and the respective second or first strand having complementarity to said first or second strand.

The term “CFTR” as used herein means cystic fibrosis transmembrane conductance regulator or a mutation thereof capable of regulator activity, including, but not limited to, AF508 CFTR and G551D CFTR (see, e.g., http://www.genet.sickkids.on.ca/cftr/, for CFTR mutations).

As used herein, “comparing” in relation to “the proportion, level, or cellular localization, to a standard proportion, level, or cellular localization” refers to making an assessment of the how the proportion, level, or cellular localization of a CFTR transcript or protein in a sample relates to the proportion, level, or cellular localization of a CFTR transcript or protein of the standard. For example, assessing whether the proportion, level, or cellular localization of the CFTR transcript or protein of the sample is the same as, more or less than, or different from the proportion, level, or cellular localization CFTR transcript or protein of the standard or control.

As used herein, and unless otherwise indicated, the term “complementary,” when used to describe a sequence in relation to a target sequence, means that the sequence is able to bind to the target sequence in a cellular environment in a manner sufficient to disrupt the function (e.g., replication, splicing, transcription or translation) of the gene comprising the target sequence. The binding may result from interactions such as, but not limited to, nucleotide base parings (e.g., A-T/G-C). In particular embodiments of the invention, a sequence is complementary when it hybridizes to its target sequence under high stringency, e.g., conditions for hybridization and washing under which nucleotide sequences, which are at least 60 percent (preferably greater than about 70, 80, or 90 percent) identical to each other, typically remain hybridized to each other. Such stringent conditions are known to those skilled in the art, and can be found, for example, in Current Protocols in Molecular Biology, John Wiley & Sons, N.Y. (1989), 6.3.1-6.3.6, which is incorporated herein by reference. Another example of stringent hybridization conditions is hybridization of the nucleotide sequences in 6× sodium chloride/sodium citrate (SSC) at about 45° C., followed by 0.2×SSC, 0.1% SDS at 50-65° C. Particularly preferred stringency conditions are hybridization in 6× sodium chloride/sodium citrate (SSC) at about 45° C., followed by one or more washes in 0.2.×SSC, 0.1% SDS at 50° C. Depending on the conditions under which binding sufficient to disrupt the functions of a gene occurs, a sequence complementary to a target sequence within the gene need not be 100 percent identical to the target sequence. For example, a sequence can be complementary to its target sequence when at least about 70, 80, 90, or 95 percent of its nucleotides bind via matched base pairings with nucleotides of the target sequence. As used herein, the term “correlating” can be used in reference to a parameter, e.g., a proportion, level, or cellular localization in a cell from a subject. “Correlating” as used according to the present invention may be by any method of relating levels of expression, activity and/or localization of a biomarker(s) (e.g., CFTR) and/or ceramide-LacCer levels to a standard valuable for the assessment of the diagnosis, prediction of pulmonary condition progression, assessment of efficacy of clinical treatment, selection of a subject for a particular treatment, monitoring of the progress of treatment with a particular therapy, and in the context of a screening assay, for the identification of an agent that inhibits a ceramide synthesis enzyme.

When used to describe the sequences of siRNAs, the term “corresponding to,” as used herein, means that a siRNA has a sequence that is identical or complementary to the portion of target mRNA that is transcribed from the denoted DNA sequence.

As used herein, and unless otherwise indicated, the term “inhibiting the synthesis or expression” of a gene means impeding, slowing or preventing one or more steps by which the end-product protein encoded by said gene is synthesized. Typically, the inhibition involves blocking of one or more steps in the gene's replication, transcription, splicing or translation through a mechanism that comprises recognition of a target site located within the gene or transcript sequence based on sequence complementation. In a specific embodiment, inhibition of a ceramide synthesis enzyme reduces the amount of enzyme in the cell by greater than about 20%, 40%, 60%, 80%, 85%, 90%, 95%, or 100%. The amount of a ceramide synthesis enzyme can be determined by well-known methods including, but are not limited to, densitometer, fluorometer, radiography, luminometer, antibody-based methods and activity measurements.

The term “measuring” means methods which include detecting the presence or absence of a biomarker(s) in a sample, quantifying the amount of biomarker(s) in the sample, and/or qualifying the type of biomarker(s). Measuring can be accomplished by methods known in the art and those further described herein including, but not limited to, immunoassay.

A “patient,” “subject,” or “host” to be treated by the present methods refers to either a human or non-human animal, such as primates, mammals, and vertebrates. In particular, the terms refer to a human.

The terms “pulmonary condition”, “lung injury”, and “lung disease” are used interchangeably here and refer to both infection/smoke- and non-infection/smoke-induced disease and dysfunction of the respiratory system. The terms generally include any CFTR-mediated condition or symptom. A CFTR-mediated condition or symptom means any condition, disorder or disease, or symptom of such condition, disorder, or disease, that results from activity of cystic fibrosis transmembrane conductance regulator protein (CFTR). Non-limiting examples of pulmonary conditions include genetic conditions, acquired conditions (environmental exposure like biomass/cigarette smoke, etc.), primary conditions, secondary conditions, asthma, chronic obstructive pulmonary disease (COPD), cystic fibrosis, bronchiolitis, pneumonia, bronchitis, emphysema, adult respiratory distress syndrome, allergies, lung cancer, small cell lung cancer, primary lung cancer, metastatic lung cancer, brochiectasis, bronchopulmonary dysplasia, chronic bronchitis, chronic lower respiratory diseases, croup, high altitude pulmonary edema, pulmonary fibrosis, interstitial lung disease, reactive airway disease, lymphangioleiomyomatosis, neonatal respiratory distress syndrome, parainfluenza, pleural effusion, pleurisy, pneumothorax, primary pulmonary hypertension, psittacosis, pulmonary edema secondary to various causes, pulmonary embolism, pulmonary hypertension secondary to various causes, respiratory failure secondary to various causes, sleep apnea, sarcoidosis, smoking, stridor, acute respiratory distress syndrome, infectious diseases, SARS, tuberculosis, psittacosis infection, Q fever, parainfluenza, respiratory syncytial virus, combinations thereof, and conditions caused by any one or combination of the above. In particular embodiments, the terms “pulmonary condition”, “lung injury”, and “lung disease” refer to disease and/or conditions caused, directly or indirectly, implicating, or otherwise involving CFTR, particularly, those conditions correlated with lipid-raft CFTR expression and ceramide signaling.

The term “RNAi agent” or “RNAi-inducing entity” refers to an RNA species (other than a naturally occurring molecule not modified by the hand of man or transported into its location by the hand of man) whose presence within a cell results in RNAi and leads to reduced expression of an RNA to which the RNAi agent is targeted. The RNAi agent may be, for example, an siRNA or shRNA. In certain embodiments, an siRNA may contain a strand that inhibits expression of a target RNA via a translational repression pathway utilized by endogenous small RNAs referred to as microRNAs. In certain embodiments, an shRNA may be processed intracellularly to generate an siRNA that inhibits expression of a target RNA via this microRNA translational repression pathway. Any “target RNA” may be referred to as a “target transcript” regardless of whether the target RNA is a messenger RNA. The terms “target RNA” and “target transcript” are used interchangeably herein. The term RNAi agent or RNAi-inducing agent encompasses RNAi agents and vectors (other than naturally occurring molecules not modified by the hand of man as described above) whose presence within a cell results in RNAi and leads to reduced expression of a transcript to which the RNAi agent is targeted. At the level of post-transcriptional control, entirely new mechanisms of gene regulation have been discovered, typified by a large and growing class of ˜22-nucleotide-long non-coding RNAs, known as microRNAs (miRNAs), which function as repressors in all known genomes. The term “RNAi agent” or “RNAi-inducing entity” also encompasses such miRNAs.

The term “sample,” as used herein, refers to a biological sample obtained for the purpose of evaluation in vitro. In the methods of the present invention, the sample or patient sample may comprise any body fluid including, but not limited to, blood, serum, plasma, urine, saliva, and synovial fluid. A sample may also comprise any cells, tissue samples or cell components (such as cellular membranes or cellular components) obtained from a patient including a tissue biopsy.

An RNAi agent having a strand which is “sequence sufficiently complementary to a target mRNA sequence to direct target-specific RNA interference (RNAi)” means that the strand has a sequence sufficient to trigger the destruction of the target mRNA by the RNAi machinery or process.

A “small molecule” refers to a composition that has a molecular weight of less than 3 about kilodaltons (kDa), less than about 1.5 kilodaltons, or less than about 1 kilodalton. Small molecules may be nucleic acids, peptides, polypeptides, peptidomimetics, carbohydrates, lipids or other organic (carbon-containing) or inorganic molecules. A “small organic molecule” is an organic compound (or organic compound complexed with an inorganic compound (e.g., metal)) that has a molecular weight of less than about 3 kilodaltons, less than about 1.5 kilodaltons, or less than about 1 kDa.

Various methodologies of the instant invention include step that involves comparing a value, level, feature, characteristic, property, etc. to a “suitable control”, referred to interchangeably herein as an “appropriate control”. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing an RNAi methodology, for example, as described herein. In one embodiment, a transcription rate, mRNA level, translation rate, protein level, biological activity, cellular characteristic or property, genotype, phenotype, etc. can be determined prior to introducing a RNAi agent (siRNA, miRNA, etc.) of the invention into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

A “target gene” is a gene whose expression is to be selectively inhibited or “silenced”, e.g., a ceramide synthesis enzyme. In certain embodiments, this silencing is achieved by cleaving the mRNA of the target gene by a siRNA/miRNA that is created from an engineered RNA precursor by a cell's RNAi system or non-coding RNAs. One portion or segment of a duplex stem of the RNA precursor is an anti-sense strand that is complementary, e.g., fully complementary, to a section of about 18 to about 40 or more nucleotides of the mRNA of the target gene.

As used herein, the terms “treatment,” “treating,” “treat” and the like, refer to obtaining a desired pharmacologic and/or physiologic effect. The terms are also used in the context of the administration of a “therapeutically effective amount” of an agent, e.g., a ceramide inhibitor or a CFTR agonist. The effect may be prophylactic in terms of completely or partially preventing a particular outcome, disease or symptom thereof and/or may be therapeutic in terms of a partial or complete cure for a disease and/or adverse affect attributable to the disease. “Treatment,” as used herein, covers any treatment of a disease or condition in a subject, particularly in a human, and includes: (a) preventing the disease or condition from occurring in a subject which may be predisposed to the disease or condition but has not yet been diagnosed as having it; (b) inhibiting the disease or condition, i.e., arresting its development; and (c) relieving the disease or condition, e.g., causing regression of the disease or condition, e.g., to completely or partially remove symptoms of the disease or condition. In particular embodiments, the term is used in the context of treating a subject with a pulmonary condition.

### II. CFTR AGONISTS

In particular embodiments, the present invention provides CFTR agonists useful in the treatment of subject with pulmonary conditions. CFTR agonists are agents that stimulate or increase the level and/or functional activity of membrane/lipid-raft CFTR. The term also includes compounds that have activity in addition to CFTR agonistic activity. CFTR agonists includes agents, as defined herein (small synthetic or naturally derived organic compounds, nucleic acids, polypeptides, antibodies, fragments, isoforms, variants, etc.), as well as CFTR potentiators and CFTR corrector drugs including certain flavonoids, Cystic fibrosis foundation Therapeutics (CFFT) compounds (http://www.cftrfolding.org/CFFTReagents.htm) including CFTR targeted drugs from Vertex Pharmaceuticals. There are several known agents that could serve as CFTR agonists including, but not limited to, Quercetin, isoproterenol, GlyH-101, forskolin, IBMX, and apigenin. See also, U.S. Patent Applications Publication No. 20110098311, Publication No. 20110071206, Publication No. 20110008259, Publication No. 2010/02278888, Publication No. 20090143381, and Publication No. 2008/0146669. See also Sheppard et al., 18(2) CHEM. BIOL. 145-47 (2011); Pyle et al., 43 AM. J. RESP. CELL. MOL. BIOL. 607-16 (2010); Robert et al., 73(2) MOL. PHARM. 478-89 (2008); Yoo et al., 18(8) BIOORGANIC & MED. CHEM. LETTERS 2610-14 (2008); Pedemonte et al., 67 MOL. PHARM. 1797-1807 (2005); Yang et al., 278(37) J. BIOL. CHEM. 35079-85 (2003)

### III. CERAMIDE INHIBITORS

In one aspect, the present invention comprises the administration of an effective amount of a ceramide inhibitor. The term “ceramide inhibitor” refers to an agent, as defined herein, that inihibits the synthesis of ceramide, as well as ceramide metabolites or species including, but not limited to, lactosylceramide synthase (catalyzes the formation of the ceramide species, lactosylceramide (LacCer). Thus, in specific embodiments, a ceramide inhibitor of the present invention inhibits biosynthesis of ceramide or it species. More particularly, a ceramide inhibitor blocks one or more of the enzymes in the ceramide synthesis pathway (or synthesis of ceramide species). Ceramide synthesis in the body occurs via one of three major pathways. In the de novo pathway, ceramide is synthesized from less complex molecules in the body. The spingomyelin pathway produces ceramide through the breakdown of sphingomyelin mediated by the enzyme sphingomyelinase. In the salvage pathway, ceramide is produced by the breakdown of complex sphingolipids into sphingosine, which is then used to form ceramide.

The ceramide de novo pathway includes a series of enzymes that produce ceramide from the starting components serine and palmitoyl CoA. Serine palmitoyltransferase (SPT) catalyzes the first step in the synthesis of ceramide in the de novo pathway, which is the production of 3-ketodihydrosphingosine from serine and palmitoyl CoA. SPT inhibitors may include, but are not limited to, sphingo-fungins, lipoxamycin, myriocin, L-cycloserine and beta-chloro-L-alanine, as well as the class of Viridiofungins. Moreover, ceramide can be converted into LacCer by the enzyme LacCer synthase.

Another enzyme in the de novo pathway that may be targeted by a ceramide inhibitor is ceramide synthase (CerS). CerS catalyzes the acylation of the amino group of sphingosine, sphinganine, and other sphingoid bases using acyl CoA esters. CerS inhibitors may include, but are not limited to, the Fumonisins, the related AAL-toxin, and australifungins. The Fumonisin family of inhibitors is produced by Fusarium verticillioides and includes Fumonisin B1 (FB1). The N-acylated forms of FB1 are known to be potent CerS inhibitors while the O-deacylated form is less potent. Of the N-acylated forms of FB1, the erythro-, threo-2-amino-3-hydroxy-, and stereoisomers of 2-amino-3,5-dihydroxyoctadecanes are also known as CerS inhibitors. Australifungins from the organism Sporomiella australlis is also a potent inhibitor of CerS.

Dihydroceramide desaturase (DES) is the last enzyme in the de novo biosynthesis pathway of ceramide synthesis. At least two different forms, DES1 and DES2, are known. DES inhibitors may include, but are not limited to, the cyclopropene-containing sphingolipid GT11, as well as a-ketoamide (GT85, GT98, GT99), urea (GT55), and thiourea (GT77) analogs of this molecule.

Sphingomyelin hydrolysis by sphingomyelinases (SMases) produces phosphorylcholine and ceramide. At least five isotypes of SMase are known, including acid and neutral forms. Several physiological inhibitors of acid SMase have been described including amitriptyline, L-alpha-phosphatidyl-D-myo-inositol-3,5-bisphosphate, a specific acid SMase inhibitor, and L-alpha-phosphatidyl-D-myo-inositol-3,4,5-triphosphate, a non-competitive inhibitor of acid SMase. Ceramide-1-phosphate and sphingosine-1-phosphate have also been described as physiological inhibitors. Glutathione is an inhibitor of neutral SMase at physiological concentrations with a greater than 95% inhibition observed at 5 mM GSH. Compounds that are structurally unrelated to sphingomyelin but function as SMase inhibitors included desipramine, imipramine, SR33557, (3-carbazol-9-yl-propyl)-[2-(3,4-dimethoxy-phenyl)-ethyl)-methyl-amine (NB6), hexanoic acid (2-cyclo-pent-1-enyl-2-hydroxy-1-hydroxy-methyl-ethyl)-amide (NB12) CllAG, and GW4869. Compound SR33557 is a specific acid SMase inhibitor (72% inhibition at 30 μM). The compound NB6 has been reported as an inhibitor of the SMase gene transcription Inhibitors derived from natural sources include Scyphostatin, Macquarimicin A, and Alutenusin, which are non-competitive inhibitors of neutral SMase, and Chlorogentisylquinone, and Manumycin A, which are irreversible specific inhibitors of neutral SMase. Also described is a-Mangostin, an inhibitor of acid SMase. Scyphostatin analogs with inhibitory proprieties include spiroepoxide 1, Scyphostatin, and Manumycin A sphingolactones. Sphingomyelin analogs with inhibitory proprieties include 3-O-methylsphingomyelin, and 3-O-ethylsphingomyelin.

Other exemplary sphingomyelinase inhibitors known in the art include, but are not limited to, [3(10,11-Dihydro-dibenzo[b,f]azepin-5-yl)-N-propyl]-[2(3,4-dimethoxyphen-yl)-ethyl]methylamine; [3(10,11-Dihydro-dibenzo[b,f]azepin-5-yl)-N-propyl]-[2(4-methoxyphenyl)-ethyl]methylamine; [2(3,4-Dimethoxyphenyl)-ethyl]-[3(2-chlorphenothiazin-10-yl)-N-propyl]-m-ethylamine; [2(4-Methoxyphenyl)-ethyl]-[3(2-chlorphenothiazin-10-yl)-N-propyl]-methyl-lamine; [3(Carbazol-9-yl)-N-propyl]-[2(3,4-dimethoxyphenyl)-ethyl]methylamine; [3(Carbazol-9-yl)-N-propyl]-[2(4-methoxyphenyl)-ethyl]methylamine; [2(3,4-Dimethoxyphenyl)-ethyl]-[2(phenothiazin-10-yl)-N-ethyl]-methylamine; [2(4-Methoxyphenyl)-ethyl]-[2(phenothiazin-10-yl)—N-ethyl]-methylamine; [(3,4-Dimethoxyphenyl)-acetyl]-[3(2-chlorphenothiazin-10-yl)-N-propyl]-m-ethylamine; n(1-naphthyl)-N′ [2(3,4-dimethoxyphenyl)-ethyl]-ethyl diamine; n(1-naphthyl)-N[2(4-methoxyphenyl)-ethyl]-ethyl diamine; n[2(3,4-Dimethoxyphenyl)-ethyl]-n[1 naphthylmethyl]amine; n[2(4-Methoxyphenyl)-ethyl]-n[1-naphthylmethyl]amine; [3(10.11-Dihydro dibenzo[b,f]azepin-5-yl)-N-propyl]-[(4-methoxyphenyl)-acetyl]-methylamine;
[2(10,11-Dihydro-dibenzo[b,f]azepin-5-yl)-N-ethyl]-[2(3,4-dimethoxyphenyl)-ethyl]methylamine; [2(10,11-Dihydro-dibenzo[b,f]azepin-5-yl)-N-ethyl]-[2(4-methoxyphenyl)-ethyl]-methylamine; [2(10,11-Dihydro-dibenzo[b,f]azepin-5-yl)-N-ethyl]-[(4-methoxyphenyl)-a-cety-1]-methylamine; n[2(Carbazol-9-yl)-N-ethyl]-N′ [2(4-methoxyphenyl)-ethyl]piperazine; 1[2(Carbazol-9-yl)-N-ethyl]-4[2(4-methoxyphenyl)-ethyl]-3,5-dimethylpiperazine; [2(4-Methoxyphenyl)-ethyl]-[3(phenoxazin-10-yl)-N-propyl]-methylamine; [3(5,6,11,12-Tetrahydrodibenzo[b,f]azocin)-N-propyl]-[3(4-methoxyphenyl-)-propyl]methylamine; n(5H-Dibenzo[A,D]cycloheptan-5-yl)-N′ [2 (4-methoxyphenyl)-ethyl]-propylene diamine; and [2(Carbazol-9-yl)-N-ethyl]-[2(4-methoxyphenyl)-ethyl]methylamine

Other compounds or agents shown in the art to reduce ceramide levels include L-carnitine (200 μg/ml), siylmarin, 1-phenyl-2-decanoylaminon-3-morpholine-1-propanol, 1-phenyl-2-hexdecanoylaminon-3-pyrrolidino-1-propanol, Scyphostatin, L-camitine, glutathione, human milk bile salt-stimulated lipase, myriocin, cycloserine, Fumonisin B, PPMP, D609, methylthiodihydroceramide, propanolol, and resveratrol. Agents comprised of polypeptide sequences have also been shown to reduce ceramide levels, as describe in U.S. Pat. No. 7,037,700, incorporated herein by reference.

The foregoing listing of agents that reduce levels of ceramide or its metabolite species like LacCer is non-exhaustive. It will be apparent to one of skill in the art that analogs or fragments of the inhibitors described herein may also possess inhibitory properties. In addition to the agents described herein, the present invention may also be practiced using agents that decrease ceramide pathway metabolic enzymes or increase ceramide catabolic enzymes. These include, but are not limited to, agents that modify or regulate transcriptional or translational activity, or that otherwise degrade, inactivate, or protect these enzymes.

### IV. OTHER CERAMIDE INHIBITORS

Other embodiments of the present invention are directed to the use of antisense nucleic acids (either DNA or RNA), small interfering RNA, or microRNA to inhibit expression of the various enzymes associated with ceramide synthesis including, but not limited to, serine palmitoyltransferase, 3-ketosphinganine reductase, dihydroceramide synthase, dihydroceramide desaturase, sphingomyelinase, ceramide synthase. The term “ceramide synthesis enzymes” or “enzymes involved in the synthesis of ceramide” is used to refer collectively to any or all of the enzymes active in the synthesis of ceramide, for example, through the de novo, sphingomyelin, or salvage pathways. The term “ceramide synthesis enzyme” also refers to enzymes involved in the synthesis of ceramide metabolites or species including, but not limited to, lactosylceramide synthase (catalyzes the formation of the ceramide species, lactosylceramide (LacCer). The nucleic acid sequences encoding these enzymes are publicly available, and are incorporated herein by reference. The antisense nucleic acid can be antisense RNA, antisense DNA or small interfering RNA. Based on these known sequences, microRNA, antisense DNA or RNA that hybridize sufficiently to the respective gene or mRNA encoding the various enzymes to turn off expression can be readily designed and engineered using methods known in the art.

Accordingly, in certain aspects of the present invention, the expression of ceramide synthesis enzymes may be inhibited by the use of RNA interference techniques (RNAi). RNAi is a remarkably efficient process whereby double-stranded RNA (dsRNA) induces the sequence-specific degradation of homologous mRNA in animals and plant cells. See Hutvagner and Zamore, 12 CURR. OPIN. GENET. DEV. 225-32 (2002); Hammond et al., 2 NATURE REV. GEN. 110-19 (2001); Sharp, 15 GENES DEV. 485-90 (2001). RNAi can be triggered, for example, by nucleotide (nt) duplexes of small interfering RNA (siRNA) (Chiu et al., 10 MOL. CELL. 549-61 (2002); Elbashir et al., 411 Nature 494-98 (2001)), micro-RNAs (miRNA), functional small-hairpin RNA (shRNA), or other dsRNAs which are expressed in-vivo using DNA templates with RNA polymerase III promoters. See, e.g., Zeng et al., 9 MOL. CELL. 1327-33 (2002); Paddison et al., 16 GENES DEV. 948-58 (2002); Lee et al., 20 NATURE BIOTECHNOL. 500-05 (2002); Paul et al., 20 NATURE BIOTECHNOL. 505-08 (2002); Tuschl, 20 NATURE BIOTECHNOL. 440-48 (2002); Yu et al., 99(9) PROC. NATL. ACAD. SCI. USA, 6047-52 (2002); McManus et al., 8 RNA 842-50 (2002); Sui et al., 99(6) PROC. NATL. ACAD. SCI. USA 5515-20 (2002).

In particular embodiments, the present invention features “small interfering RNA molecules” (“siRNA molecules” or “siRNA”) and “microRNAs” (miRNAs), methods of making siRNA and miRNA molecules and methods for using siRNA and miRNA molecules (e.g., research and/or therapeutic methods). The siRNAs and miRNAs of the present invention encompass any siRNAs and miRNAs that can modulate the selective degradation of ceramide synthesis enzymes. General methods for making such molecules are described herein and are known to those of ordinary skill in the art.

In a specific embodiment, the siRNA of the present invention may comprise double-stranded small interfering RNA molecules (ds-siRNA). A ds-siRNA molecule of the present invention may be a duplex made up of a sense strand and a complementary antisense strand, the antisense strand being sufficiently complementary to a target ceramide synthesis enzyme mRNA to mediate RNAi. The siRNA molecule may comprise about 10 to about 50 or more nucleotides. More specifically, the siRNA molecule may comprise about 16 to about 30, e.g., 16, 17, 18, 19, 20, 21, 22, 23, 24, 25, 26, 27, 28, 29, or 30 nucleotides in each strand. The strands may be aligned such that there are at least 1, 2, or 3 bases at the end of the strands which do not align (e.g., for which no complementary bases occur in the opposing strand) such that an overhang of 1, 2 or 3 residues occurs at one or both ends of the duplex when strands are annealed.

In an alternative embodiment, the siRNA of the present invention may comprise single-stranded small interfering RNA molecules (ss-siRNA). Similar to the ds-siRNA molecules, the ss-siRNA molecule may comprise about 10 to about 50 or more nucleotides. More specifically, the ss-siRNA molecule may comprise about 15 to about 45 or more nucleotides. Alternatively, the ss-siRNA molecule may comprise about 19 to about 40 nucleotides. The ss-siRNA molecules of the present invention comprise a sequence that is “sufficiently complementary” to a target mRNA sequence to direct target-specific RNA interference (RNAi), as defined herein, e.g., the ss-siRNA has a sequence sufficient to trigger the destruction of the target mRNA by the RNAi machinery or process. In one embodiment, the ss-siRNA molecule can be designed such that every residue is complementary to a residue in the target molecule. Alternatively, substitutions can be made within the molecule to increase stability and/or enhance processing activity of the molecule. Substitutions can be made within the strand or can be made to residues at the ends of the strand. In a specific embodiment, the 5′-terminus may be phosphorylated (e.g., comprises a phosphate, diphosphate, or triphosphate group). In another embodiment, the 3′ end of an siRNA may be a hydroxyl group in order to facilitate RNAi, as there is no requirement for a 3′ hydroxyl group when the active agent is a ss-siRNA molecule. In other instances, the 3′ end (e.g., C3 of the 3′ sugar) of ss-siRNA molecule may lack a hydroxyl group (e.g., ss-siRNA molecules lacking a 3′ hydroxyl or C3 hydroxyl on the 3′ sugar (e.g., ribose or deoxyribose).

In another aspect, the siRNA molecules of the present invention may be modified to improve stability under in vitro and/or in vivo conditions, including, for example, in serum and in growth medium for cell cultures. In order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect the efficiency of RNA interference. For example, the absence of a 2′ hydroxyl may significantly enhance the nuclease resistance of the siRNAs in tissue culture medium.

Furthermore, the siRNAs of the present invention may include modifications to the sugar-phosphate backbone or nucleosides. These modifications can be tailored to promote selective genetic inhibition, while avoiding a general panic response reported to be generated by siRNA in some cells. In addition, modifications can be introduced in the bases to protect siRNAs from the action of one or more endogenous enzymes.

In an embodiment of the present invention, the siRNA molecule may contain at least one modified nucleotide analogue. The nucleotide analogues may be located at positions where the target-specific activity, e.g., the RNAi mediating activity is not substantially effected, e.g., in a region at the 5′-end and/or the 3′-end of the RNA molecule. Particularly, the ends may be stabilized by incorporating modified nucleotide analogues. Examples of nucleotide analogues include sugar- and/or backbone-modified ribonucleotides (e.g., include modifications to the phosphate-sugar backbone). For example, the phosphodiester linkages of natural RNA may be modified to include at least one of a nitrogen or sulfur heteroatom. In backbone-modified ribonucleotides, the phosphoester group connecting to adjacent ribonucleotides may be replaced by a modified group, e.g., a phosphothioate group. In sugar-modified ribonucleotides, the 2′ OH-group may be replaced by a group selected from H, OR, R, halo, SH, SR, NH₂, NHR, N₂ or ON, wherein R is C₁-C₆ alkyl, alkenyl or alkynyl and halo is F, Cl, Br or I.

Nucleobase-modified ribonucleotides may also be utilized, e.g., ribonucleotides containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

Derivatives of siRNAs may also be utilized herein. For example, cross-linking can be employed to alter the pharmacokinetics of the composition, e.g., to increase half-life in the body. Thus, the present invention includes siRNA derivatives that include siRNA having two complementary strands of nucleic acid, such that the two strands are crosslinked. The present invention also includes siRNA derivatives having a non-nucleic acid moiety conjugated to its 3′ terminus (e.g., a peptide), organic compositions (e.g., a dye), or the like. Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The siRNAs of the present invention can be enzymatically produced or totally or partially synthesized. Moreover, the siRNAs can be synthesized in vivo or in vitro. For siRNAs that are biologically synthesized, an endogenous or a cloned exogenous RNA polymerase may be used for transcription in vivo, and a cloned RNA polymerase can be used in vitro. siRNAs that are chemically or enzymatically synthesized are preferably purified prior to the introduction into the cell.

Although one hundred percent (100%) sequence identity between the siRNA and the target region is preferred in particular embodiments, it is not required to practice the invention. siRNA molecules that contain some degree of modification in the sequence can also be adequately used for the purpose of this invention. Such modifications may include, but are not limited to, mutations, deletions or insertions, whether spontaneously occurring or intentionally introduced.

Moreover, not all positions of a siRNA contribute equally to target recognition. In certain embodiments, for example, mismatches in the center of the siRNA may be critical and could essentially abolish target RNA cleavage. In other embodiments, the 3′ nucleotides of the siRNA do not contribute significantly to specificity of the target recognition. In particular, residues 3′ of the siRNA sequence which is complementary to the target RNA (e.g., the guide sequence) may not critical for target RNA cleavage.

Sequence identity may be determined by sequence comparison and alignment algorithms known to those of ordinary skill in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (e.g., % homology=# of identical positions/total # of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (e.g., a local alignment). A non-limiting example of a local alignment algorithm utilized for the comparison of sequences is the algorithm of Karlin and Altschul, 87 PROC. NATL. ACAD. SCI. USA 2264-68 (1990), and as modified as in Karlin and Altschul 90 PROC. NATL. ACAD. SCI. USA 5873-77 (1993). Such an algorithm is incorporated into the BLAST programs (version 2.0) of Altschul, et al., 215 J. MOL. BIOL. 403-10 (1990).

In another embodiment, the alignment may optimized by introducing appropriate gaps and determining percent identity over the length of the aligned sequences (e.g., a gapped alignment). To obtain gapped alignments for comparison purposes, Gapped BLAST can be utilized as described in Altschul et al., 25(17) NUCLEIC ACIDS RES. 3389-3402 (1997). In another embodiment, the alignment may be optimized by introducing appropriate gaps and determining percent identity over the entire length of the sequences aligned (e.g., a global alignment). A non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

In particular embodiments, greater than 90% sequence identity, e.g., 91%, 92%, 93%, 94%, 95%, 96%, 97%, 98%, 99% or even 100% sequence identity, between the siRNA and the portion of the target gene may be used. Alternatively, the siRNA may be defined functionally as a nucleotide sequence (or oligonucleotide sequence) that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing). Additional hybridization conditions include, but are not limited to, hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. The hybridization temperature for hybrids anticipated to be less than 50 base pairs in length can be about 5-10° C. less than the melting temperature (Tm) of the hybrid, where Tm is determined according to the following equations. For hybrids less than 18 base pairs in length, Tm(° C.)=2(# of A+T bases)+4(# of G+C bases). For hybrids between 18 and 49 base pairs in length, Tm(° C.)=81.5+16.6(log 10[Na⁺])+0.41(% G+C)−(600/N), where N is the number of bases in the hybrid, and [Na⁺] is the concentration of sodium ions in the hybridization buffer ([Na⁺] for 1×SSC=0.165 M). Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference. The length of the identical nucleotide sequences may be at least about 10, 12, 15, 17, 20, 22, 25, 27, 30, 32, 35, 37, 40, 42, 45, 47 50 or more bases.

Antisense molecules can act in various stages of transcription, splicing and translation to block the expression of a target gene. Without being limited by theory, antisense molecules can inhibit the expression of a target gene by inhibiting transcription initiation by forming a triple strand, inhibiting transcription initiation by forming a hybrid at an RNA polymerase binding site, impeding transcription by hybridizing with an RNA molecule being synthesized, repressing splicing by hybridizing at the junction of an exon and an intron or at the spliceosome formation site, blocking the translocation of an mRNA from nucleus to cytoplasm by hybridization, repressing translation by hybridizing at the translation initiation factor binding site or ribosome biding site, inhibiting peptide chain elongation by hybridizing with the coding region or polysome binding site of an mRNA, or repressing gene expression by hybridizing at the sites of interaction between nucleic acids and proteins. An example of an antisense oligonucleotide of the present invention is a cDNA that, when introduced into a cell, transcribes into an RNA molecule having a sequence complementary to at least part of the ceramide synthesis enzyme mRNA.

Furthermore, antisense oligonucleotides of the present invention include oligonucleotides having modified sugar-phosphodiester backbones or other sugar linkages, which can provide stability against endonuclease attacks. The present invention also encompasses antisense oligonucleotides that are covalently attached to an organic or other moiety that increase their affinity for a target nucleic acid sequence. For example, intercalating agents, alkylating agents, and metal complexes can be also attached to the antisense oligonucleotides of the present invention to modify their binding specificities.

The present invention also provides ribozymes as a tool to inhibit ceramide synthesis enzyme expression. Ribozymes are enzymatic RNA molecules capable of catalyzing the specific cleavage of RNA. The characteristics of ribozymes are well-known in the art. See, e.g., Rossi, 4 CURRENT BIOLOGY 469-71 (1994). Without being limited by theory, the mechanism of ribozyme action involves sequence specific hybridization of the ribozyme molecule to complementary target RNA, followed by an endonucleolytic cleavage. In particular embodiments, the ribozyme molecules include one or more sequences complementary to the target gene mRNA, and include the well known catalytic sequence responsible for mRNA cleavage. See U.S. Pat. No. 5,093,246. Using the known sequence of the target ceramide synthesis enzyme mRNA, a restriction enzyme-like ribozyme can be prepared using standard techniques.

The expression of a ceramide synthesis enzyme gene can also be inhibited by using triple helix formation. Nucleic acid molecules to be used in triple helix formation for the inhibition of transcription can be single stranded and composed of deoxynucleotides. The base composition of these oligonucleotides must be designed to promote triple helix formation via Hoogsteen base paring rules, which generally require sizeable stretches of either purines or pyrimidines to be present on one strand of a duplex. Nucleotide sequences may be pyrimidine-based, which will result in TAT and CGC⁺ triplets across the three associated strands of the resulting triple helix. The pyrimidine-rich molecules provide base complementarity to a purine-rich region of a single strand of the duplex in a parallel orientation to that strand. In addition, nucleic acid molecules that are purine-rich, e.g., containing a stretch of G residues, may be chosen. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so-called “switchback” nucleic acid molecule. Switchback molecules are synthesized in an alternating 5′-3′,3′-5′ manner, such that they base pair first with one strand of a duplex and then the other, eliminating the necessity for a sizeable stretch of either purines or pyrimidines to be present on one strand of a duplex.

The expression of ceramide synthesis enzymes may be also inhibited by what is referred to as “co-repression.” Co-repression refers to the phenomenon in which, when a gene having an identical or similar to the target sequence is introduced to a cell, expression of both introduced and endogenous genes becomes repressed. This phenomenon, although first observed in plant system, has been observed in certain animal systems as well. The sequence of the gene to be introduced does not have to be identical to the target sequence, but sufficient homology allows the co-repression to occur. The determination of the extent of homology depends on individual cases, and is within the ordinary skill in the art.

It would be readily apparent to one of ordinary skill in the art that other methods of gene expression inhibition that selectively target a ceramide synthesis enzyme DNA or mRNA can also be used in connection with this invention without departing from the spirit of the invention. In a specific embodiment, using techniques known to those of ordinary skill in the art, the present invention contemplates affecting the promoter region of a ceramide synthesis enzyme gene to effectively switch off transcription.

One or more of the following guidelines may be used in designing the sequence of siRNA and other nucleic acids designed to bind to a target ceramide synthesis enzyme mRNA, e.g., shRNA, stRNA, antisense oligonucleotides, ribozymes, and the like, that are advantageously used in accordance with the present invention.

Beginning with the AUG start codon of a ceramide synthesis enzyme gene, each AA dinucleotide sequence and the 3′ adjacent 16 or more nucleotides are potential siRNA targets. In a specific embodiment, the siRNA is specific for a target region that differs by at least one base pair between the wild type and mutant allele or between splice variants. In dsRNAi, the first strand is complementary to this sequence, and the other strand identical or substantially identical to the first strand. siRNAs with lower G/C content (35-55%) may be more active than those with G/C content higher than 55%. Thus in one embodiment, the invention includes nucleic acid molecules having 35-55% G/C content. In addition, the strands of the siRNA can be paired in such a way as to have a 3′ overhang of 1 to 4, e.g., 2, nucleotides. Thus in another embodiment, the nucleic acid molecules may have a 3′ overhang of 2 nucleotides, such as TT. The overhanging nucleotides may be either RNA or DNA. In one embodiment, it may be desirable to choose a target region wherein the mismatch is a purine:purine mismatch.

Using any method known in the art, compare the potential targets to the appropriate genome database (human, mouse, rat, etc.) and eliminate from consideration any target sequences with significant homology to other coding sequences. One such method for such sequence homology searches is known as BLAST, which is available at National Center for Biotechnology Information website (http://www.ncbi.nih.gov). Select one or more sequences that meet the criteria for evaluation.

Another method includes selecting in the sequence of the target mRNA, a region located from about 50 to about 100 nt 3′ from the start codon. In this region, search for the following sequences: AA(N19)TT or AA(N21), where N=any nucleotide. The GC content of the selected sequence should be from about 30% to about 70%, preferably about 50%. To maximize the specificity of the RNAi, it may be desirable to use the selected sequence in a search for related sequences in the genome of interest; sequences absent from other genes are preferred. The secondary structure of the target mRNA may be determined or predicted, and it may be preferable to select a region of the mRNA that has little or no secondary structure, but it should be noted that secondary structure seems to have little impact on RNAi. When possible, sequences that bind transcription and/or translation factors should be avoided, as they might competitively inhibit the binding of a siRNA, sbRNA or stRNA (as well as other antisense oligonucleotides) to the mRNA. Further general information about the design and use of siRNA may be found in “The siRNA User Guide,” available at The Max-Planck-Institut fur Biophysikalishe Chemie website (http://www.mpibpc.mpg.de).

Negative control siRNAs should have the same nucleotide composition as the selected siRNA, but without significant sequence complementarity to the appropriate genome. Such negative controls may be designed by randomly scrambling the nucleotide sequence of the selected siRNA; a homology search can be performed to ensure that the negative control lacks homology to any other gene in the appropriate genome.

Delivery of the compositions of the present invention (e.g., siRNAs, antisense oligonucleotides, or other compositions described herein) into a patient can either be direct, e.g., the patient is directly exposed to the compositions of the present invention or compound-carrying vector, or indirect, e.g., cells are first transformed with the compositions of this invention in vitro, then transplanted into the patient for cell replacement therapy. These two approaches are known as in vivo and ex vivo therapy, respectively.

In the case of in vivo therapy, the compositions of the present invention are directly administered in vivo, where they are expressed to produce the encoded product. This can be accomplished by any of numerous methods known in the art, e.g., by constructing them as part of an appropriate nucleic acid expression vector and administering them so that they become intracellular, by infection using a defective or attenuated retroviral or other viral vector, by direct injection of naked DNA, by coating with lipids or cell-surface receptors or transfecting agents, encapsulation in liposomes, nanoparticles, microparticles, or microcapsules, by administering them in linkage to a peptide which is known to enter the cell or nucleus, or by administering them in linkage to a ligand subject to receptor-mediated endocytosis which can be used to target cell types specifically expressing the receptors. Further, the compositions of the present invention can be targeted in vivo for cell specific uptake and expression, by targeting a specific receptor. See, e.g., WO93/14188, WO 93/20221, WO 92/22635, WO92/20316, and WO 92/06180.

Ex vivo therapy involves transferring the compositions of the present invention to cells in tissue culture by methods well-known in the art such as electroporation, transfection, lipofection, microinjection, calcium phosphate mediated transfection, nanosystems, cell fusion, chromosome-mediated gene transfer, microcell-mediated gene transfer, spheroplast fusion, and infection with a viral vector containing the nucleic acid sequences. These techniques should provide for the stable transfer of the compositions of this invention to the cell, so that they are expressible by the cell and preferably heritable and expressible by its cell progeny. In particular embodiments, the method of transfer includes the transfer of a selectable marker to the cells. The cells are then placed under selection to isolate those cells that have taken up and are expressing the transferred compositions. The resulting recombinant cells can be delivered to a patient by various methods known in the art. Examples of the delivery methods include, but are not limited to, subcutaneous injection, skin graft, and intravenous injection.

### V. DETECTION AND MEASUREMENT OF CFTR LEVELS AND/OR FUNCTIONAL ACTIVITY

In particular embodiments, CFTR and optionally other biomarkers (at times, collectively referred to as “biomarkers”) can be measured by immunoassay. Immunoassay requires biospecific capture reagents, such as antibodies, to capture the biomarkers. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well known in the art.

The present invention contemplates traditional immunoassays including, for example, sandwich immunoassays including ELISA or fluorescence-based immunoassays, as well as other assays to measure membrane/lipid-raft-CFTR levels and activity. Nephelometry is an assay performed in liquid phase, in which antibodies are in solution. Binding of the antigen to the antibody results in changes in absorbance, which is measured. In the SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry. The Quantikine immunoassay developed by R&D Systems, Inc. (Minneapolis, Minn.) may also be used in the methods of the present invention.

In several embodiments, CFTR and optionally other biomarkers may be detected by means of an electrochemicaluminescent assay developed by Meso Scale Discovery (Gaithersburg, Md.) or similar functional activity assays. Electrochemiluminescence detection uses labels that emit light when electrochemically stimulated. Background signals are minimal because the stimulation mechanism (electricity) is decoupled from the signal (light). Labels are stable, non-radioactive and offer a choice of convenient coupling chemistries. They emit light at ˜620 nm, eliminating problems with color quenching. See U.S. Pat. No. 7,497,997; No. 7,491,540; No. 7,288,410; No. 7,036,946; No. 7,052,861; No. 6,977,722; No. 6,919,173; No. 6,673,533; No. 6,413,783; No. 6,362,011; No. 6,319,670; No. 6,207,369; No. 6,140,045; No. 6,090,545; and No. 5,866,434. See also U.S. Patent Applications Publication No. 2009/0170121; No. 2009/006339; No. 2009/0065357; No. 2006/0172340; No. 2006/0019319; No. 2005/0142033; No. 2005/0052646; No. 2004/0022677; No. 2003/0124572; No. 2003/0113713; No. 2003/0003460; No. 2002/0137234; No. 2002/0086335; and No. 2001/0021534.

CFTR and optionally other biomarkers can be detected by other suitable methods. Detection paradigms that can be employed to this end include optical methods, electrochemical methods (voltametry and amperometry techniques), atomic force microscopy, spectroscopy (time-of-flight, magnetic sector, quadrupole filter, ion trap, ion cyclotron resonance, electrostatic sector analyzer and hybrids of the foregoing, surface enhanced laser desorption and ionization (SELDI), Affinity Capture Mass Spectrometry (also called Surface-Enhanced Affinity Capture (SEAC)), Surface-Enhanced Neat Desorption (SEND), Surface-Enhanced Photolabile Attachment and Release (SEPAR), and Matrix-Assisted Laser Desorption/Ionization (MALDI), and radio frequency methods (e.g., multipolar resonance spectroscopy). Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

Furthermore, a sample may also be analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there. Protein biochips are biochips adapted for the capture of polypeptides. Many protein biochips are described in the art. These include, for example, protein biochips produced by Ciphergen Biosystems, Inc. (Fremont, Calif.), Zyomyx (Hayward, Calif.), Invitrogen (Carlsbad, Calif.), Biacore (Uppsala, Sweden) and Procognia (Berkshire, UK). Examples of such protein biochips are described in the following patents or published patent applications: U.S. Pat. No. 6,537,749; U.S. Pat. No. 6,329,209; U.S. Pat. No. 6,225,047; U.S. Pat. No. 5,242,828; PCT International Publication No. WO 00/56934; and PCT International Publication No. WO 03/048768.

### VI. KITS FOR THE DETECTION OF CFTR AND OTHER BIOMARKERS

In another aspect, the present invention provides kits used to detect CFTR and optionally other biomarkers. In a specific embodiment, the kit is provided as an ELISA kit comprising an antibody to CFTR. The ELISA kit may comprise a solid support, such as a chip, microtiter plate (e.g., a 96-well plate), bead, or resin having a CFTR capture reagent attached thereon. The kit may further comprise a means for detecting CFTR, such as an anti-CFTR antibody, and a secondary antibody-signal complex such as horseradish peroxidase (HRP)-conjugated goat anti-rabbit IgG antibody and tetramethyl benzidine (TMB) as a substrate for HRP.

The kit may be provided as an immuno-chromatography strip comprising a membrane on which CFTR antibody is immobilized, and a means for detecting CFTR, e.g., a gold particle bound CFTR antibody, where the membrane, includes NC membrane and PVDF membrane. The kit may comprise a plastic plate on which a sample application pad, a gold particle bound CFTR antibody temporally immobilized on a glass fiber filter, a nitrocellulose membrane on which a CFTR antibody band and a secondary antibody band are immobilized and an absorbent pad are positioned in a serial manner, so as to keep continuous capillary flow of blood serum.

The kit can also comprise a washing solution or instructions for making a washing solution, in which the combination of the capture reagent and the washing solution allows capture of the biomarker or biomarkers on the solid support for subsequent detection by, e.g., an antibody or mass spectrometry. In a further embodiment, a kit can comprise instructions for suitable operational parameters in the form of a label or separate insert. For example, the instructions may inform a consumer about how to collect the sample, how to wash the probe or the particular biomarkers to be detected. In yet another embodiment, the kit can comprise one or more containers with biomarker samples, to be used as standard(s) for calibration.

### VII. PHARMACEUTICAL COMPOSITIONS AND ADMINISTRATION

Accordingly, a pharmaceutical composition of the present invention may comprise an effective amount of at least one ceramide inhibitor. As used herein, the term “effective” means adequate to accomplish a desired, expected, or intended result. More particularly, the terms “effective amount” and “therapeutically effective amount” are used interchangeably and refer to an amount of at least one ceramide inhibitor, perhaps in further combination with a second ceramide inhibitor and/or optionally another therapeutic agent, necessary to provide the desired treatment or therapeutic effect, e.g., an amount that is effective to prevent, alleviate, treat or ameliorate symptoms of a disease or prolong the survival of the subject being treated. In particular embodiments, the pharmaceutical compositions of the present invention are administered in a therapeutically effective amount to treat a subject suffering from a pulmonary condition. As would be appreciated by one of ordinary skill in the art, the exact amount required will vary from subject to subject, depending on age, general condition of the subject, the severity of the condition being treated, the particular compound and/or composition administered, and the like. An appropriate “therapeutically effective amount” in any individual case can be determined by one of ordinary skill in the art by reference to the pertinent texts and literature and/or by using routine experimentation.

The pharmaceutical compositions of the present invention are in biologically compatible forms suitable for administration in vivo to subjects. The pharmaceutical compositions can further comprise a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the at least one ceramide inhibitor is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water may be a carrier when the pharmaceutical composition is administered orally. Saline and aqueous dextrose may be carriers when the pharmaceutical composition is administered intravenously. Saline solutions and aqueous dextrose and glycerol solutions may be employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried slim milk, glycerol, propylene, glycol, water, ethanol and the like. The pharmaceutical composition may also contain minor amounts of wetting or emulsifying agents, or pH buffering agents.

The pharmaceutical compositions of the present invention can take the form of solutions, suspensions, emulsions, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. In a specific embodiment, a pharmaceutical composition comprises an effective amount of at least one ceramide inhibitor together with a suitable amount of a pharmaceutically acceptable carrier so as to provide the form for proper administration to the patient. In particular embodiments that comprise the administration of two or more ceramide inhibitors (or a ceramide inhibitor and another therapeutic agent), the ceramide inhibitors (or a ceramide inhibitor and another therapeutic agent) can be separately formulated and administered according to the present invention. The formulation should suit the mode of administration.

In general, the pharmaceutical compositions comprising at least one membrane/lipid raft-CFTR agonist and/or at least one ceramide inhibitor disclosed herein may be used alone (i.e., two co-administered ceramide inhibitors) or in concert with other therapeutic agents at appropriate dosages defined by routine testing in order to obtain optimal efficacy while minimizing any potential toxicity. The dosage regimen utilizing a pharmaceutical composition of the present invention may be selected in accordance with a variety of factors including type, species, age, weight, sex, medical condition of the patient; the severity of the condition to be treated; the route of administration; the renal and hepatic function of the patient; and the particular pharmaceutical composition employed. A physician of ordinary skill can readily determine and prescribe the effective amount of the pharmaceutical composition (and potentially other agents including therapeutic agents) required to prevent, counter, or arrest the progress of the condition.

Optimal precision in achieving concentrations of the therapeutic regimen (e.g., pharmaceutical compositions comprising at least one membrane/lipid raft-CFTR agonist and/or at least one ceramide inhibitor in combination with another therapeutic agent) within the range that yields maximum efficacy with minimal toxicity may require a regimen based on the kinetics of the pharmaceutical composition's availability to one or more target sites. Distribution, equilibrium, and elimination of a pharmaceutical composition may be considered when determining the optimal concentration for a treatment regimen. The dosages of a pharmaceutical composition disclosed herein may be adjusted when combined to achieve desired effects. On the other hand, dosages of the pharmaceutical compositions and various therapeutic agents may be independently optimized and combined to achieve a synergistic result wherein the pathology is reduced more than it would be if either was used alone.

In particular, toxicity and therapeutic efficacy of a pharmaceutical composition disclosed herein may be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effect is the therapeutic index and it may be expressed as the ratio LD₅₀/ED₅₀. Pharmaceutical compositions exhibiting large therapeutic indices are preferred except when cytotoxicity of the composition is the activity or therapeutic outcome that is desired. Although pharmaceutical compositions that exhibit toxic side effects may be used, a delivery system can target such compositions to the site of affected tissue in order to minimize potential damage to uninfected cells and, thereby, reduce side effects. Generally, the pharmaceutical compositions of the present invention may be administered in a manner that maximizes efficacy and minimizes toxicity.

Data obtained from cell culture assays and animal studies may be used in formulating a range of dosages for use in humans. The dosages of such compositions lie preferably within a range of circulating concentrations that include the ED₅₀ with little or no toxicity. The dosage may vary within this range depending upon the dosage form employed and the route of administration utilized. For any composition used in the methods of the present invention, the therapeutically effective dose may be estimated initially from cell culture assays. A dose may be formulated in animal models to achieve a circulating plasma concentration range that includes the IC₅₀ (the concentration of the test composition that achieves a half-maximal inhibition of symptoms) as determined in cell culture. Such information may be used to accurately determine useful doses in humans. Levels in plasma may be measured, for example, by high performance liquid chromatography.

Moreover, the dosage administration of the compositions of the present invention may be optimized using a pharmacokinetic/pharmacodynamic modeling system. For example, one or more dosage regimens may be chosen and a pharmacokinetic/pharmacodynamic model may be used to determine the pharmacokinetic/pharmacodynamic profile of one or more dosage regimens. Next, one of the dosage regimens for administration may be selected which achieves the desired pharmacokinetic/pharmacodynamic response based on the particular pharmacokinetic/pharmacodynamic profile. See WO 00/67776, which is entirely expressly incorporated herein by reference.

More specifically, the pharmaceutical compositions may be administered in a single daily dose, or the total daily dosage may be administered in divided doses of two, three, or four times daily. In the case of oral administration, the daily dosage of the compositions may be varied over a wide range from about 0.1 ng to about 1,000 mg per patient, per day. The range may more particularly be from about 0.001 ng/kg to 10 mg/kg of body weight per day, about 0.1-100 μg, about 1.0-50 μg or about 1.0-20 mg per day for adults (at about 60 kg).

The daily dosage of the pharmaceutical compositions may be varied over a wide range from about 0.1 ng to about 1000 mg per adult human per day. For oral administration, the compositions may be provided in the form of tablets containing from about 0.1 ng to about 1000 mg of the composition or 0.1, 0.2, 0.5, 1.0, 2.0, 5.0, 10.0, 15.0, 100, 150, 200, 250, 300, 350, 400, 450, 500, 550, 600, 650, 700, 800, 900, or 1000 milligrams of the composition for the symptomatic adjustment of the dosage to the patient to be treated. An effective amount of the pharmaceutical composition is ordinarily supplied at a dosage level of from about 0.1 ng/kg to about 20 mg/kg of body weight per day. In one embodiment, the range is from about 0.2 ng/kg to about 10 mg/kg of body weight per day. In another embodiment, the range is from about 0.5 ng/kg to about 10 mg/kg of body weight per day. The pharmaceutical compositions may be administered on a regimen of about 1 to about 10 times per day.

In the case of injections, it is usually convenient to give by an intravenous route in an amount of about 0.0001 μg-30 mg, about 0.01 μg-20 mg or about 0.01-10 mg per day to adults (at about 60 kg). In the case of other animals, the dose calculated for 60 kg may be administered as well.

Doses of a pharmaceutical composition of the present invention can optionally include 0.0001 μg to 1,000 mg/kg/administration, or 0.001 μg to 100.0 mg/kg/administration, from 0.01 μg to 10 mg/kg/administration, from 0.1 μg to 10 mg/kg/administration, including, but not limited to, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100-500 mg/kg/administration or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 μg/ml serum concentration per single or multiple administration or any range, value or fraction thereof.

As a non-limiting example, treatment of subjects can be provided as a one-time or periodic dosage of a composition of the present invention 0.1 ng to 100 mg/kg such as 0.0001, 0.001, 0.01, 0.1 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses.

Specifically, the pharmaceutical compositions of the present invention may be administered at least once a week over the course of several weeks. In one embodiment, the pharmaceutical compositions are administered at least once a week over several weeks to several months. In another embodiment, the pharmaceutical compositions are administered once a week over four to eight weeks. In yet another embodiment, the pharmaceutical compositions are administered once a week over four weeks.

More specifically, the pharmaceutical compositions may be administered at least once a day for about 2 days, at least once a day for about 3 days, at least once a day for about 4 days, at least once a day for about 5 days, at least once a day for about 6 days, at least once a day for about 7 days, at least once a day for about 8 days, at least once a day for about 9 days, at least once a day for about 10 days, at least once a day for about 11 days, at least once a day for about 12 days, at least once a day for about 13 days, at least once a day for about 14 days, at least once a day for about 15 days, at least once a day for about 16 days, at least once a day for about 17 days, at least once a day for about 18 days, at least once a day for about 19 days, at least once a day for about 20 days, at least once a day for about 21 days, at least once a day for about 22 days, at least once a day for about 23 days, at least once a day for about 24 days, at least once a day for about 25 days, at least once a day for about 26 days, at least once a day for about 27 days, at least once a day for about 28 days, at least once a day for about 29 days, at least once a day for about 30 days, or at least once a day for about 31 days.

Alternatively, the pharmaceutical compositions may be administered about once every day, about once every 2 days, about once every 3 days, about once every 4 days, about once every 5 days, about once every 6 days, about once every 7 days, about once every 8 days, about once every 9 days, about once every 10 days, about once every 11 days, about once every 12 days, about once every 13 days, about once every 14 days, about once every 15 days, about once every 16 days, about once every 17 days, about once every 18 days, about once every 19 days, about once every 20 days, about once every 21 days, about once every 22 days, about once every 23 days, about once every 24 days, about once every 25 days, about once every 26 days, about once every 27 days, about once every 28 days, about once every 29 days, about once every 30 days, or about once every 31 days.

The pharmaceutical compositions of the present invention may alternatively be administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, about once every 12 weeks, about once every 13 weeks, about once every 14 weeks, about once every 15 weeks, about once every 16 weeks, about once every 17 weeks, about once every 18 weeks, about once every 19 weeks, about once every 20 weeks.

Alternatively, the pharmaceutical compositions of the present invention may be administered about once every month, about once every 2 months, about once every 3 months, about once every 4 months, about once every 5 months, about once every 6 months, about once every 7 months, about once every 8 months, about once every 9 months, about once every 10 months, about once every 11 months, or about once every 12 months.

Alternatively, the pharmaceutical compositions may be administered at least once a week for about 2 weeks, at least once a week for about 3 weeks, at least once a week for about 4 weeks, at least once a week for about 5 weeks, at least once a week for about 6 weeks, at least once a week for about 7 weeks, at least once a week for about 8 weeks, at least once a week for about 9 weeks, at least once a week for about 10 weeks, at least once a week for about 11 weeks, at least once a week for about 12 weeks, at least once a week for about 13 weeks, at least once a week for about 14 weeks, at least once a week for about 15 weeks, at least once a week for about 16 weeks, at least once a week for about 17 weeks, at least once a week for about 18 weeks, at least once a week for about 19 weeks, or at least once a week for about 20 weeks.

Alternatively the pharmaceutical compositions may be administered at least once a week for about 1 month, at least once a week for about 2 months, at least once a week for about 3 months, at least once a week for about 4 months, at least once a week for about 5 months, at least once a week for about 6 months, at least once a week for about 7 months, at least once a week for about 8 months, at least once a week for about 9 months, at least once a week for about 10 months, at least once a week for about 11 months, or at least once a week for about 12 months.

The pharmaceutical compositions of the present invention (e.g., agents such as a membrane/lipid raft-CFTR agonist and/or a ceramide inhibitor such as Fumonisin-B1 (FB1) or Amitriptyline (AMT)) can be administered simultaneously or sequentially by the same or different routes of administration. The pharmaceutical compositions may further be combined with one or more additional therapeutic agents including, but not limited to, immunomodulatory agents, anti-inflammatory agents (e.g., adrenocorticoids, corticosteroids (e.g., beclomethasone, budesonide, flunisolide, fluticasone, triamcinolone, methlyprednisolone, prednisolone, prednisone, hydrocortisone), glucocorticoids, steroids, non-steriodal anti-inflammatory drugs (e.g., aspirin, ibuprofen, diclofenac, and COX-2 inhibitors), and leukotreine antagonists (e.g., montelukast, methyl xanthines, zafirlukast, and zileuton), beta2-agonists (e.g., albuterol, biterol, fenoterol, isoetharie, metaproterenol, pirbuterol, salbutamol, terbutalin formoterol, salmeterol, and salbutamol terbutaline), anticholinergic agents (e.g., ipratropium bromide and oxitropium bromide), sulphasalazine, penicillamine, dapsone, antihistamines, anti-malarial agents (e.g., hydroxychloroquine), anti-viral agents, and antibiotics (e.g., dactinomycin (formerly actinomycin), bleomycin, erythomycin, penicillin, mithramycin, and anthramycin (AMC)).

The determination of the identity and amount of the pharmaceutical compositions for use in the methods of the present invention can be readily made by ordinarily skilled medical practitioners using standard techniques known in the art. In specific embodiments, an effective amount of a first ceramide inhibitor of the present invention can be administered in combination with an effective amount of a second ceramide inhibitor. In other specific embodiments, a first ceramide inhibitor and a second ceramide inhibitor can be administered in combination with an effective amount of another therapeutic agent.

In various embodiments, an agent of the present invention (e.g., a membrane/lipid raft-CFTR agonist and/or a ceramide inhibitor (and optionally another membrane/lipid raft-CFTR agonist and/or ceramide inhibitor and/or another therapeutic agent)) may be administered at about the same time, less than 1 minute apart, less than 2 minutes apart, less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In particular embodiments, two or more therapies are administered within the same patient visit.

In certain embodiments, an agent of the present invention (e.g., a membrane/lipid raft-CFTR agonist and/or a ceramide inhibitor (and optionally another membrane/lipid raft-CFTR agonist and/or ceramide inhibitor and/or another therapeutic agent)) are cyclically administered. Cycling therapy involves the administration of a first therapy (e.g., first ceramide inhibitor) for a period of time, followed by the administration of a second therapy (e.g., a second ceramide inhibitor) for a period of time, optionally, followed by the administration of perhaps a third therapy (e.g., another therapeutic agent) for a period of time and so forth, and repeating this sequential administration, e.g., the cycle, in order to reduce the development of resistance to one of the therapies, to avoid or reduce the side effects of one of the therapies, and/or to improve the efficacy of the therapies. In certain embodiments, the administration of the combination therapy of the present invention may be repeated and the administrations may be separated by at least 1 day, 2 days, 3 days, 5 days, 10 days, 15 days, 30 days, 45 days, 2 months, 75 days, 3 months, or at least 6 months.

### VIII. ROUTES OF ADMINISTRATION

The pharmaceutical compositions of the present invention may be administered by any particular route of administration including, but not limited to oral, parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intraosseous, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, iontophoretic means, or transdermal means. In particular embodiments, oral administration or injection (e.g., subcutaneous) are suitable.

In other embodiments, the pharmaceutical compositions of the present invention are prepared for pulmonary/nasal administration. There are a several desirable features of an inhalation device for administering a pharmaceutical composition of the present invention. For example, delivery by the inhalation device is reliable, reproducible, and accurate. For pulmonary administration, at least one pharmaceutical composition may be delivered in a particle size effective for reaching the lower airways of the lung or sinuses. The inhalation device can optionally deliver small dry particles, e.g. less than about 10 μm, including about 1-5 μm, for good respirability.

According to the present invention, at least one pharmaceutical composition can be delivered by any of a variety of inhalation or nasal devices known in the art for administration of a therapeutic agent by inhalation. Devices capable of depositing aerosolized formulations in the sinus cavity or alveoli of a patient include metered dose inhalers, nebulizers, dry powder generators, sprayers, and the like. Other devices suitable for directing pulmonary or nasal administration are also known in the art.

All such devices can be used for the administration of a composition in an aerosol. Such aerosols may comprise either solutions (both aqueous and non aqueous) or solid particles. Metered dose inhalers like the Ventolin® metered dose inhaler, typically use a propellent gas and require actuation during inspiration. See, e.g., WO 98/35888; WO 94/16970. Dry powder inhalers like Turbuhaler® (Astra), Rotahaler® (Glaxo), Diskus® (Glaxo), Spiros® inhaler

(Dura), devices marketed by Inhale Therapeutics, and the Spinhaler® powder inhaler (Fisons), use breath-actuation of a mixed powder. See U.S. Pat. Nos. 5,458,135; 4,668,218; WO 97/25086; WO 94/08552; WO 94/06498; and EP 0 237 507, each entirely expressly incorporated herein by reference. Nebulizers like AERx®, Aradigm, the Ultravent® nebulizer (Mallinckrodt), and the Acorn II® nebulizer (Marquest Medical Products), produce aerosols from solutions, while metered dose inhalers, dry powder inhalers, etc., generate small particle aerosols. These specific examples of commercially available inhalation devices are intended to be a representative of specific devices suitable for the practice of the invention, and are not intended as limiting the scope of the invention.

Formulations suitable for nasal administration, wherein the carrier is a solid, include a coarse powder having a particle size, for example, in the range of 0.001 to 5000 μm which is administered in the manner in which snuff is administered, i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the composition.

A spray comprising a pharmaceutical composition of the present invention can be produced by forcing a suspension or solution of a composition disclosed herein through a nozzle under pressure. The nozzle size and configuration, the applied pressure, and the liquid feed rate can be chosen to achieve the desired output and particle size. An electrospray can be produced, for example, by an electric field in connection with a capillary or nozzle feed. Advantageously, particles of at least one composition delivered by a sprayer have a particle size in a range of about less than 1 nm to less than about 200 μm.

Pharmaceutical compositions of the present invention suitable for use with a sprayer typically include a composition disclosed herein in an aqueous solution at a concentration of about 0.0001 μg to about 100 mg of a composition disclosed herein per ml of solution, or any range or value therein, including, but not limited to, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 μg/ml or mg/ml. The pharmaceutical composition can include agents such as an excipient, a buffer, an isotonicity agent, a preservative, a surfactant, or other known agents of pharmaceutical compositions.

A pharmaceutical composition of the present invention can also be administered by a nebulizer such as a jet nebulizer or an ultrasonic nebulizer. Typically, in a jet nebulizer, a compressed air source is used to create a high-velocity air jet through an orifice. As the gas expands beyond the nozzle, a low-pressure region is created, which draws a solution of composition protein through a capillary tube connected to a liquid reservoir. The liquid stream from the capillary tube is sheared into unstable filaments and droplets as it exits the tube, creating the aerosol. A range of configurations, flow rates, and baffle types can be employed to achieve the desired performance characteristics from a given jet nebulizer. In an ultrasonic nebulizer, high-frequency electrical energy is used to create vibrational, mechanical energy, typically employing a piezoelectric transducer. This energy is transmitted to the formulation either directly or through a coupling fluid, creating an aerosol including the composition.

Advantageously, the pharmaceutical composition delivered by a nebulizer have a particle size range of from about less than 1 nm to less than about 2000 μm.

Pharmaceutical compositions of the present invention suitable for use with a nebulizer, either jet or ultrasonic, typically include a concentration of about 0.1 ng to about 100 mg of a pharmaceutical composition disclosed herein per ml of solution, or any range or value therein including, but not limited to, the individual amounts disclosed for spray compositions. The pharmaceutical composition can include other pharmaceutical agents such as an excipient, a buffer, an isotonicity agent, a preservative, a surfactant, and those known in the art for use in nebulizer administration.

In a metered dose inhaler (MDI), a propellant, a pharmaceutical composition of the present invention, and any excipients or other additives are contained in a canister as a mixture including a liquefied, compressed gas. Actuation of the metering valve releases the mixture as an aerosol, preferably containing a particle size range of from about less than 1 nm to less than about 2000 μm.

The desired aerosol particle size or nano-encapsulation can be obtained by employing a formulation of a pharmaceutical composition of the present invention produced by various methods known to those of skill in the art including, but not limited to, jet-milling, spray drying, critical point condensation, and the like. Suitable metered dose inhalers include those manufactured by 3M or Glaxo and employing a hydrofluorocarbon propellant. Pharmaceutical compositions for use with a metered-dose inhaler device will generally include a finely divided powder containing a composition disclosed herein as a suspension in a non-aqueous medium, for example, suspended in a propellant with the aid of a surfactant. The propellant can be any conventional material employed for this purpose such as chlorofluorocarbon, a hydrochlorofluorocarbon, a hydrofluorocarbon, or a hydrocarbon including trichlorofluoromethane, dichlorodifluoromethane, dichlorotetrafluoroethanol and 1,1,1,2-tetrafluoroethane, HFA-134a (hydrofluoroalkane-134a), HFA-227 (hydrofluoroalkane-227), or the like. In one embodiment, the propellant is a hydrofluorocarbon. The surfactant can be chosen to stabilize the composition of the present invention as a suspension in the propellant, to protect the active agent against chemical degradation, and the like. Suitable surfactants include sorbitan trioleate, soya lecithin, oleic acid, or the like. In some cases solution aerosols are preferred using solvents such as ethanol. One of ordinary skill in the art will recognize that the methods of the present invention can be achieved by pulmonary administration of a composition disclosed herein via devices not described herein.

For absorption through mucosal surfaces, the compositions and methods of the present invention for administering a pharmaceutical composition disclosed herein include an emulsion comprising a plurality of submicron particles, a mucoadhesive macromolecule, a bioactive peptide, and an aqueous continuous phase, which promotes absorption through mucosal surfaces by achieving mucoadhesion of the emulsion particles. See, e.g., U.S. Pat. No. 5,514,670. Mucous surfaces suitable for application of the emulsions of the present invention can include corneal, conjunctival, buccal, sublingual, nasal, vaginal, pulmonary, abdominal, intestinal, and rectal routes of administration. Compositions for vaginal or rectal administration such as suppositories, can contain as excipients, for example, polyalkyleneglycols, vaseline, cocoa butter, and the like. Compositions for intranasal administration can be solid and contain excipients, for example, lactose or can be aqueous or oily solutions of nasal drops. For buccal administration, excipients include sugars, calcium stearate, magnesium stearate, pregelinatined starch, and the like. See, e.g., U.S. Pat. No. 5,849,695.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Critical Modifier Role of Membrance-Cystic Fibrosis Transmembrance Conductance Regulator-Dependent Ceramide Signaling in Lung Injury and Emphysema

### Materials and Methods

Reagents and Treatments.

The cells were cultured at 37° C. with 5% CO² in MEM [(CFBE4lo-, CFBE4lo-wtCFTR (from Dr. Dieter Gruenert, Univ. of California under material transfer agreement)], DMEM/F12 (HEK-293) or RPMI-1640 [Jurkat (ATCC T1B-152), splenocytes, neutrophils and macrophages] media, supplemented with 10% Fetal Bovine Serum (FBS) and 1% Penicillin, Streptomycin and Amphotericin B (PSA) both from Invitrogen. The P. aureginosa-LPS (Pa-LPS; Sigma), Fumonisin-B1 (FB1, Cayman Chemicals), Amitriptyline (AMT, Sigma), Methyl-β-cyclodextrin (CD, Sigma), Concanavalin A (ConA, Sigma), TNFα (Invitrogen), and cigarette smoke extract (CSE, Murty Pharmaceuticals Inc.) treatments were used for the indicated time points. For in vitro experiments, cells were treated with 10 ng/ml Pa-LPS, 50 μM FB1, 50 μM AMT, 5 mM CD, 5 or 10 μg/ml ConA, 10 ng/ml TNFα and 0-160 μg/ml CSE as described. Mice were treated by intratracheal (i.t.) instillation with 20 μg Pa-LPS, 50 μg FB1, 50 μg AMT and 50 μg CD as indicated in 100 μl total volume of PBS, while control mice received PBS alone.

Mice Experiments and Human Subjects.

All animal experiments were carried out in accordance with the Johns Hopkins University (JHU) Animal Care and Use Committee (ACUC) approved protocols. Age-, weight-, and sex-matched (24 wk old), B6-12956-Cftr^{-/-} (Cftrtm^{1Kthc}-TgN(^{FABPCFTR})) (30, 31) and Cftr^{+/+} inbred mice strains (procured from Case Western Reserve University Animal Resource Center, Cleveland, Ohio; n=3-5 for all experiments). The changes in cytokine and inflammatory markers between Cftr^{+/+}and Cftr^{-/-} mice were verified by multiple (2, 3) experiments, and representative data are shown. All mice were housed in a controlled environment and pathogen-free conditions. Lung injury was induced in these mice by intra-tracheal (i.t.) instillation of Pa-LPS (20 μg in 100 ml PBS) for 12 h, which resulted in ˜1-2 g loss in body weight. The de novo ceramide synthesis or membrane-ceramide release was partially inhibited by i.t. (50 μg in 100 μA PBS) FB1 or AMT administration 12 h after Pa-LPS treatment. Mice were sacrificed 24 h after drug treatment, and the bronchoalveolar lavage fluid (BALF) was collected for cytokine ELISAs. The lungs were fixed in 10% buffered formalin phosphate (Fisher Scientific, Pittsburgh, Pa.), paraffin embedded, and cut into longitudinal sections (5 μm thick) on glass slides for immunostaining. For depletion of membrane-CFTR in murine lungs, 72-h i.t. CD treatment was used, and lung tissues were collected as above. The mice (three to four mice per group, 8 to 10 wk old) were exposed to cigarette smoke (CS) using the TE-2 cigarette smoking machine (Teague Enterprises, Davis, Calif.). The CS was generated by burning research-grade cigarettes (3R4F; 0.73 mg nicotine per cigarette) purchased from the Tobacco Research Institute (University of Kentucky, Lexington, Ky.) for 5 h/d for 5 d. An average total particulate matter of 150 mg/m³ was recorded in real time during the smoking protocols. The control group of mice was exposed to filtered room air, and all the mice were sacrificed 2 h after the last CS exposure. The human lung tissue samples from Gold stage I (mild, forced expiratory volume [FEV1]% predicted>80%), Gold stage 11 (moderate, FEV1% predicted=˜50-80%), and Gold stage III-IV (severe/very severe, FEV1% predicted <50%) nontumor COPD (with FEV1/forced vital capacity [FVC] ratio of <70%) and Gold stage 0 (at risk) control subjects (procured from Lung Tissue Research Consortium [Bethesda, Md.], National Heart, Lung and Blood Institute, National Institutes of Health) were used for quantification and localization of indicated proteins by immunostaining All the subjects were stable, and Gold I-IV subjects had emphysema. Moreover, one patient in each group (Gold I-IV) had first-degree blood relatives with chronic bronchitis. A detailed description of the human subjects is shown in Table I.

In Vitro and Ex Vivo Experiments.

The macrophages and neutrophils from CFTR^{+/+}and CFTR^{-/-} mice were isolated by intraperitoneal (i.p.) 1 ml injection of 4% thioglycollate broth (Fluka, St. Louis, Mo.). The peritoneal cavity was flushed as indicated after 6 h (32) (neurotrophils) or 4 d (33) (macrophages) with 10 ml RPMI-1640 media (Life Technologies, Carlsbad, Calif.) containing 10% FBS (Life Technologies) and 1% PSA (Life Technologies) (complete RPMI media). The lavage was extracted and centrifuged at 1200 rpm for 8-10 minutes at 4° C. followed by RBC lysis using the LCK lysis buffer (Quality Biologicals, Gaithersburg, Md.). The cells, 3×10⁵ per well were plated in a 6-well plate and cultured overnight in complete RPMI media. The culture supernatants were collected for cytokine ELISAs and myeloperoxidase (MPO) measurements. The spleens were dissected from Cftr^{+/+} and Cftr^{-/-} mice and macerated using the plunger of a 5 ml BD (San Diego, Calif.) syringe. The suspension was subjected to RBC lysis, as described above, and 2×10⁵ splenocytes per well were cultured in a 96-well plate. The cells were treated with 5 or 10 μg/ml Con A for 72 hours. For splenocyte proliferation assay, 20 μl of the Cell Titer 96® AQᵤₑₒᵤₛOne Solution (Promega, Madison, Wis.) was added at 60 hours and the plate was incubated at 37° C., 5% CO₂ for another 12 hours. The optical density (OD) at 490 nm was recorded by a 96-well microplate reader (Molecular Devices, Sunnyvale, Calif.) using a SOFT-MAX-Pro software (Molecular Devices) as a measure of cell proliferation. For immunoblotting, splenocytes (2×10⁶ cells/well in a 6-well plate) were treated with 5 μg/ml ConA for 12 hours and the total protein extract was collected using the M-PER protein lysis buffer and 1× protease inhibitor cocktail in EDTA (Pierce, Rockford, Ill.). The human CF bronchial epithelial cells, CFBE4lo- and CFBE4lo-WT-CFTR were cultured in MEM media supplemented with 10% FBS (Life Technologies) and 1% PSA (Life Technologies). The CFBE4lo-WT-CFTR cells were cultured in the presence of 500 μg/ml Hygromycin B (Invitrogen) to maintain the stable expression of WT-CFTR. For fluorescence or confocal microscopy, equal number of cells were cultured in glass bottom collagen coated 35-mm petri dishes (Mattek Corporation, Ashland, Mass.) and treated for 6 hours with 10 ng/ml Pa-LPS, 5 mM CD and/or 10 ng/ml TNFα (Invitrogen). The CFBE4lo-WT-CFTR cells were treated with PBS or 5 mM CD for 24 hours on a 24-well plate, and IL-8 secretion in the cell supernatants was quantified by sandwich ELISA (R&D Biosystems, Minneapolis, Minn.). The HEK-293 cells were transiently transfected with WT-CFTR and incubated with increasing doses (0, 40, 80, 120 and 160 μg/ml) of cigarette smoke extract (CSE, Murty Pharmaceuticals) for 12 hours. The total protein lysate cell lysate from these samples was extracted as described above and levels of mature (C form) and immature (B form) of CFTR were quantified by Western blotting. The lipid-rafts were isolated from CFBE4lo-WT-CFTR and CFBE4lo-cells treated with PBS, Pa-LPS (10 ng/ml) or Fumonisin B1 (50 μM) for 24 hours. For the ΔTRL/WT-CFTR experiments, HEK-293 cells were transiently transfected with pEGFP-WT-CFTR or pEGFP-ΔTRL-CFTR (a gift from Dr. William B. Guggino, Johns Hopkins University) constructs (24) using Lipofectamine 2000 (Invitrogen) for a total of 48 h. The cells were treated with 100 μg/ml CSE for the final 12 h and analyzed by flow cytometry. For LPS binding experiments, HEK-293 cells were similarly transfected with WT or ΔTRL constructs and incubated with FITC-labeled Escherichia coli LPS (Molecular Probes, Carlsbad, Calif.) for the final 3 h and analyzed by flow cytometry without permeabilizing the cells. The same set of transfections was also performed with or without 50 ng/ml TNF-α treatment for 6 h, and lipid-raft proteins were isolated to detect CFTR expression by Western blotting.

Immunofluorescence Microscopy and Flow Cytometry.

The longitudinal tissue sections from murine or human lungs or CFBE41o- and CFBE41o-WT-CFTR cells were immunostained with the primary Abs (1:50 to 1:200 dilution) for CFTR (rabbit polyclonal; Santa Cruz Biotechnology, Santa Cruz, Calif.), ceramide (mouse monoclonal; Alexis Biochemicals, Plymouth Meeting, Pa.), Foxp3 (rabbit polyclonal; Santa Cruz Biotechnology), NF-κB (rabbit polyclonal; Santa Cruz Biotechnology), zona occludens (ZO)-1 (rabbit polyclonal; Santa Cruz Biotechnology), ZO-2 (goat polyclonal; Santa Cruz Biotechnology), and neutrophil marker NIMP-R14 (rat monoclonal; Abcam, Cambridge, Mass.) followed by the secondary Abs (1:200 dilution), using our previously described protocol (34). The secondary Abs used were goat anti-rabbit IgG FITC (Santa Cruz Biotechnology), goat anti-rat IgG (H+L) R-PE, goat anti-mouse IgG/IgM (H+L), Alexa Fluor 488, donkey anti-goat Alexa Fluor 488 (Invitrogen), donkey anti-mouse Dylight 594, donkey anti-rat Dylight 488, and donkey anti-goat Dylight 594 (Jackson ImmunoResearch, West Grove, Pa.). Nuclei were detected by Hoechst (Invitrogen) staining, and H&E was used to evaluate lung morphology and inflammatory state. Images were captured by an Axiovert 200 Carl Zeiss (Thornwood, N.Y.) Fluorescence microscope using the Zeiss Axiocam HRC camera and Axiovision software. The membrane localization of ZO-1 and ceramide in CFBE41o-WT-CFTR cells was detected by confocal microscopy. The staining protocol for confocal microscopy was similar to the fluorescence staining protocol. The images were captured using a Zeiss LSM 510 Meta confocal microscope and analyzed by Zeiss LSM Image Browser software. All fluorescent and confocal images were captured at room temperature with oil (×340 confocal and ×363 fluorescence) and air (×320 and ×340 fluorescence) as the imaging medium. The magnifications for the confocal and fluorescence microscopes were EC Plan-Neo Fluar (×40/1.3 oil, confocal), LD Plan-Achroplan (×20/0.40 Korr Phz, fluorescence), LD Plan-Neo Fluar (×40/×0.6 Phz Korr, fluorescence), and LD Plan-Achromat (×63/1.4 oil), respectively, with ×1.6 optivar. Splenocytes were isolated from Cftr^{+/+} and Cftr^{-/-} mice for flow cytometry, and nonspecific Ab binding was blocked by incubating them with either donkey or goat serum (1:10; Sigma). Cells were washed once in FACS buffer (2% FBS in PBS) and double stained with CD4-PE (rat monoclonal; Santa Cruz Biotechnology), and CFTR or intracellular Foxp3 primary Abs followed by anti-rabbit FITC secondary Ab or stained with CD4-PE followed by intracellular IFN-γ-FITC (rat polyclonal; Invitrogen). The macrophages and neutrophils were double stained with the respective cell surface markers, Mac 3 (rat monoclonal; Abcam) or NIMP-R14 (rat monoclonal; Abcam) and ceramide or ZO-1 primary Abs followed by anti-rat R-PE, anti-mouse Alexa Fluor 488, or anti-rabbit FITC secondary Abs. The cells were stained and washed two times in FACS buffer and resuspended in 0.1% paraformaldehyde (USB, Cleveland, Ohio). Appropriate secondary Ab controls were used in all the flow cytometry experiments. The Fix & Permcell Permeabilization kit (Invitrogen) was used for IFN-γ, Foxp3, and ceramide intracellular staining following the manufacturer's protocol. The cells were acquired using the BD FACSCaliber instrument, and analysis was done with the BD Cell Quest Pro software.

ELISA, MPO Activity, and Reporter Assay.

The BALF and cell culture supernatants (n=3-5) were quantified in triplicate for mouse IL-6, IL-1β, or human IL-8 using ELISA kits (R&D Systems, or eBioscience, San Diego, Calif.) following the manufacturer's instructions. MPO levels in neutrophil culture supernatant or mouse BALF were similarly quantified using the MPO ELISA kit (Hycult Biotechnology, Uden, The Netherlands). For reporter assays, CFBE41o-WT-CFTR or CFBE41o-cells were transfected with NF-KB firefly luciferase promoter (pGL2) and renilla luciferase (pRLTK) control using Lipofectamine 2000 (Invitrogen). Renilla luciferase was used as an internal control for normalization of DNA and transfection efficiency of reporter constructs. Cells were induced with 10 ng/ml TNF-α and/or 50 mM FB1 for 12 h, and luciferase activities were measured after overnight treatment using the Dual-Luciferase Reporter Assay System (Promega) as described previously (28). Data were normalized with internal renilla luciferase control for each sample, and the changes in reporter activities with CFTR overexpression were calculated.

Immunoblotting and Lipid-Raft Isolation.

Splenocytes from Cftr^{+/+} and Cftr^{-/-} mice were isolated and stimulated with 5 μg/ml Con A for 12 h. Cells were washed in PBS, and total protein was isolated using the 1×M-PER Mammalian protein extraction reagent (Pierce) supplemented with protease inhibitor mixture (Sigma). The protein lysate was immunoblotted for Foxp3 primary (Santa Cruz Biotechnology) or β-actin (Sigma) loading control and anti-rabbit IgG HRP secondary Abs (Amersham, Piscataway, N.J.) and developed using the Super Signal West Pico Chemiluminescent Substrate kit (Pierce). Similarly, the total cell lysates from HEK-293 cells transiently transfected with the WT-CFTR and treated with increasing doses of CSE were immunoblotted with CFTR (Cell Signaling Technologies, Danvers, Mass.) or β-actin (Sigma) loading control and anti-rabbit or anti-mouse-HRP Ab, respectively. The mouse lung tissue from air and CS exposed mice was homogenized in cold tissue lysis buffer (T-PER; Pierce) supplemented with protease inhibitor mixture. The lung lysate was immunoprecipitated with CFTR 169 Ab (rabbit polyclonal), followed by Western blot with CFTR (M3A7) Ab (Abcam). For lipid-raft isolation, CFBE41o- and CFBE41o-WT-CFTR cells were plated in a 25 cm² tissue culture flask and treated with Pa-LPS (10 ng/ml) and/or FB1 (50 mM) for 24 h. The cells were washed with cold PBS, and raft proteins were isolated using the Signal Protein Isolation kit (G Biosciences, Maryland Heights, Mo.). The lung tissue from air and CS exposed mice was similarly harvested in signal protein extraction (SPE) buffer-I and subjected to raft isolation as described below. Briefly, cells or lung tissue were resuspended in SPE buffer-I and sonicated for 10 s to disrupt the cells or tissue. Total protein was quantified in each sample, and equal amount of protein (cells, 300 μg; and lung tissue, 500 μg) was used to purify the raft fraction. The SPE buffer-II was added followed by incubation on ice for 15 min with intermittent vortexing. The lysate was centrifuged at 20,000×g for 15 min and the supernatant discarded. The pellet containing signal proteins was solubilized in adequate amount of focus protein solubilization buffer and used for immunoblotting of ZO-2 (Santa Cruz Biotechnology, goat primary and anti-goat IgG HRP) and a-actin (Sigma, rabbit primary and anti-rabbit IgG HRP). The raft protein from mouse lungs or HEK-293 cells was immunoblotted with CFTR 570 Ab (mouse polyclonal Ab; procured from University of North Carolina, Chapel Hill and Cystic Fibrosis Foundation Therapeutics under a material transfer agreement).

Statistical Analysis.

Data are represented as the mean±SEM of at least three experiments, and Student t test and ANOVA were used to determine the statistical significance. The murine and human microscopy data were analyzed by densitometry (MATLAB R2009b; Mathworks, Natick, Mass.) followed by Spearman's correlation coefficient analysis to calculate the significance among the indicated groups.

### Example 1

### CFTR Regulates Innate and Adaptive Immune Response

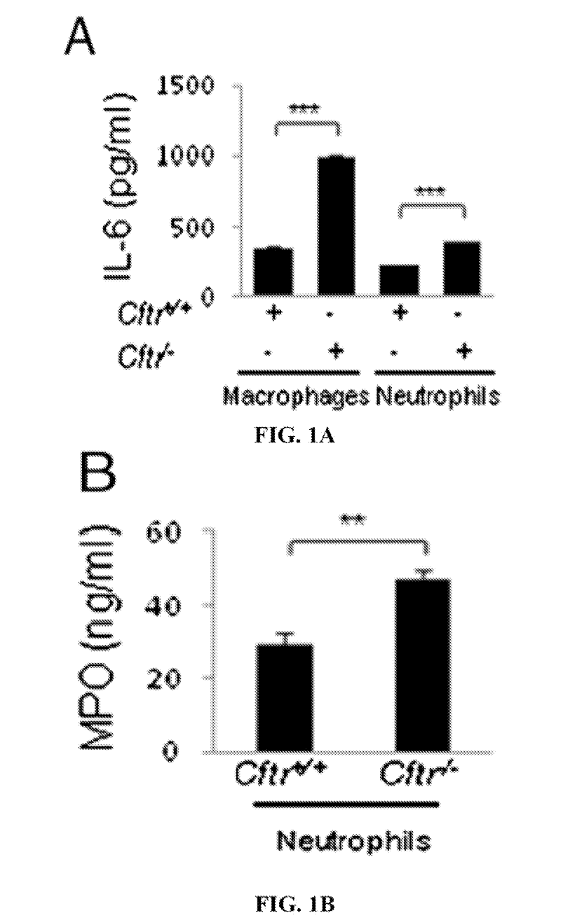

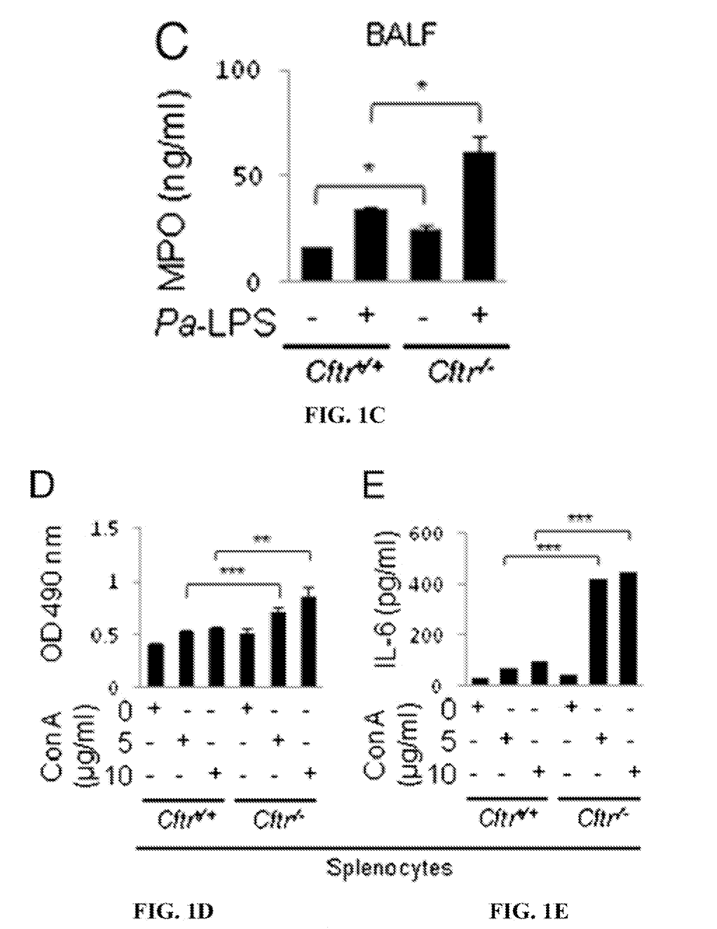

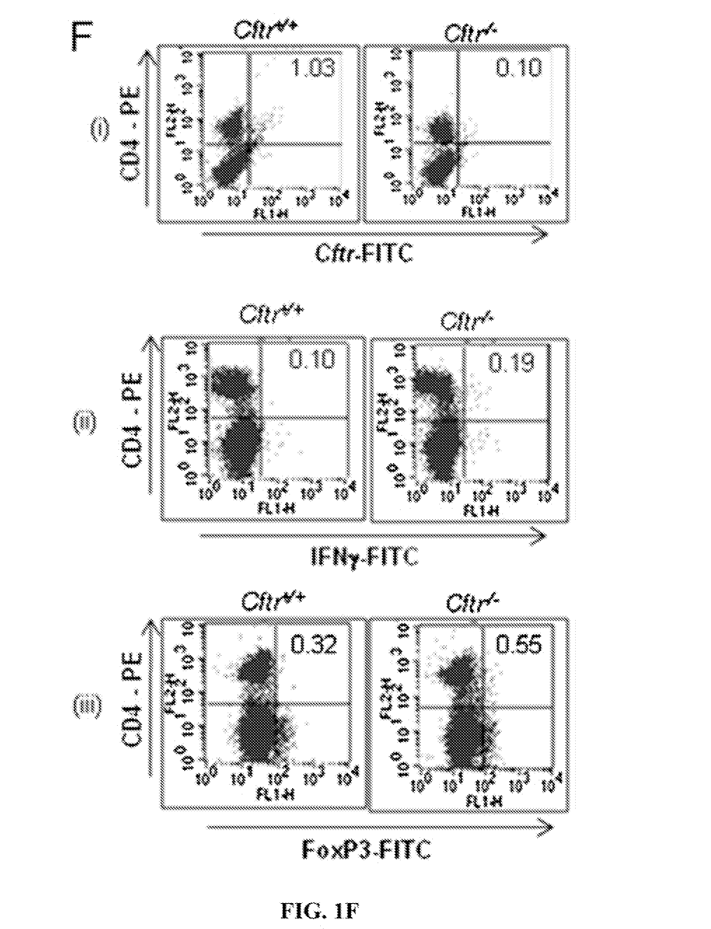

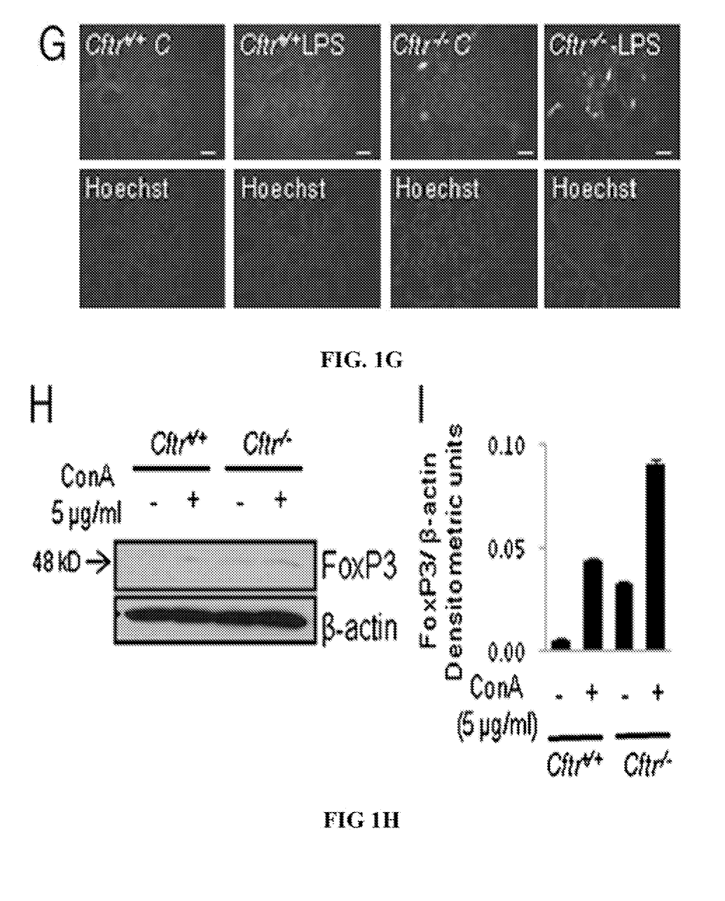

To confirm and expand the hypothesis that functional CFTR is a critical regulator of inflammatory signaling (28), the immune profile of the gut-corrected Cftr^{-/-} mice was compared with that of the Cftr^{+/+} mice. The constitutive levels of proinflammatory cytokine IL-6 were quantified ex vivo in peritoneal macrophages and neutrophils isolated from Cftr^{+/+} and Cftr^{-/-} mice (n=3) and significantly (p , 0.001) higher basal IL-6 levels in Cftr^{-/-} were found compared with that in the Cftr^{+/+} (FIG. 1A). A significant increase (p , 0.01) was also found in constitutive neutrophil-MPO levels (FIG. 1B) in Cftr^{-/-} compared with those in the Cftr^{+/+}, which is indicative of the activated state of neutrophils in the absence of CFTR. We confirmed this in vivo using the murine model and observed a significant increase (p , 0.05) in basal and Pa-LPS-induced MPO levels in BALF of Cftr^{-/-} mice compared with those in the Cftr^{+/+} mice (FIG. 1C). To test the outcome of CFTR deficiency on the adaptive immune response, we quantified differences in cell proliferation and IL-6 secretion in splenocytes from Cftr^{+/+} and Cftr^{-/-} mice. We did not find a significant difference in the nonactivated splenocytes, but Con A induced a significantly higher (**p , 0.01, ***p , 0.001) splenocyte proliferation and IL-6 secretion in Cftr^{-/-} compared with that in Cftr^{+/+}(FIG. 1D, 1E). We confirmed that CFTR is expressed on murine splenocytes (FIG. 1Fi). The CFTR-deficient splenocytes demonstrate higher numbers of CD4+IFN-g+ T cells (FIG. 1Fii) supporting the notion that the absence of CFTR results in a constitutive hyperinflammatory state by inducing the proinflammatory response. In addition, prevalence of regulatory T cells is reported in the hyperinflammatory COPD lungs (35). We compared the expression of Foxp3 in Cftr^{+/+} and Cftr^{-/-} mice and found constitutively higher numbers of CD4+Foxp3+ splenocytes in the Cftr^{-/-} (0.55%) compared with that in the Cftr^{+/+}(0.32%) (FIG. 1Fiii). We also confirmed this by Foxp3 immunostaining and Western blotting in lung sections and splenocytes, respectively (FIG. 1G, 1H). The data substantiate the previous observations (28, 36-40) and strongly suggest that CFTR is a critical regulator of both innate and adaptive immune responses.

### Example 2

### CFTR Expression in Inflammatory Cells Inversely Correlates with the Levels of Ceramide and Lipid-Raft Marker (ZO-1)

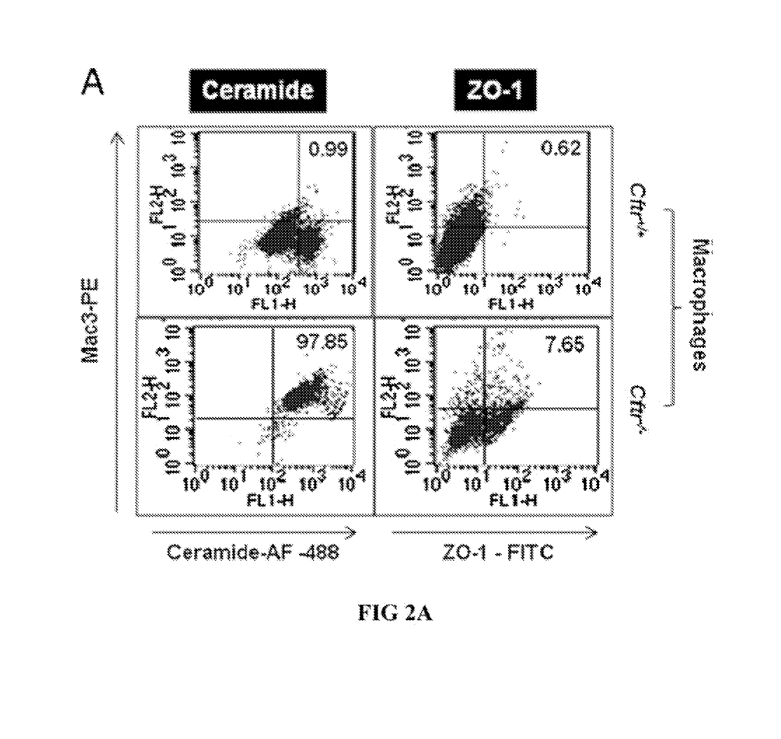

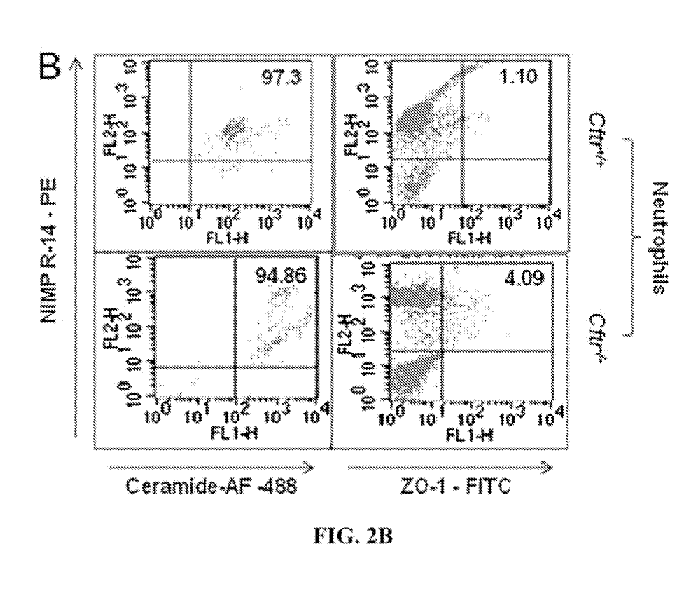

Ceramide is a critical regulator of inflammatory and apoptotic signaling (20) and mediates these processes in lung injury (41), asthma (21), emphysema, COPD (20), and CF (7). Moreover, CFTR is present in the lipid-rafts (27, 42), and its role in regulating TNF-R1 and lipid-raft signaling has been examined previously (27). We tested the hypothesis that CFTR may be regulating inflammatory signaling via ceramide by inhibiting the formation of membrane and lipid-raft platforms, which would hamper proper clustering of signaling receptor complexes on the plasma membrane. Evidence from previous studies (7) and our data show that macrophages from Cftr^{-/-} mice have significantly higher ceramide levels compared with those of the Cftr^{+/+} mice (FIG. 2A, left panel), which concurs with increased expression of lipid-raft marker ZO-1 (FIG. 2A, right panel). Although the Cftr^{-/-} neutrophils show a similar increase in ZO-1 expression, ceramide levels remain unchanged (FIG. 2B). We speculate that other mechanisms may be involved in constitutive increase of neutrophil (MPO) activity in the absence of CFTR (14, 17). Our data indicate a mechanism by which

CFTR regulates lipid-raft signaling and inflammatory cell function (s). The constitutive defect in the absence of CFTR compromises the ability of these inflammatory cells to respond to infection or injury resulting in pathogenesis of chronic lung disease.

### Example 3

### CFTR Regulates Membrane-Ceramide Signaling and Pathogenesis of Chronic Emphysema

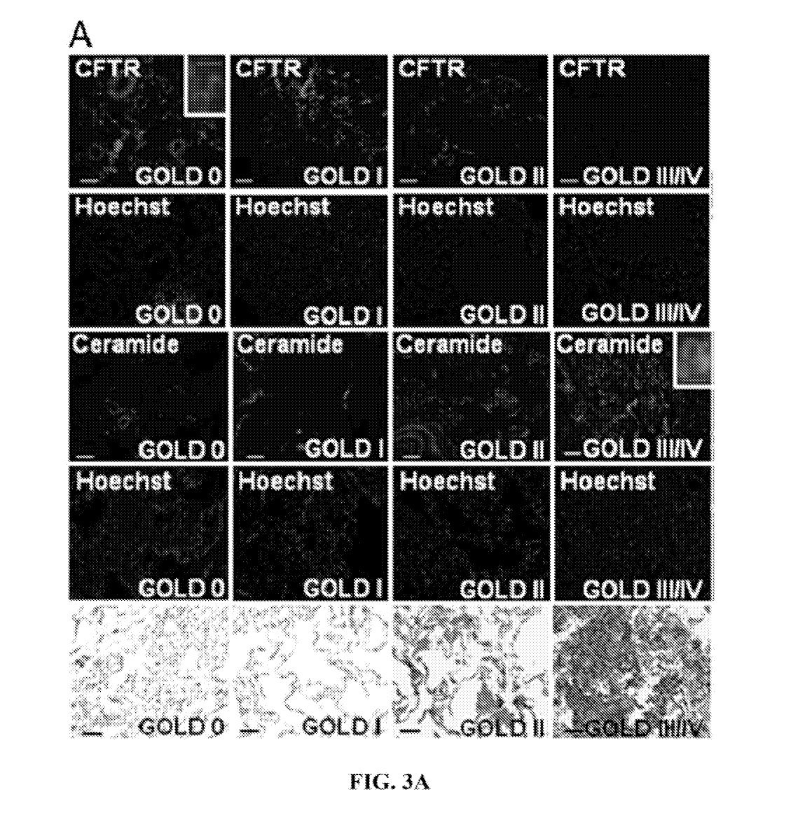

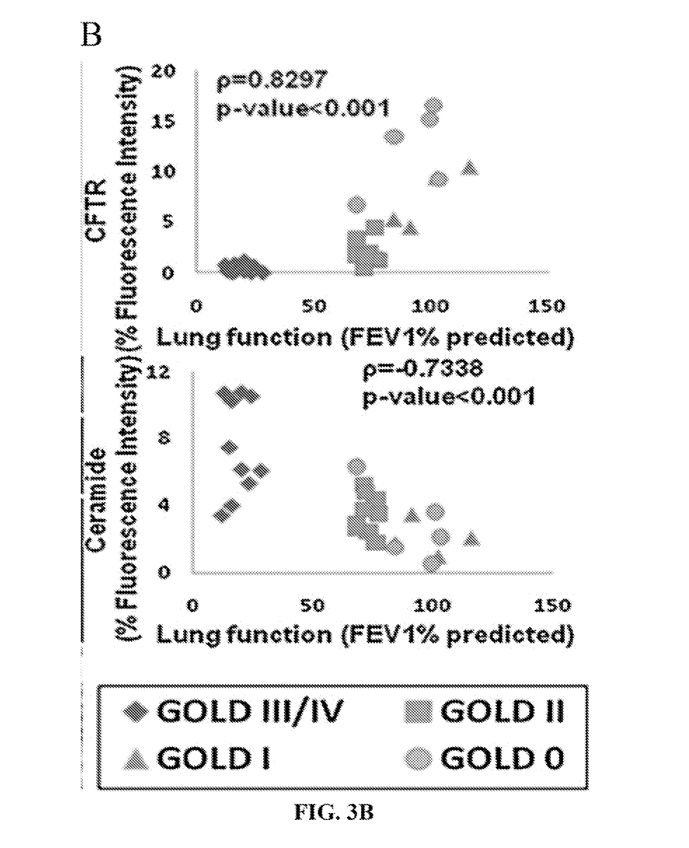

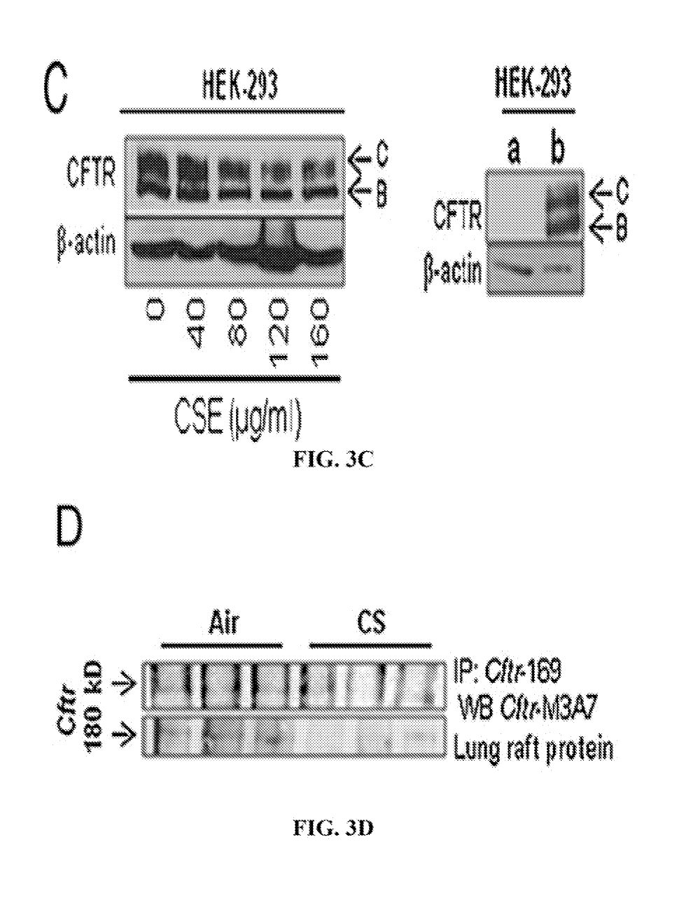

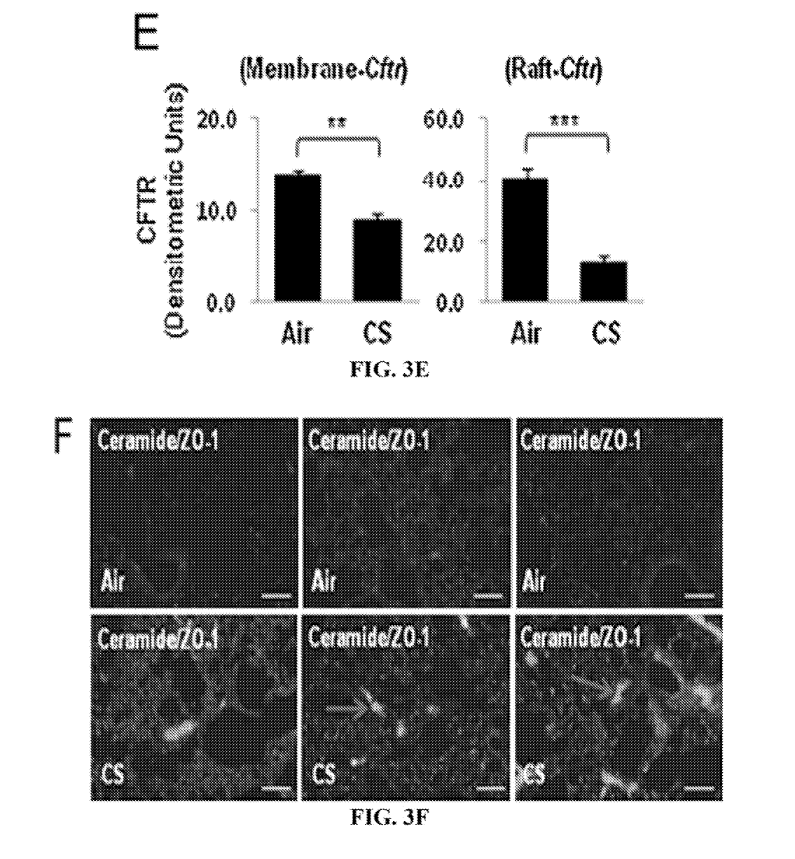

Ceramide upregulation was recently correlated with emphysema (20), and it is known that CFTR deficiency leads to increased ceramide accumulation and lung injury (7). We verified this observation in lung sections from control (Gold 0, at risk) and COPD (Gold I, mild; II, moderate; and III-IV, severe and very severe emphysema) human subjects (Table I) and found that CFTR expression significantly decreases with disease severity while ceramide levels increase (FIG. 3A, 3B, p , 0.001). Although CFTR is not completely absent in severe COPD lungs, its expression is significantly downregulated. We anticipate this as an outcome of lung injury. These data imply that lipid-raft localization of CFTR (FIG. 3A, inset) controls ceramide accumulation and possibly severity of emphysema. We confirmed our findings in HEK-293 cells transfected withWT-CFTR and show that CSE treatment decreased cell surface expression of CFTR (mature, band C) in a dosedependent manner (FIG. 3C, left panel). The non-transfected HEK-293 cells do not show the CFTR at this Ab concentration (FIG. 3C, right panel). Extending our findings in the murine model (C57BL/6 mice), we found that acute CS exposure (5 h/d for 5 d) diminishedCFTR expression both in the mouse lung lysate (FIG. 3D, upper panel, and FIG. 3E, left panel, p , 0.01) and in the purified lipid-raft fraction (FIG. 3D, lower panel, and FIG. 3E, right panel, p , 0.001). Moreover, we also demonstrate that lungs of CSexposed mice have significantly (p=0.004, r=0.9316) increased ceramide accumulation that is colocalized with ZO-1 (FIG. 3F), which implies that CS-mediated decrease in CFTR expression results in lipid-raft ceramide accumulation. Therefore, in accord with our previous observation (28), the current data verify that decreased cell surface and lipid-raft expression of CFTR correlates with the increased inflammation and emphysema (FIG. 3A, H&E staining, bottom panel).

TABLE I
Patient Characteristics

| | Gold 0 | Gold I | Gold II | Gold III-IV |
|---|---|---|---|---|
| Parameters | (At Risk) | (Mild) | (Moderate) | (Severe) |
| Avg. age (y), | 70.4 ± 8.3 | 69.5 ± 5.97 | 70.9 ± 9.8 | 53.3 ± 6.11 |
| mean ± SD | | | | |
| Sex (n) | M(2), F(3) | M(3), F(1) | M(5), F(4) | M(1), F(9) |
| Smoking | Ever (3) | Current (1) | Current (0) | Current (0) |
| Status (n) | Never (2) | Ever (2) | Ever (8) | Ever (10) |
| | | Never (1) | Never (1) | Never (0) |
| Pack/year, avg. | 33.6 ± 47.4 | 27.7 ± 27.3 | 45.4 ± 36.5 | 37.4 ± 37.3 |
| FEV1% | 91 ± 14.8 | 98.2 ± 13.9 | 72.4 ± 3.35 | 18.7 ± 5.2 |
| predicted, | | | | |
| mean ± SD | | | | |

### Example 4

### CFTR Expression Regulates Ceramide Signaling in Lung Injury

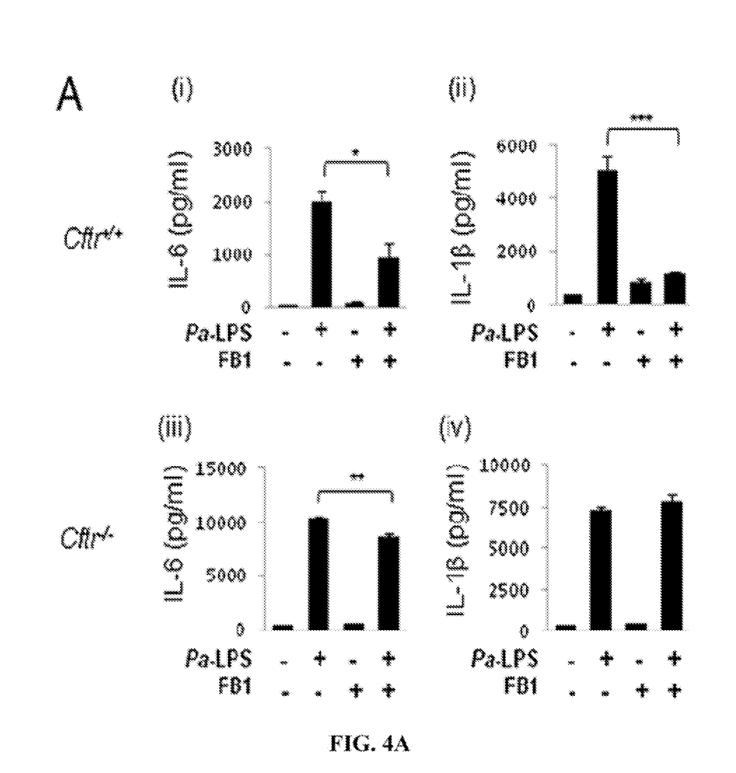

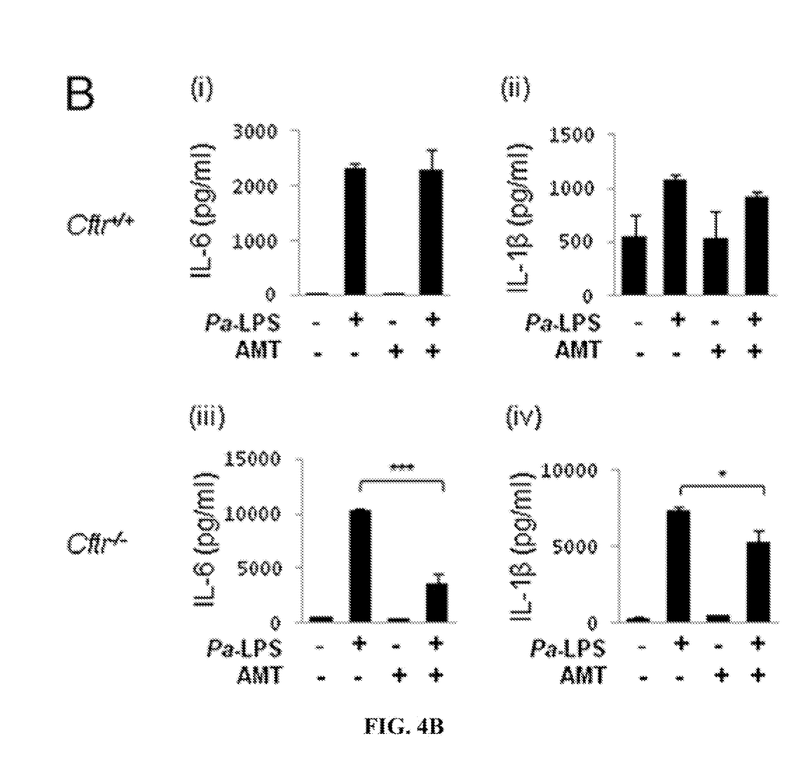

To verify whether CFTR regulates ceramide signaling and outcome of lung injury, we used the Pa-LPS-induced mouse model of lung injury. We treated Cftr^{+/+} and Cftr^{-/-} mice with 20 mg/mouse Pa-LPS i.t. for 12 h, followed by either FB1 or AMT (50 mg/mouse) for another 24 h. We inhibited either the de novo ceramide synthesis (FB1) or membrane-ceramide release (AMT), as they have been shown to mediate the pathogenesis of emphysema and CF lung disease, respectively (7, 20). We measured BALF cytokines IL-6 and IL-1b in all the groups as a marker of Pa-LPS-induced proinflammatory insult and the efficacy of the drugs. We found that inhibition of de novo ceramide synthesis by FB1 in Cftr^{+/+} mice shows a 2-fold reduction (p , 0.05) in the Pa-LPS-induced IL-6 levels (FIG. 4Ai) and a very significant decrease (p , 0.001) in IL-1b secretion (FIG. 4A ii). In the absence of Cftr (Cftr^{-/-} mice), FB1 treatment decreased Pa-LPS-induced IL-6 (FIG. 4A iii), but the magnitude of rescue was not as efficient as that in Cftr^{-/-} mice. In addition, IL-1b levels were unaltered by FB1 treatment in the Cftr^{-/-} mice (FIG. 4A iv). This was also verified by immunostaining of lung sections from these mice for ceramide, NF-kB, and neutrophil marker NIMP-R14 (Supplemental FIG. 1A, 1B). In contrast, inhibition of membrane-ceramide release by AMT was unable to rescue Pa-LPS-induced IL-6 or IL-1b secretion in Cftr^{+/+} mice (FIG. 4Bi, 4Bii), whereas inhibition of membraneceramide in the Cftr^{-/-} mice showed a significant decrease (p , 0.05) in Pa-LPS-induced IL-6 and IL-1b levels (FIG. 4Biii, 4Biv). The ceramide, NF-kB, and NIMP-R14 immunostaining of murine lungs verified these findings (Supplemental FIG. 2A, 2B). Our data concur with findings of Teichgra″ber et al. (7) who showed that normalization of acid sphingomyelinase (Asm) levels by AMT treatment or partial genetic deficiency reduced pulmonary ceramide levels that protected Cftr-deficient mice from P. aeruginosa infection. Our results indicate that inhibition of de novo ceramide synthesis (not the release) by FB1 may be effective in disease states with low CFTR expression like emphysema and lung injury but not in total absence of apical or lipid-raft CFTR, for instance in DF508-cystic fibrosis (DF508-CF), where phenylalanine mutation impairs the folding and trafficking of CFTR to the cell surface. In contrast, inhibition of Asm activity or membrane-ceramide release by AMT has potential application as a more effective drug treatment for DF508-CF but may not be effectual in treating lung injury and emphysema.

### Example 5

### CFTR Expression Negatively Regulates Membrane-Ceramide and Lipid-Raft Signaling

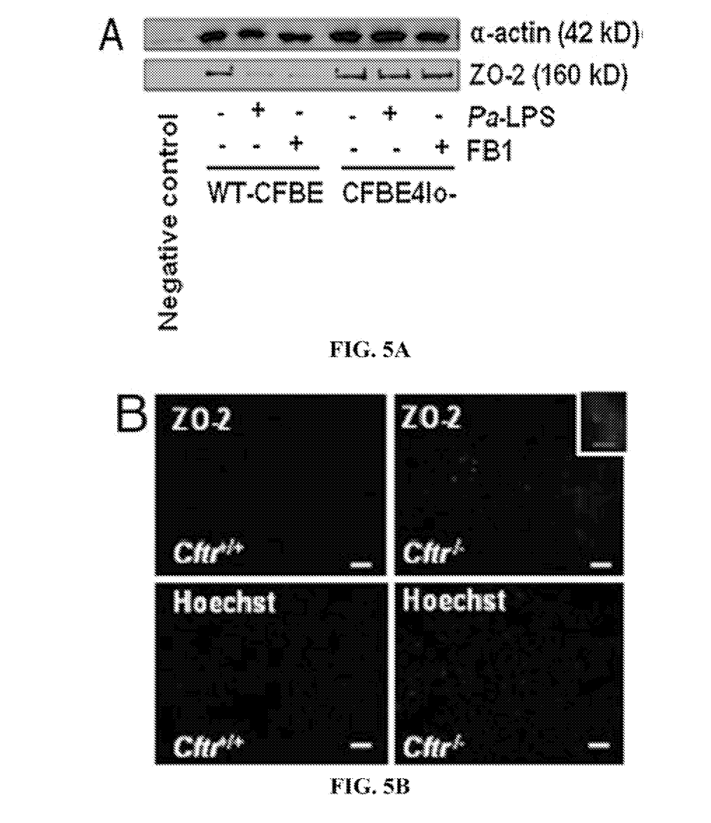

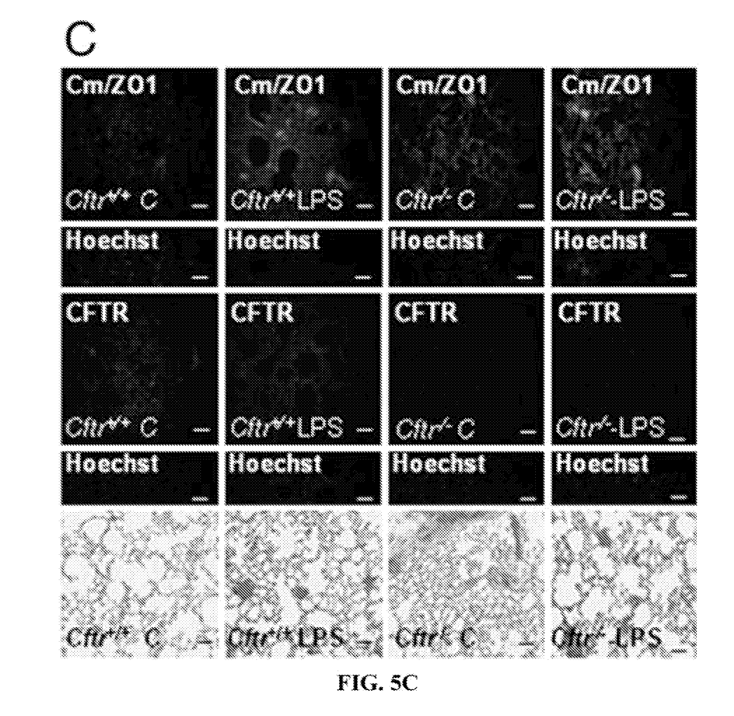

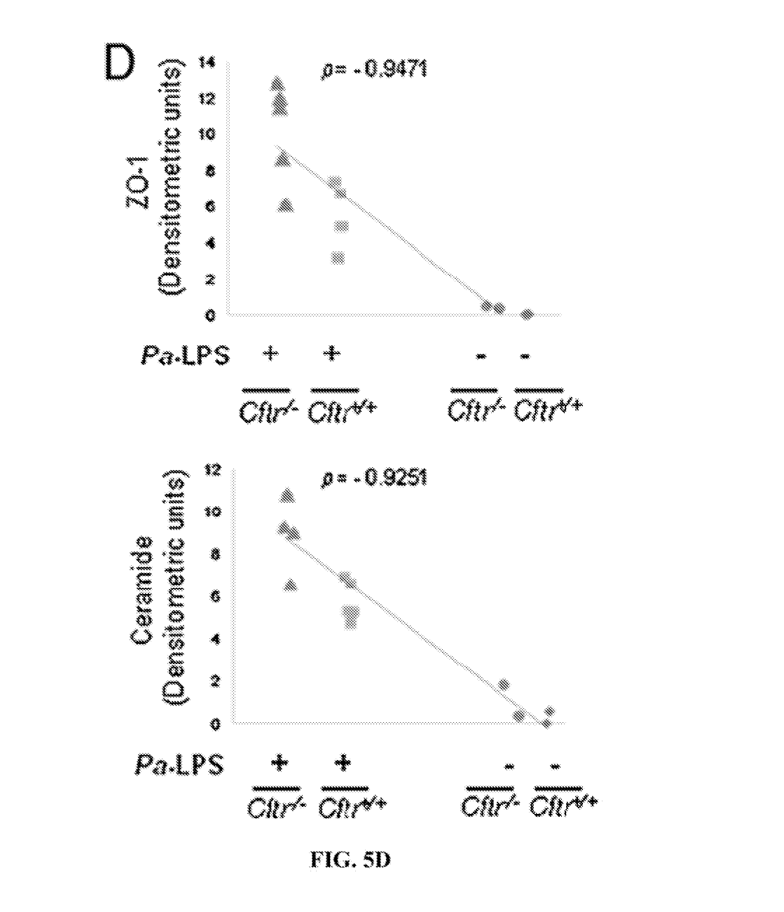

To elucidate the mechanism by which CFTR regulates lipid-raft signaling, we quantified the expression of tight junction protein ZO-2 in purified raft-protein extract from CFBE41o2WT-CFTR and CFBE41o2 cells with or without Pa-LPS or FB1. We found that ZO-2 expression was downregulated by Pa-LPS or FB1, only in the presence of WT-CFTR (FIG. 5A). It is possible that Pa-LPS may induce more recruitment of WT-CFTR to the raft (41, 43), which in turn inhibits raft formation (low ZO-2). FB1 is also able to modulate ZO-2 expression by an unknown mechanism that needs further investigation. Moreover, in the absence of functional CFTR in CFBE41o2 cells, we observed higher basal expression of ZO-2 compared with that in CFBE41o2WT-CFTR cells. We also observed that neither Pa-LPS nor FB1 is able to modulate ZO-2 expression in these cells (FIG. 5A). To confirm these data, we stained the lung sections from Cftr^{+/+} and Cftr^{-/-} mice with ZO-2 and found a constitutively higher ZO-2 expression in the Cftr-deficient mouse lungs (FIG. 5B). We also tested another marker of tight junctions, ZO-1, and analyzed it by co-immunostaining with ceramide using the lung sections from Cftr^{+/+} and Cftr^{-/-} mice that were treated with Pa-LPS or PBS. We found a constitutive increase in ceramide levels in the Cftr^{-/-} mice lungs compared with that in the Cftr^{+/+} mice, which was significantly enhanced by Pa-LPS treatment. Moreover, ceramide was colocalized with ZO-1 indicating its presence in the membrane lipid-rafts. (FIG. 5C, 5D).

### Example 6

### Lack of PDZ Binding Domain Modulates CFTR-Dependent Ceramide Accumulation

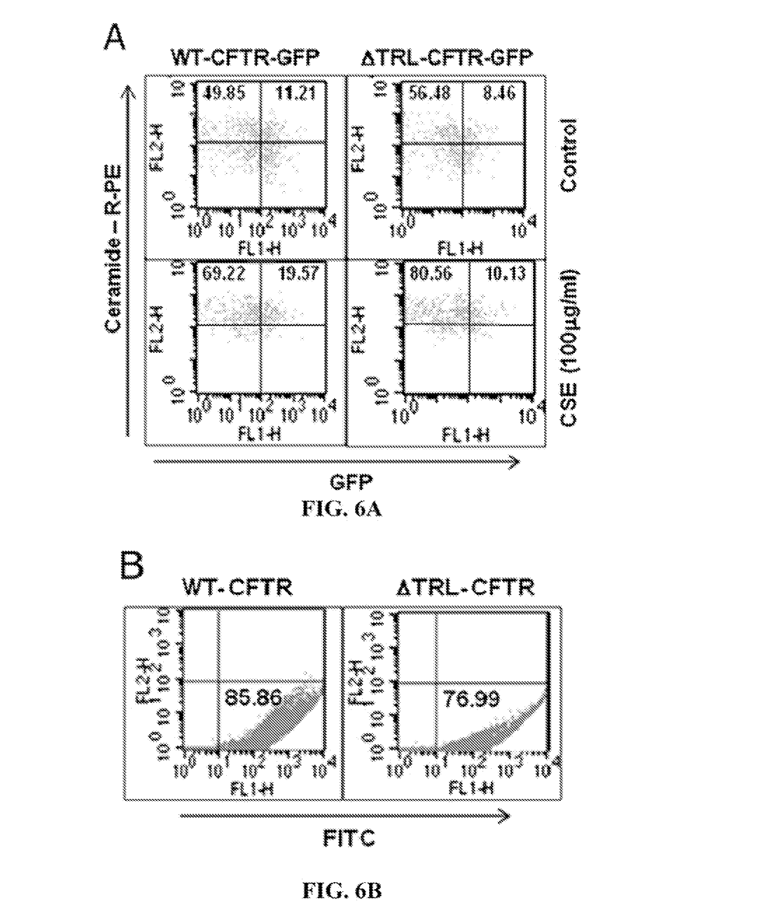

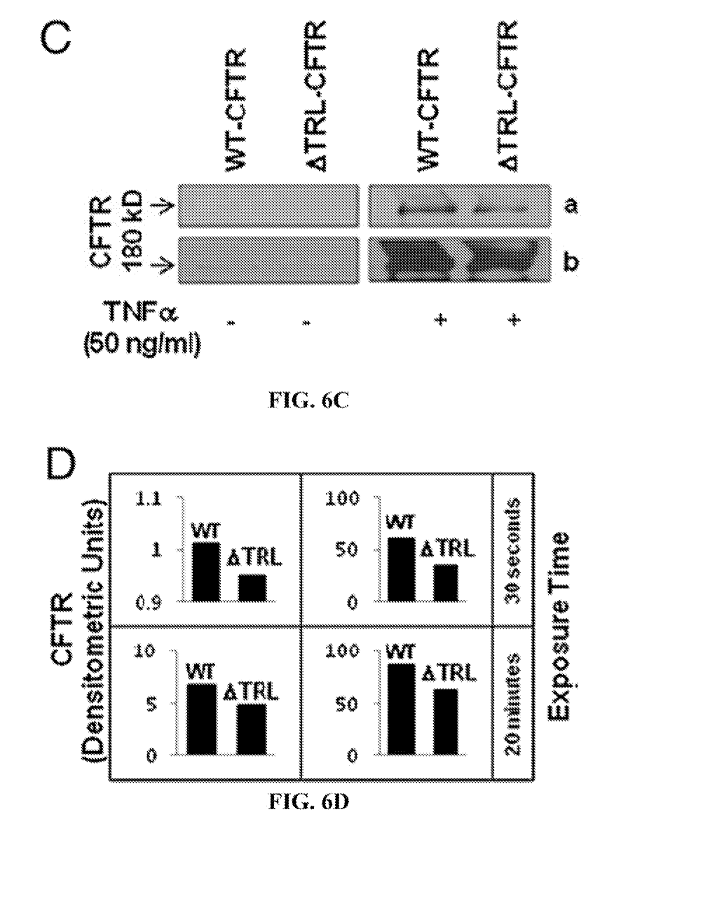

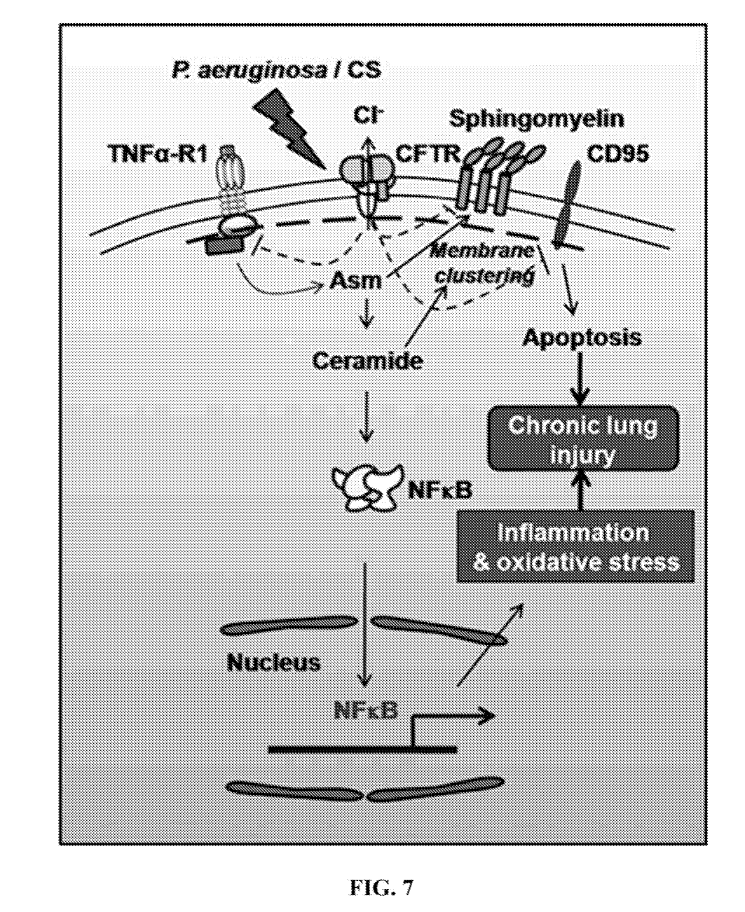

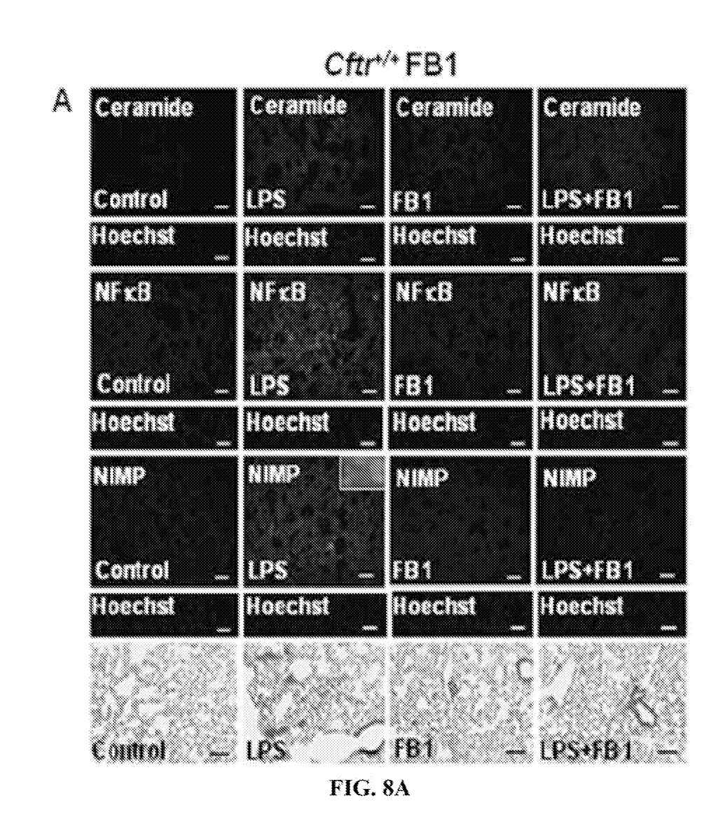

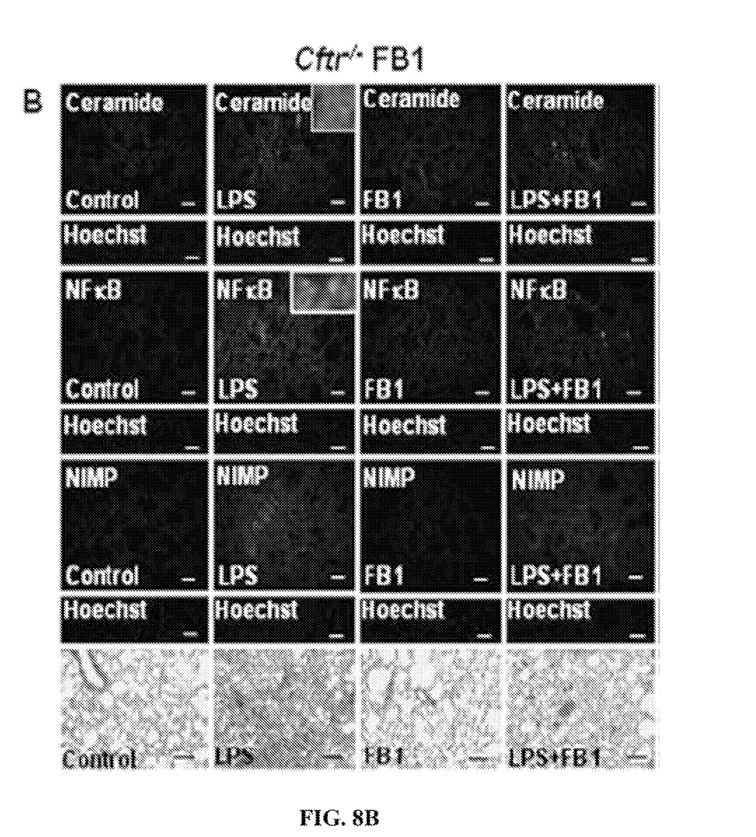

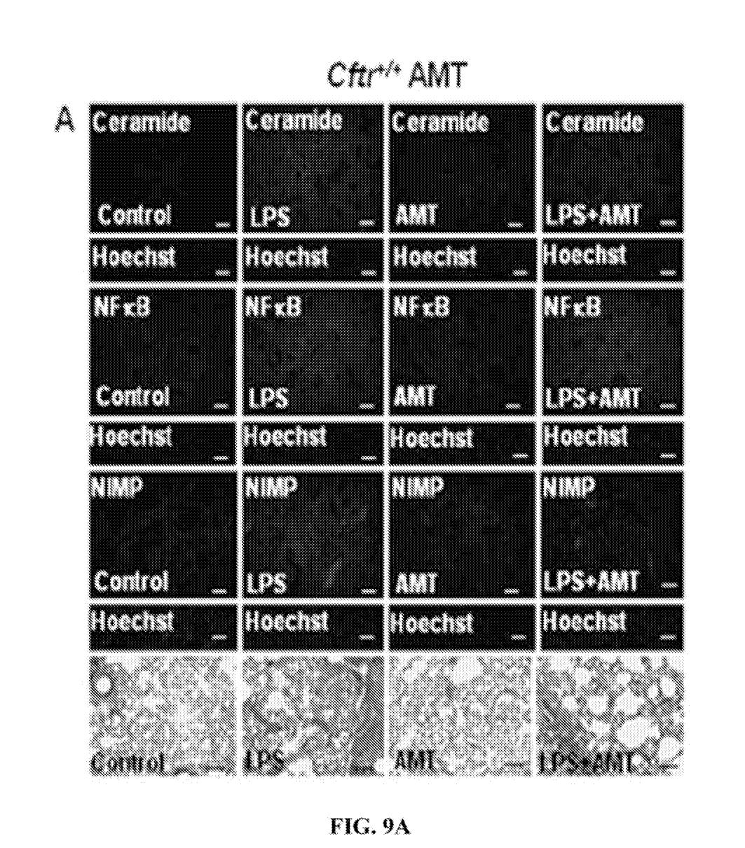

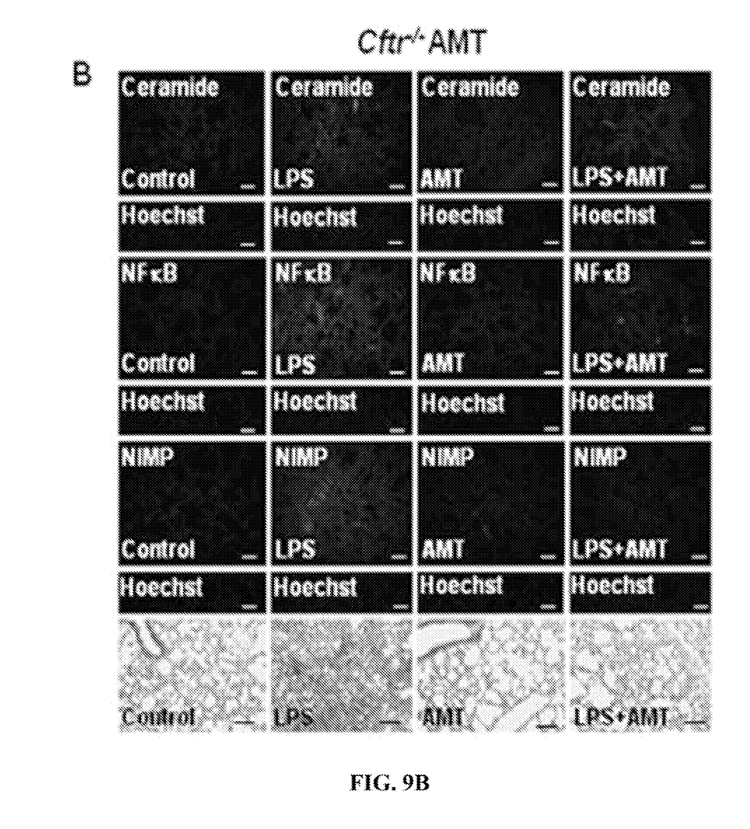

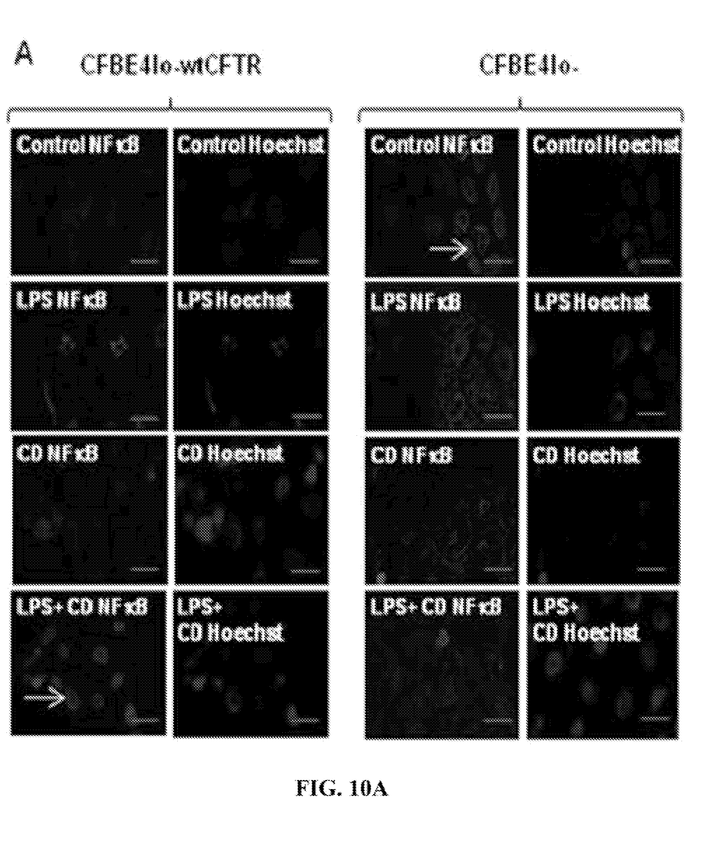

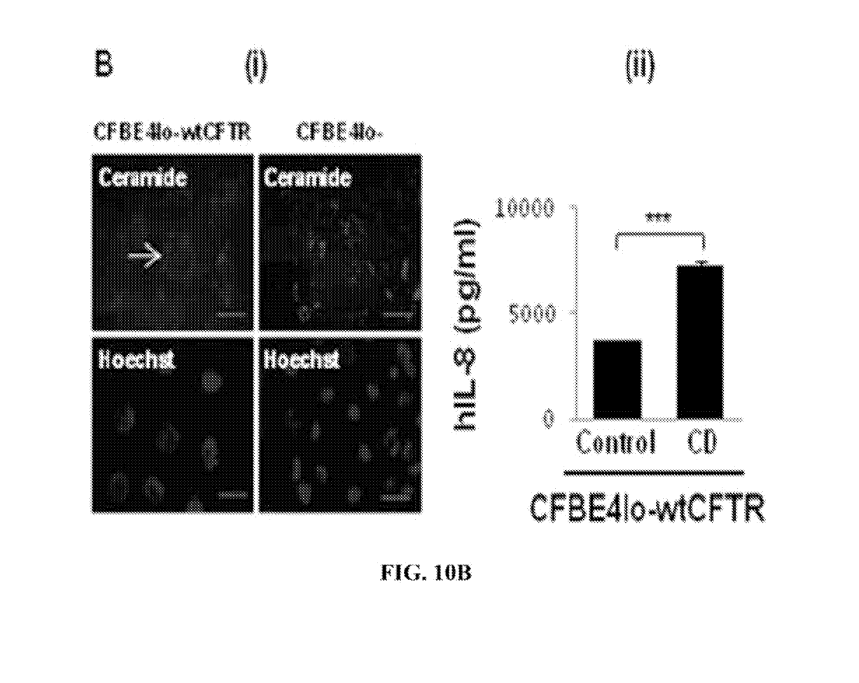

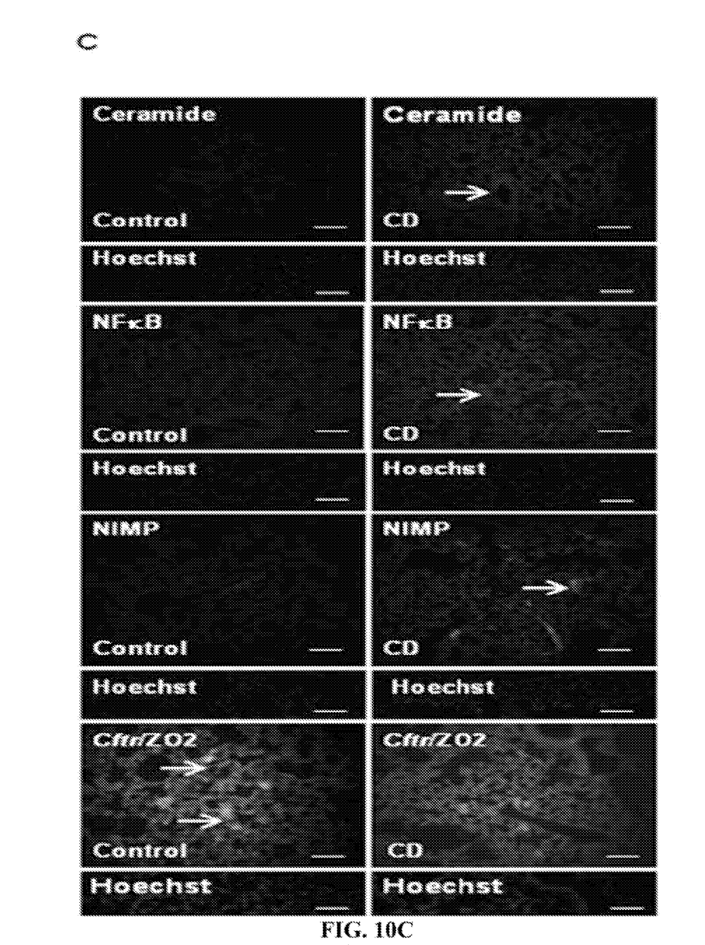

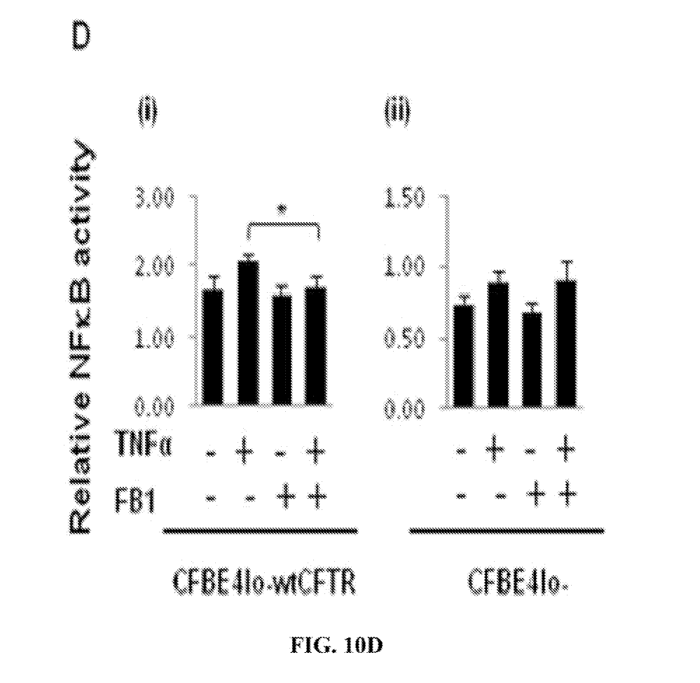

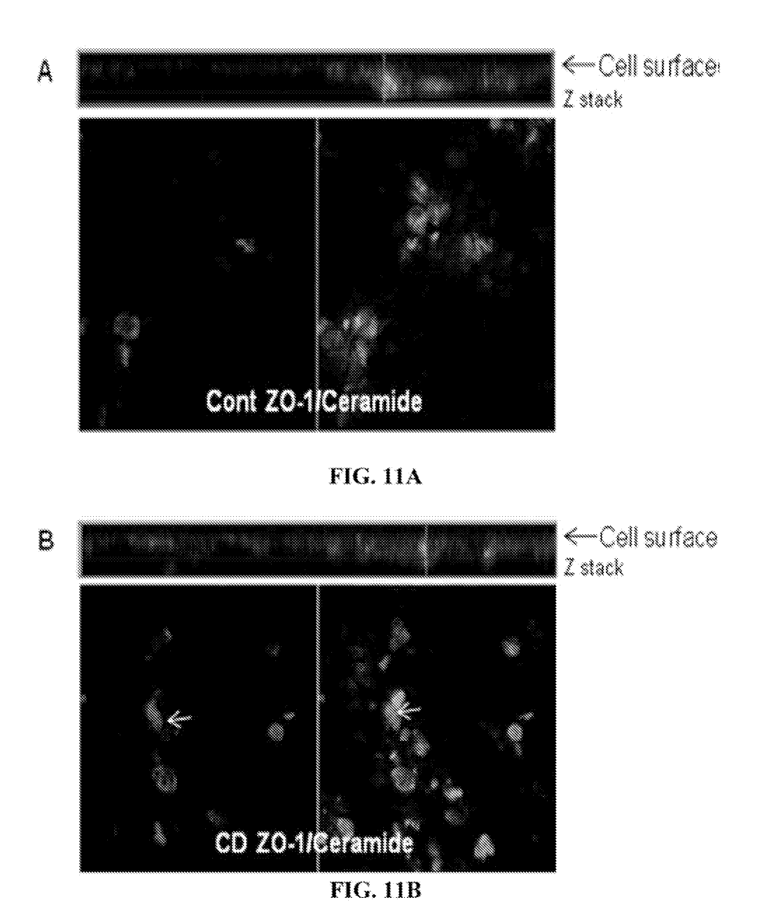

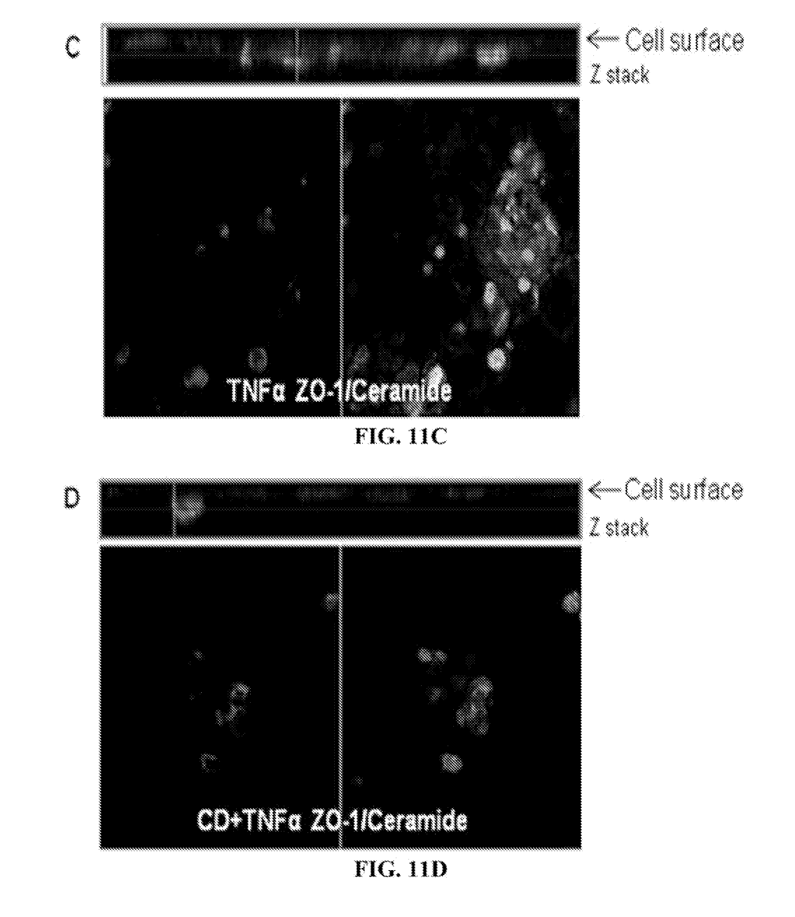

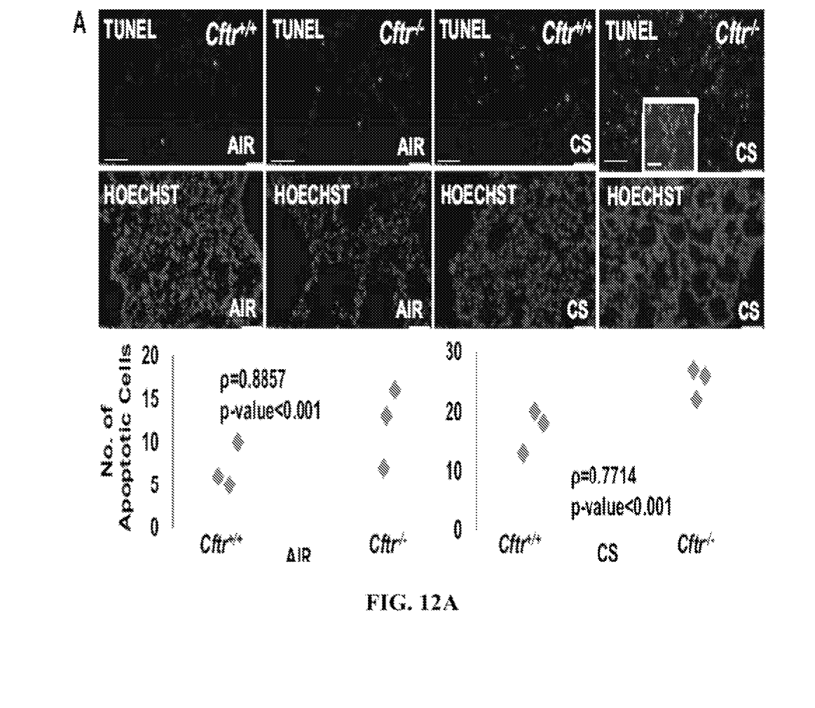

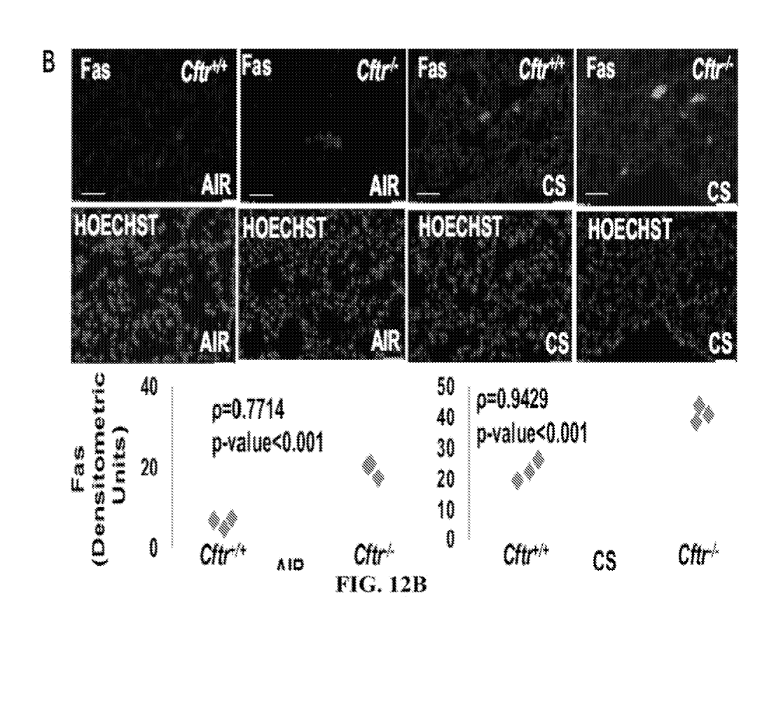

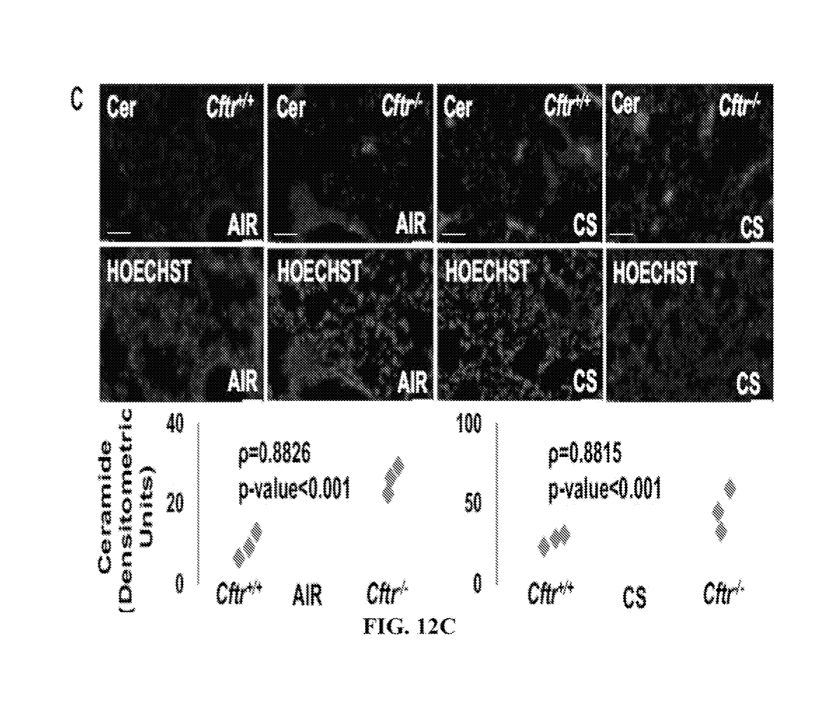

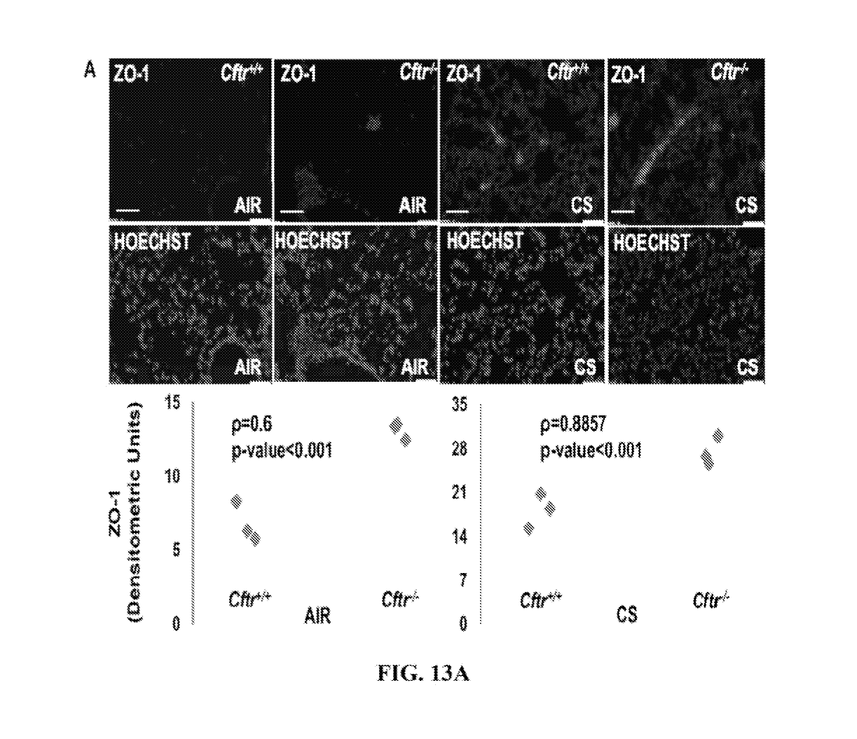

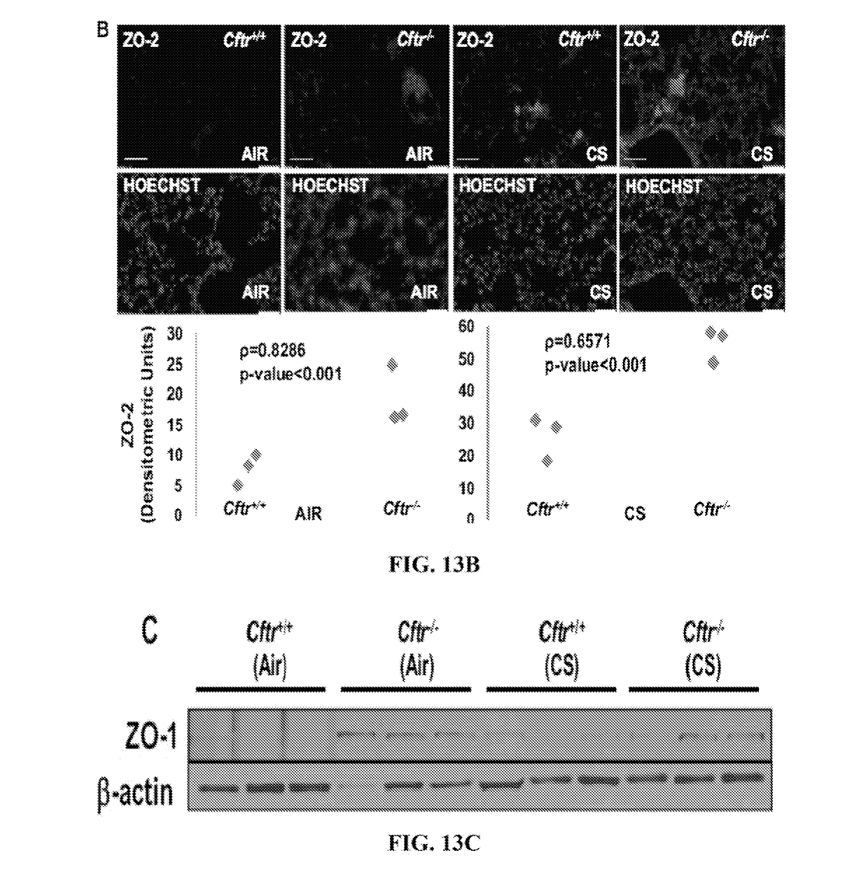

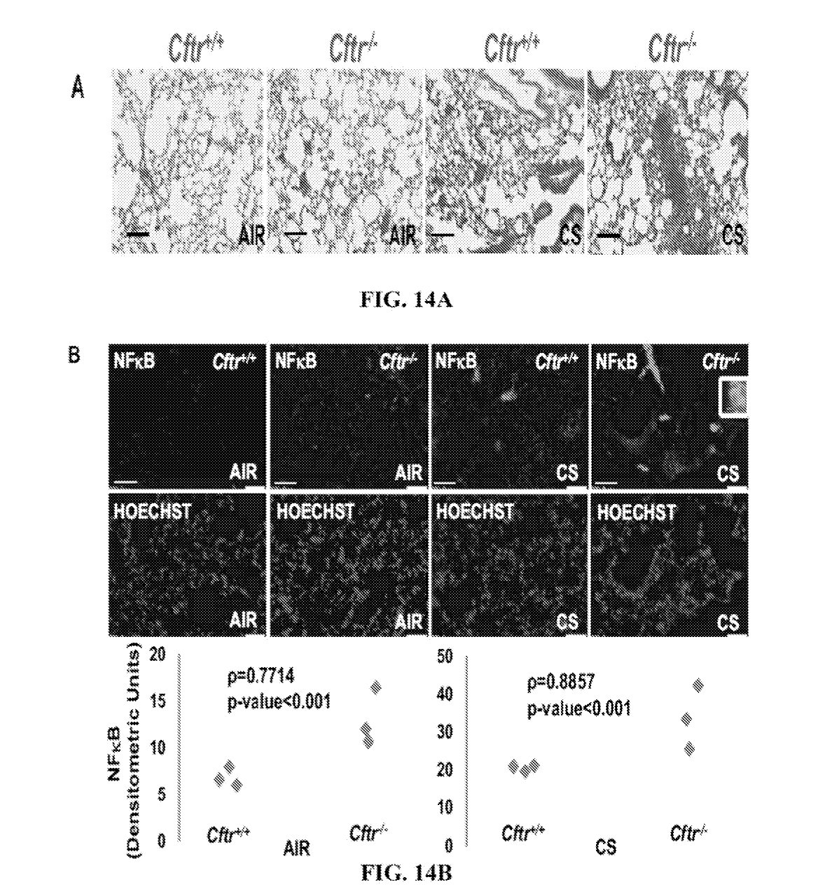

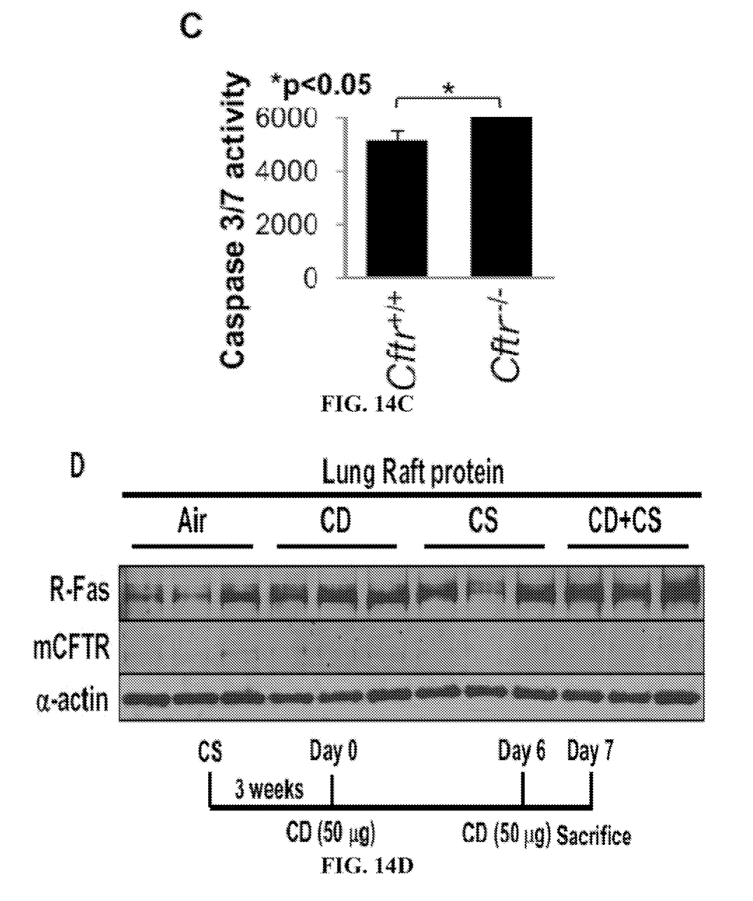

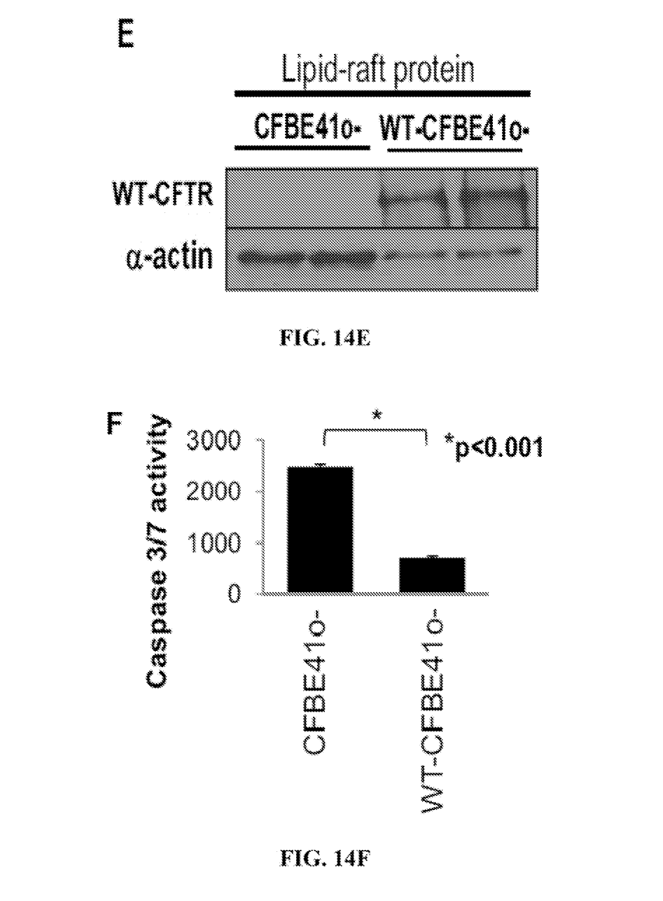

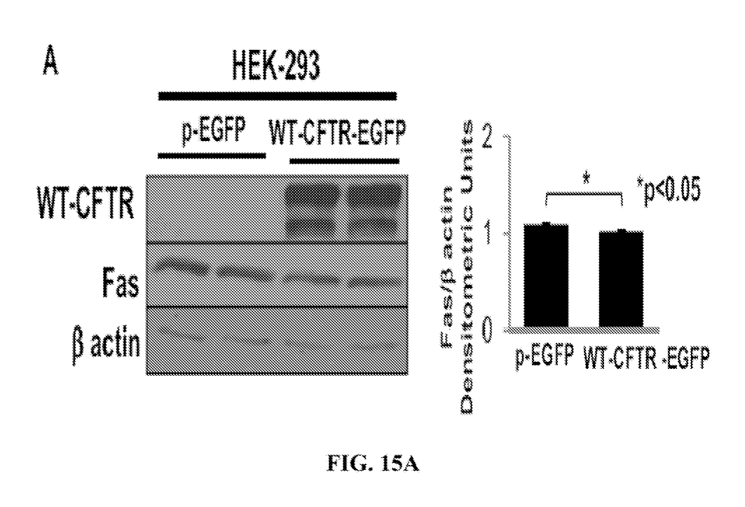

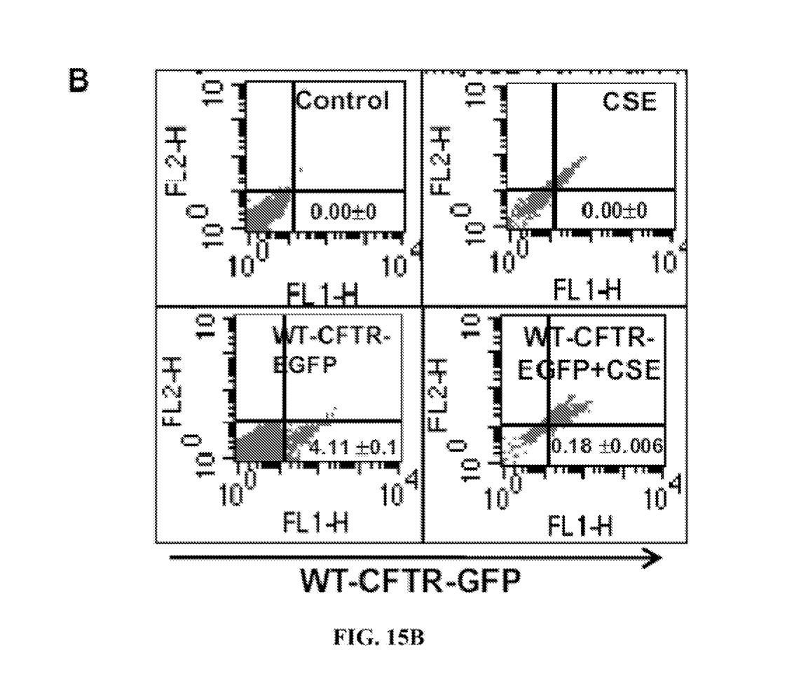

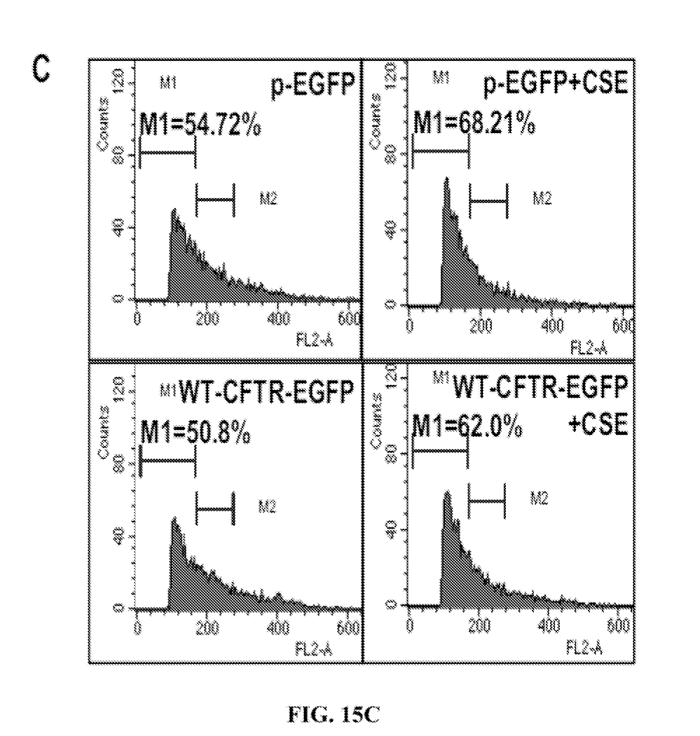

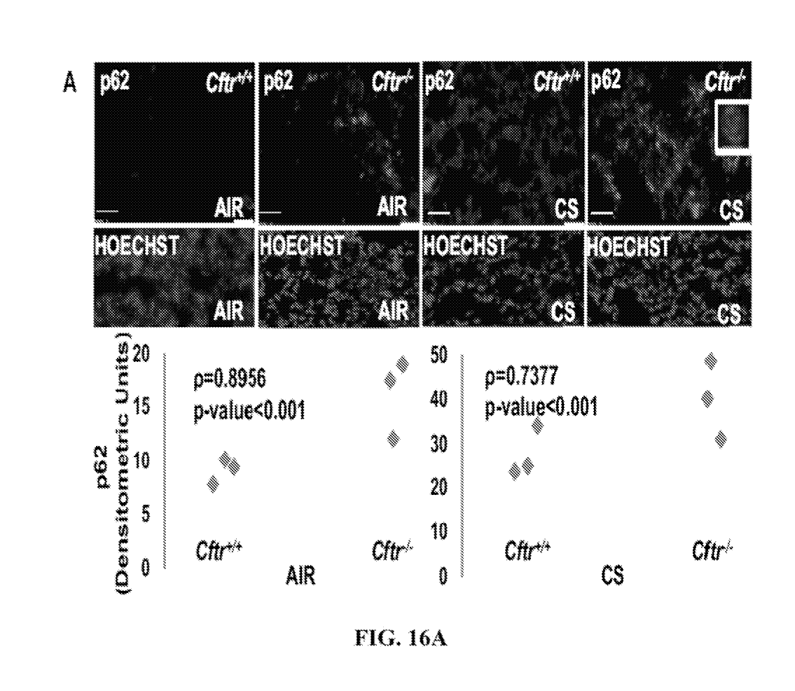

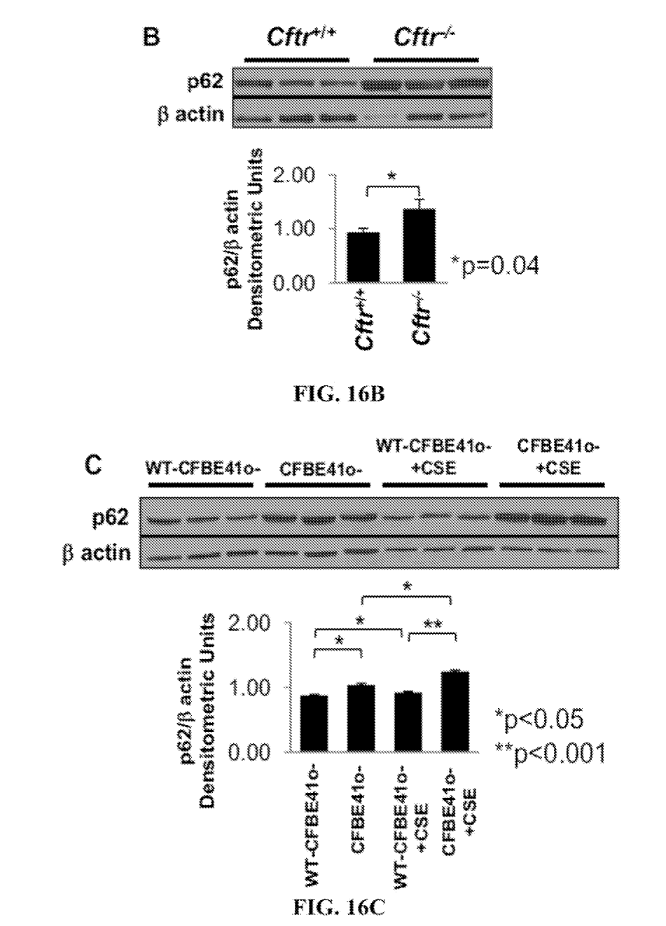

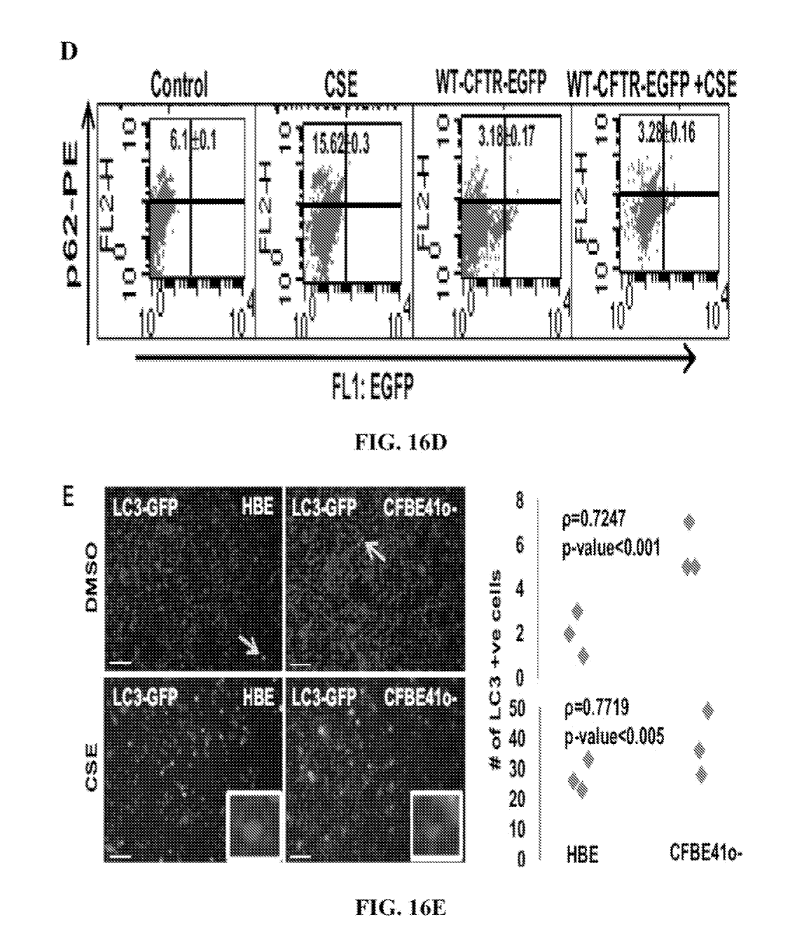

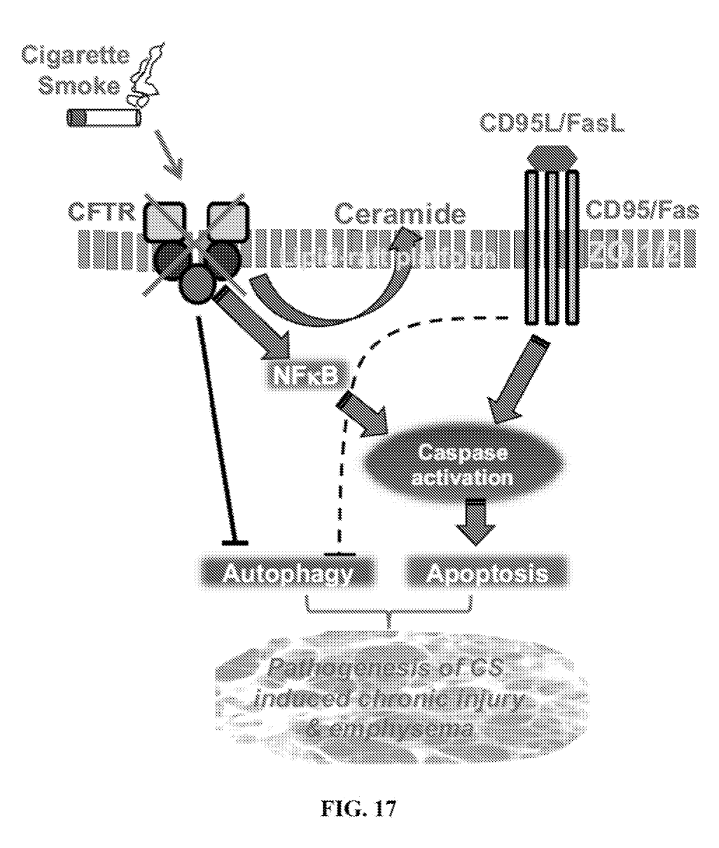

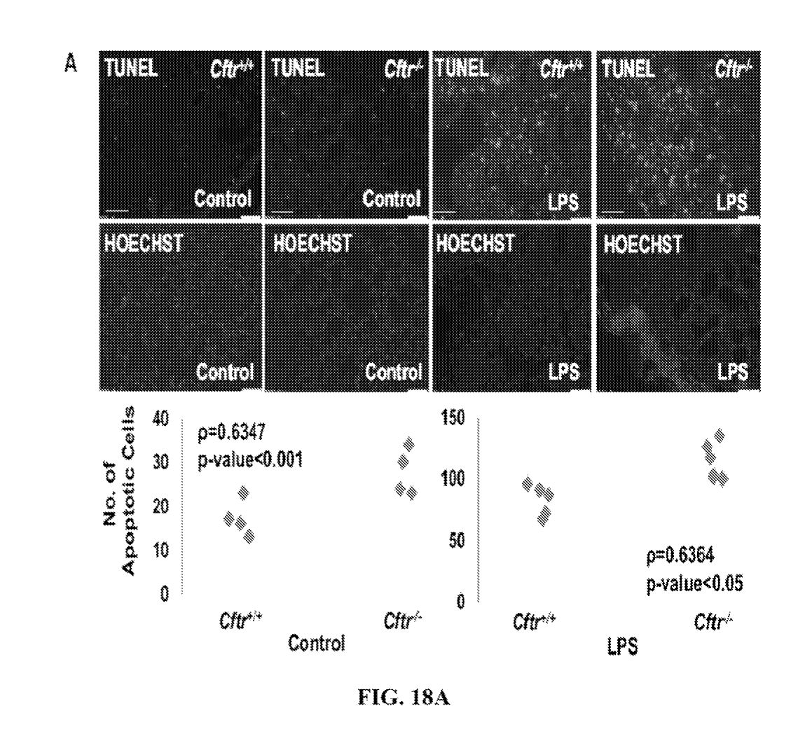

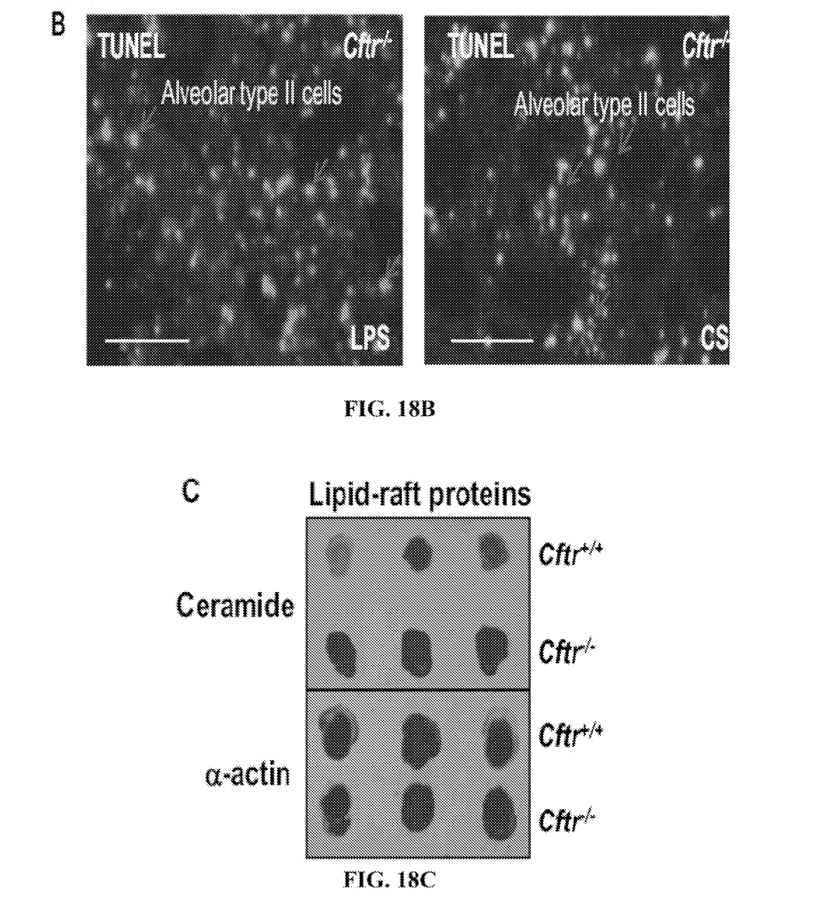

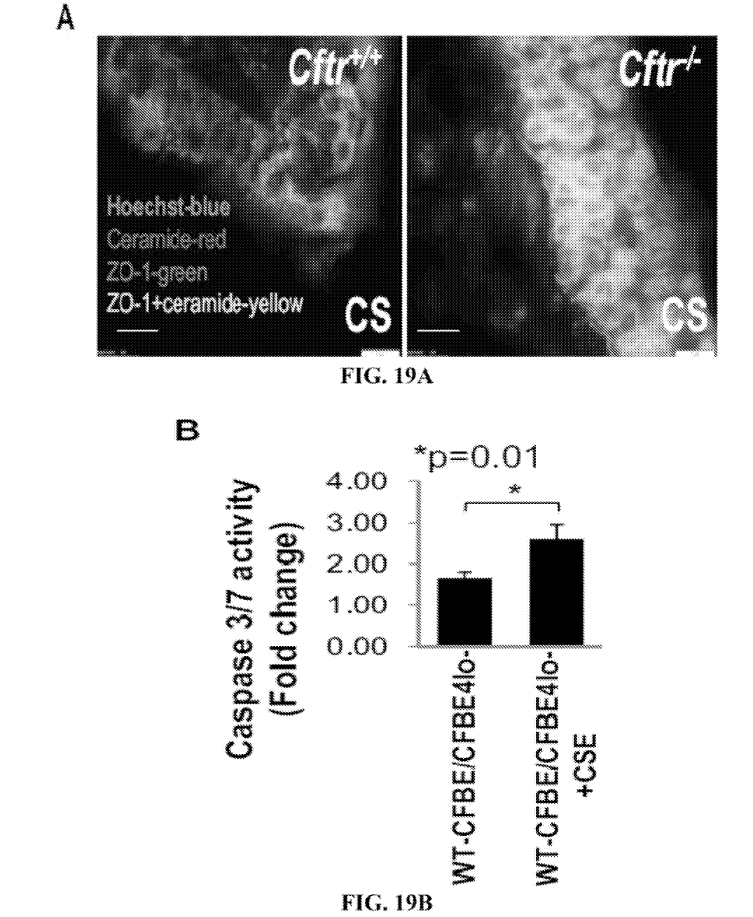

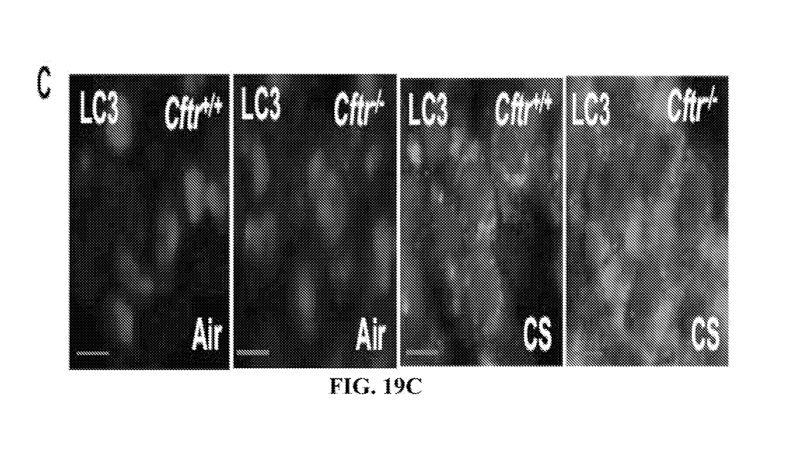

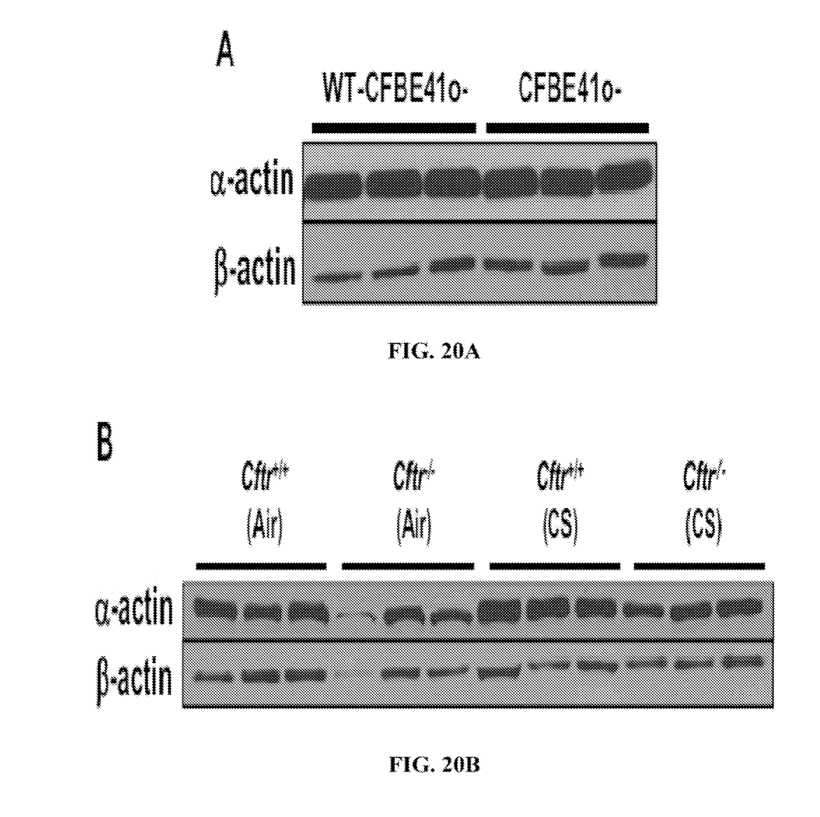

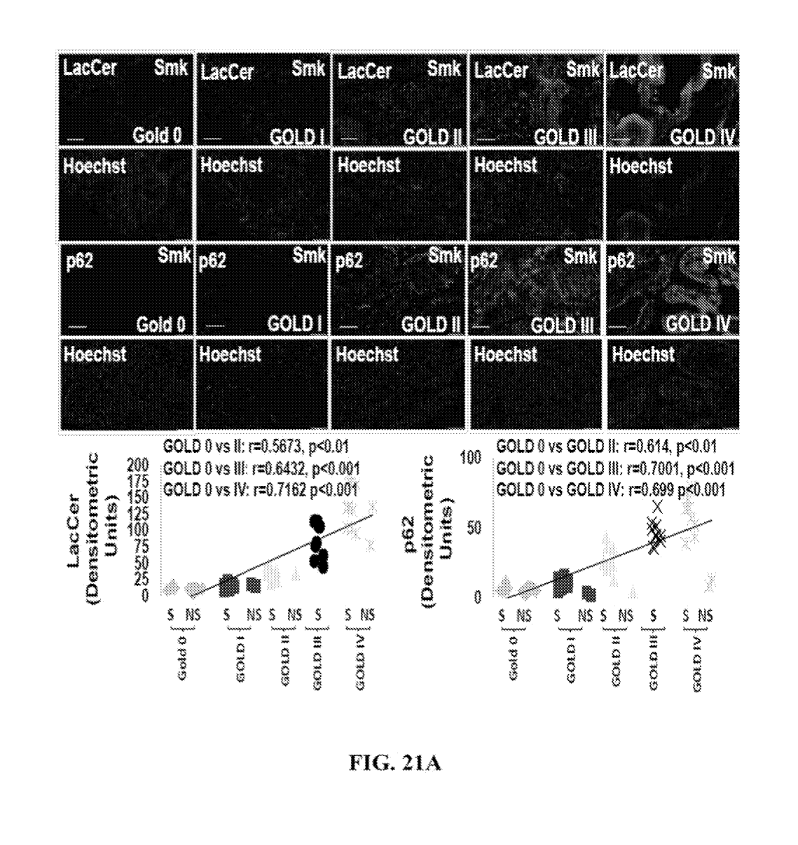

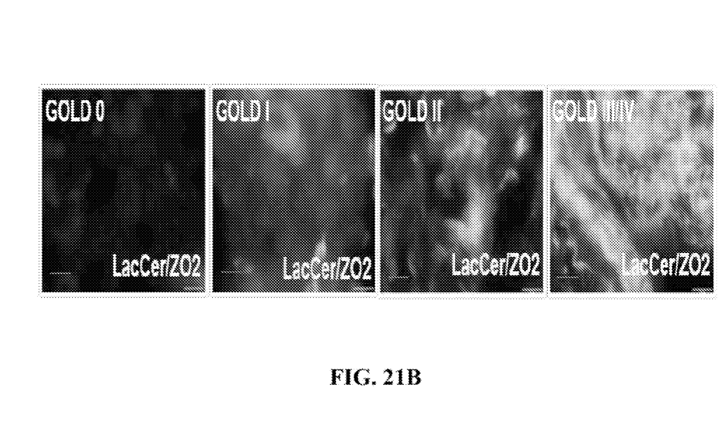

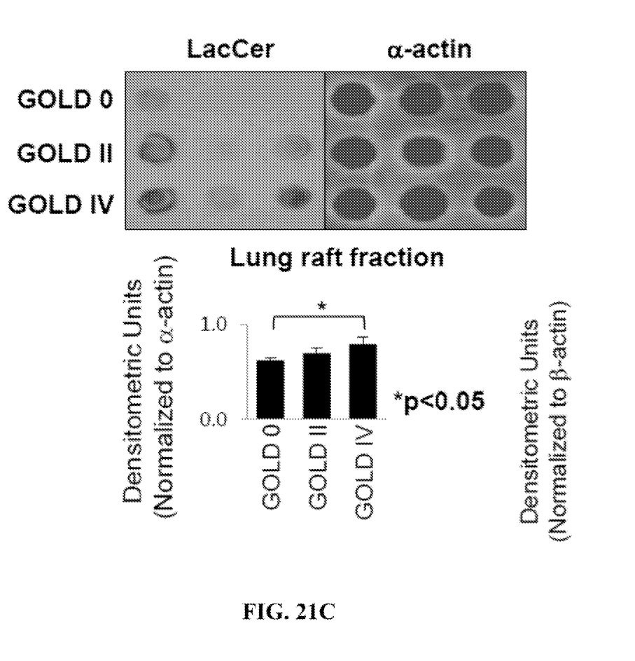

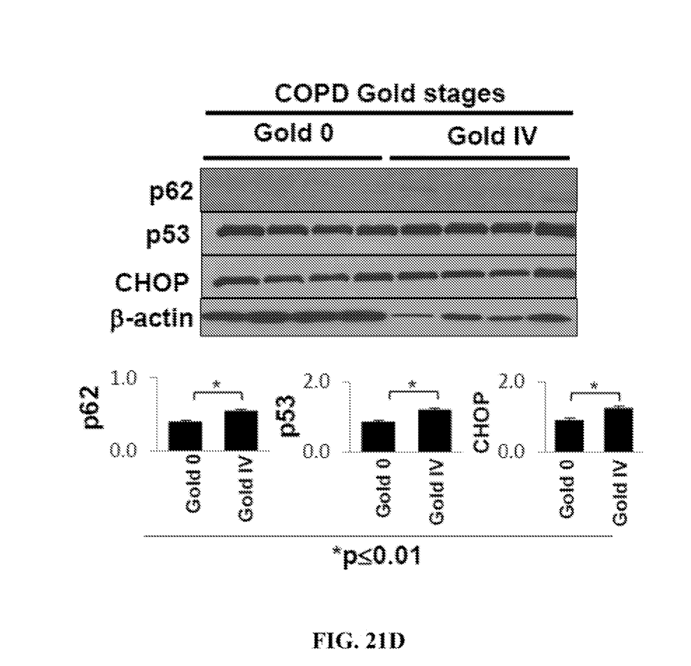

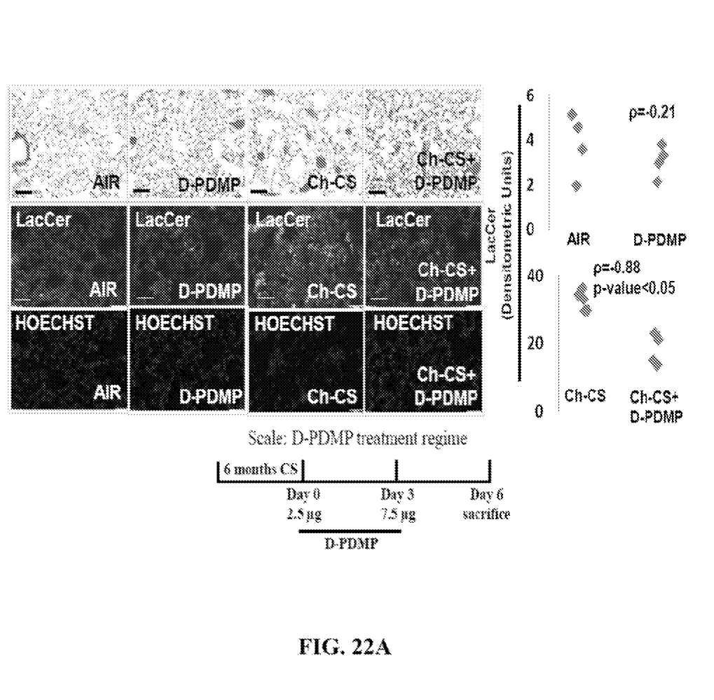

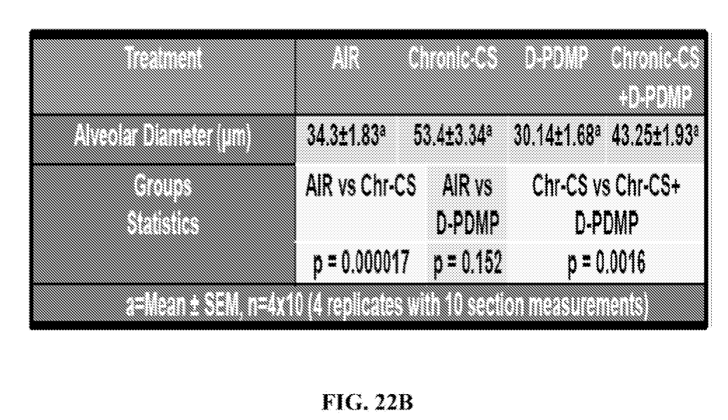

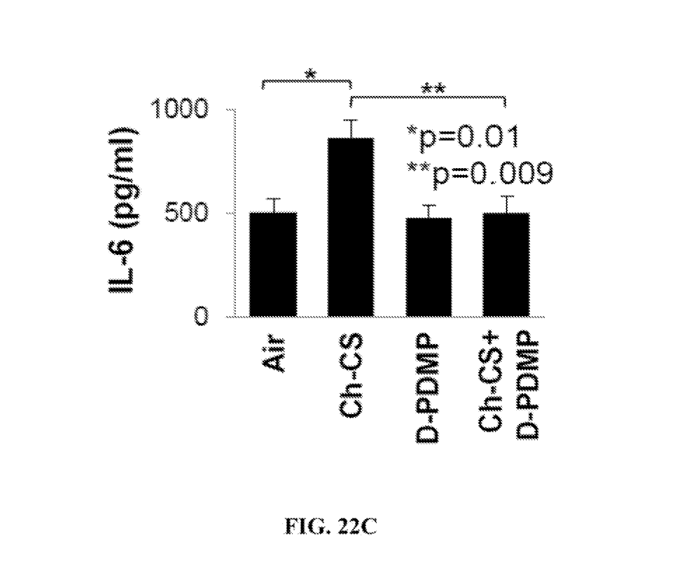

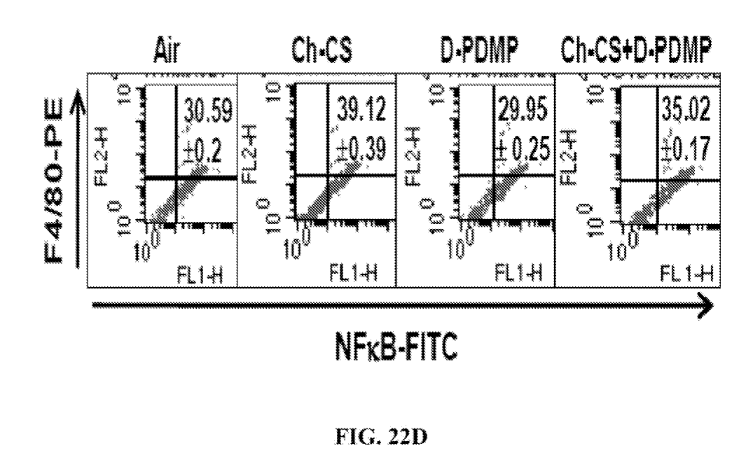

Our data demonstrate the importance of cell surface and lipid-raft CFTR in regulating ceramide-mediated inflammatory signaling. The C-terminal PDZ-interacting domain of CFTR protein is crucial for its apical membrane polarization and functional robustness (24, 25). To investigate the role of this domain in CFTR-dependent inflammatory responses, we overexpressed WT- or ΔTRL- (CFTR lacking the PDZ binding domain) CFTR-GFP in HEK-293 cells and quantified ceramide levels by flow cytometry. We found that expression of ΔTRL-CFTR triggers higher ceramide accumulation (FIG. 6A, upper panel), which is more prominent upon CSE treatment (FIG. 6A, lower panel). Expression of ΔTRLCFTR also decreases the binding of E. coli LPS-Alexa Fluor 488 to the plasma membrane (FIG. 6B). Because CFTR has been described as a pattern recognition molecule for LPS binding (26), our data demonstrate that the PDZ binding domain of CFTR may be crucial for its function as a pattern recognition molecule. We also demonstrate that expression of ΔTRL-CFTR leads to less CFTR protein reaching the lipid-raft fraction (FIG. 6Ca, 6Cb [6Ca, 30-s exposure; 6Cb, 20-min exposure], 6D). Treatment with TNF-a induces the localization of CFTR to the lipid-rafts, but ΔTRLCFTR mutation compromises its translocation to lipid-raft. Our data suggest that PDZ binding domain is required for CFTR membrane stability, and lipid-raft-localization and signaling (FIG. 7). We anticipate binding to PDZ domain-containing proteins (ZO-1/2) may be critical for this process.

### Critical Role of CFTR Dependent Lipid-Rafts in Cigarette Smoke Induced Lung Epithelial Injury

### Materials and Methods

Reagents and Treatments.

The CFBE41o-, CFBE41o-WT-CTFR (from Dr. Dieter Gruenert), 16HBEo- and HEK-293 cells were cultured at 37° C. with 5% CO2 in MEM or DMEM-F12 media, supplemented with 10% Fetal Bovine Serum (FBS) and 1% Penicillin, Streptomycin and Amphotericin B (PSA) from Invitrogen. We used normal 16HBEo- and CFBE41o-cells for GFP-LC3 reporter assay, and the CFBE41o-WT-CTFR and CFBE41o-cells were used to evaluate whether the effects of CFTR deficiency on apoptosis induction can be reversed by stable WTCFTR expression on CFBE41o-cells. We verified the role of WT-CFTR in regulating apoptotic and autophagy responses using HEK-293 cells transiently transfected with WT-CFTR-EGFP and/or p-EGFP. For in vitro experiments, cells were treated with 200 μg/ml cigarette smoke extract (CSE, Murty Pharmaceuticals Inc) or DMSO vehicle for the indicated time points. The cells were transfected with EGFP-LC3B plasmid using Lipofectamine 2000 reagent (Invitrogen) as previously described (46) followed by CSE treatment and analyzed by immunofluorescence microscopy to detect and count GFP-LC3 positive cells. The HEK-293 cells were similarly transiently transfected with p-EGFP or WT-CFTR-EGFP plasmids and treated with CSE (200 μg/ml) or DMSO vehicle for 24 hours. The mice were exposed to CS (acute, 5 days or subchronic, 4 weeks) or Pseudomonas aeruginosa LPS (Pa-LPS, Sigma) as described (4).

Murine Experiments.

All animal experiments were carried out in accordance with the Johns Hopkins University (JHU) Animal Care and Use Committee (ACUC) approved protocols. We used age, weight and sex matched (8 weeks old), B6-12956-Cftr^{-/-}(Cftrtm1Kthc-TgN(FABPCFTR)) (41, 42) and Cftr^{+/+} inbredmice strains (procured from Case Western Reserve University Animal Resource Center, representative data shown for at least n=3 in all experiments). All mice were housed in controlled environment and pathogen-free conditions. The mice (3 mice per group, 8 weeks old) were exposed to CS using the TE-2 cigarette smoking machine (Teague Enterprises, Davis, Calif.). The CS was generated by burning research grade cigarettes (3R4F, 0.73 mg nicotine per cigarette) purchased from the Tobacco Research Institute (University of Kentucky, Lexington, Ky.) for 5 hours/day for 5 days (acute exposure) or 4-weeks (sub-chronic exposure). An average total particulate matter (TPM) of 150 mg/m3 was recorded in real time during the smoking protocols. The control group of mice was exposed to filtered room-air and all the mice were sacrificed 2 hours after the last CS exposure. To evaluate the effect of lipid-raft CFTR, we used our previously described method using cyclodextrin (CD) treatment under conditions known to disrupt lipid-raft and deplete CFTR (4). The sub-chronic CS exposed mice were treated intratracheally with CD (2×50 μg in PBS as vehicle, see scale in FIG. 3D) at indicated time points and sacrificed 2 hours after the last CS exposure. To verify the findings from CS exposure, we also used Pa-LPS induced lung injury model as recently described (4). Briefly, the Cftr^{+/+} and Cftr^{-/-} mice were treated with 20 μg/mouse Pa-LPS by intratracheal instillation for 24 hours. The lungs from CS exposed or Pa-LPS treated mice were harvested and fixed in 10% buffered formalin phosphate (Fisher Scientific), paraffin embedded and cut into longitudinal sections (5 micron thick) on glass slides for immunostainings or to detect the number of apoptotic cells by TUNEL assay.

Immunohistoloxy and TUNEL Assay.

The longitudinal tissue sections from murine lungs were immunostained with the primary antibodies (1-2 μg/ml) for ceramide (mouse monoclonal, Alexis Biochemical's), Fas (rabbit 7 polyclonal, Santa Cruz Biotechnology, scbt) NFκB (rabbit polyclonal, scbt), p62 (mouse monoclonal, BD Biosciences), Zona occludens-1 (ZO-1) (rabbit polyclonal, scbt)/ZO-2 (goat polyclonal, scbt) and LC-3β (goat polyclonal, scbt) followed by the secondary antibodies (1:200 dilution), using our previously described protocol (47). The secondary antibodies used were goat anti-rabbit IgG FITC (scbt, 1 μg/ml), donkey anti-mouse IgG Alexa Fluor 594 (Invitrogen, 10 μg/ml) and donkey anti-goat Dylight 594 (Jackson ImmunoResearch, 1.25 μg/ml). Nuclei were detected by Hoechst (Invitrogen, 2 μg/ml) staining while H&E was used to evaluate lung morphology and inflammatory state. Images were captured by Axiovert 200 Carl Zeiss Fluorescence microscope using the Zeiss Axiocam HRC camera and Axiovision software. The number of apoptotic cells in longitudinal lung sections from Cftr^{+/+} and Cftr^{-/-} mice exposed to CS or Pa-LPS were quantified by DeadEnd™ Fluorometric terminal deoxynucleotidyl transferase (TdT) mediated dUTP nick end labeling (TUNEL) kit (Promega).

Autophagy Reporter Assay.

The 16HBEo- and CFBE41o cells were transiently transfected with EGFP-LC3B plasmid (vector backbone: pEGFP-C3, Addgene) for a total of 48 hours. The cells were treated with 200 μg/ml CSE for the last 24 hours, and analyzed by immunofluorescence microscopy (4) using Axiovert 200 Carl Zeiss Fluorescence microscope, Zeiss Axiocam HRC camera and Axiovision software as described above. The peri-nuclear localization of GFP-LC3 was determined at 100× magnification. The number of GFP-LC3-positive peri-nuclear aggregates was counted in each well and the representative data of triplicate samples is shown. 8

Caspase-3/7 Assay.

The CFBE41o- and CFBE41o-WT-CTFR cells were cultured as previously described (4) and 104 cells/well were plated in a 96 well flat bottom tissue culture plate (100 μl volume/well). Cells were treated with 200 μg/ml CSE for 24 hours or equal volume of DMSO vehicle as a control. The caspase-3/7 activity was quantified using Promega's Caspase-Glo™ 3/7 Assay as described before (47). Briefly, equal volume of the caspase-3/7 reagent was added to each well and the plate was incubated for 30-60 minutes in the dark. The luminescence was measured and the fold change in caspase activity was calculated.

Flow Cytometry.

The HEK-293 cells were transiently transfected with p-EGFP or WT-CFTR-EGFP plasmid and treated with DMSO vehicle or CSE (200 μg/ml) for 24 hours. The cells were either analyzed for WT-CFTR-EGFP expression or the number of p62-positive cells, using the BD FACS Caliber flow cytometer as previously described (4). Next we quantified percentage of apoptotic cells (in M1-phase) by propidium iodide (Sigma) staining as recently described (33). Briefly, cells were treated as above and fixed with ice cold 70% ethanol followed by propidium iodide (1 mg/ml) staining and flow cytometry (33). The cells in the M1 phase were gated to quantify the statistical change in percentage of apoptotic cells.

Immunoblotting of Total and Lipid-Raft Proteins.

The total lung lysates isolated as before (4, 5, 23) from Cftr^{+/+} and Cftr^{-/-} mice (air or CS exposed) were run on 10% SDS PAGE and analyzed for the changes in the expression of apoptotic marker, Fas (scbt), aggresome/defective-autophagy marker, p62 (BD), lipid-raft 9 marker, ZO-1 (scbt) and CFTR (rabbit polyclonal, scbt) by immunoblotting. The lysates from CFBE41o- and CFBE41o-WT-CTFR (DMSO or CSE treated) or HEK-293 cells (pEGFP or WTCFTR— EGFP) were similarly analyzed for p62 (BD), Fas (scbt) or CFTR [(scbt), 596 (CF Foundation & UNC under MTA)] expression. The blots were re-probed for α- or β-actin (Sigma) as the loading control. The lipid-raft protein fraction was isolated from cells or murine lungs (air, sub-chronic-CS and/or CD) as we recently described (4) and immunoblotted for Fas (scbt), CFTR (scbt, mice and 596-ab, cells) and α-actin (Sigma). We also isolated lipid-raft protein from Cftr^{+/+} and Cftr^{-/-} mice lungs and spotted (20 μg, dot blot) them on a 0.45 μm nitrocellulose membrane followed by immunoblotting for ceramide (Alexis) and α-actin.

Statistical Analysis.

Data are represented as the mean±SEM (SD for flow cytometry analysis) of at least three experiments. Student t test and ANOVA were used to determine the statistical significance. The microscopy data were analyzed by densitometry (MATLAB R2009b; Mathworks, Natick, Mass.) followed by Spearman's correlation coefficient analysis to calculate the significance among the indicated groups.

### Example 1

### CFTR Controls Alveolar Cell Apoptosis in CS Induced Lung Injury

Alveolar cell apoptosis is the primary cause of cigarette smoke (CS) induced emphysema development (8). We have previously shown that lipid-raft localized CFTR regulates inflammatory signaling via ceramide in COPD subjects with emphysema and CS induced mice (4). In order to elucidate the role of CFTR in lung cell apoptosis, we quantified the number of 10 apoptotic cells in the lungs of Cftr^{+/+} and Cftr^{-/-} mice exposed to either room-air or acute CS by TUNEL assay. We found a significant constitutive increase in TUNEL positive apoptotic cells (predominantly alveolar type II) in the Cftr^{-/-} mice lungs (p<0.01) compared to the Cftr^{+/+}, that was further aggravated by acute CS exposure (FIG. 1A & Supplementary FIGS. 1A&B). The expression and lipid-raft clustering of CD95/Fas receptor is essential for activation of apoptotic signaling (15). We found a significant increase in Fas expression in the Cftr^{-/-} mice lungs compared to Cftr^{+/+} (FIG. 1B, p<0.001) that also correlates with a substantial increase in lung ceramide accumulation (FIG. 1C, p<0.001). The expression of Fas (apoptosis) and ceramide (apoptosis and inflammation) was further elevated in the CS exposed Cftr^{-/-} mice lungs compared to the Cftr^{+/+} (FIGS. 1B&C, upper and lower panels, p<0.001) indicating that CFTR deficiency mediates CS induced lung injury by inducing apoptotic cell death. We were able to verify Fas accumulation in lipid-rafts (discussed below for FIG. 3D). We also confirmed this finding in the Pa-LPS induced lung injury model and found that Cftr^{-/-} mice have higher number of TUNEL positive apoptotic cells compared to Cftr^{+/+} (Supplementary FIGS. 1A&B, p<0.05). The data demonstrates that CFTR controls ceramide accumulation and alveolar cell apoptosis in CS induced lung injury.

### Example 2

### CFTR Regulates the Expression of Lipid-Raft Markers in the Murine Lungs

We have previously shown that CFTR negatively regulates lipid-raft clustering and the absence of CFTR leads to ceramide accumulation in lipid-rafts (4). We evaluated here the correlation of increased expression of lipid-raft markers, ZO-1 and ZO-2 with the changes in Fas- and ceramide-expression in the Cftr^{-/-} mice lungs. Since Fas receptor clustering to ceramide enriched raft-platforms is essential to trigger the apoptotic cascade (14, 15), we hypothesized that CFTR controls the clustering of Fas in lipid-rafts to regulate apoptotic signaling. To confirm this, we 11 analyzed the protein expression of ZO-1 and ZO-2 in the longitudinal lung sections of Cftr^{+/+} and Cftr^{-/-} mice exposed to acute CS by immunostaining. The data indicates significant upregulation of both constitutive and CS induced ZO-1/2 expression in the Cftr^{-/-} mice lungs (FIG. 2A, B, p<0.001). We verified this by immunoblotting total protein extracts of murine lungs from the same groups of mice that clearly show constitutive differences between Cftr^{+/+} and Cftr^{-/-}, ZO-1 expression (FIG. 2C). Although we did not see a significant change in CS induced ZO-1 expression in Cftr^{+/+} and Cftr^{-/-} mice lungs. It may be possible that ZO-1 localizes/clusters in the lipid-raft (insoluble protein fraction) upon CS exposure. Since we used soluble total protein extracts, we may not have detected the upregulation of ZO-1 upon CS exposure (as seen in FIG. 2A by immunostaining). Further studies are required in the purified lipid-raft fractions from the lungs of these mice to verify the data. Moreover, we also found a significant increase in ZO-1/ceramide co-staining in the CS exposed Cftr^{-/-} mice compared to the Cftr^{+/+} (Supplementary FIG. 2A). This data suggests that absence of lipid-raft CFTR leads to ceramide accumulation and raft-clustering that may induce the formation of signaling platforms involved in Fas receptor clustering and activation of apoptosis pathway.

### Example 3

### CFTR Controls CS Induced Inflammation and Apoptosis in the Murine Lungs

Acute CS exposure triggers NFκB mediated inflammatory signaling in the murine lungs (51). In order to verify if absence of CFTR aggravates CS mediated NFκB activation and inflammation, we used the Cftr^{-/-} murine model and stained the longitudinal lung sections from CS exposed Cftr^{+/+} and Cftr^{-/-} mice with hematoxylin & eosin (H&E) and NFκB (using an antibody). We found that Cftr^{-/-} mice show a significant constitutive increase in inflammation, NFκB activation 12 and nuclear localization compared to Cftr^{+/+} (p<0.001, FIG. 3A, B). Acute CS exposure enhanced the inflammation, NFκB levels and nuclear localization (FIG. 3B, inset, upper right panel) in the Cftr^{-/-} mice lungs compared to the Cftr^{+/+} indicating that CFTR regulates inflammatory signaling in response to CS exposure through NFκB activation. We also verified that Cftr^{-/-} mice lungs have constitutively higher caspase-3/7 activity as compared to Cftr^{+/+} (FIG. 3C). Next, to evaluate if lipid-raft CFTR regulates Fas mediated apoptosis, we treated Cftr^{+/+} mice with methyl-β-cyclodextrin (CD) (4, 46) and/or sub-chronic CS. We observed that both CD and CS treatment increases lipid-raft Fas expression while CFTR levels decrease (FIG. 3D). Co-treatment with CD and CS had a synergistic effect. We also verified this using the cell lines and found that stable WT-CFTR expression in CFBE41o-cells (CFBE41o-WT-CTFR cells) controls the (−1.6 fold, p<0.001) constitutively elevated caspase-3/7 activity in CFBE41o-cells (FIGS. 3E & F). Our data indicate that lipid-raft-CFTR controls alveolar cell apoptosis (FIGS. 1&2) by regulating membrane-ceramide accumulation (FIG. 1 & Supplementary FIGS. 1C, 2A&B) as a mechanism to induce Fas-receptor clustering and caspase-3/7 activity (FIG. 3). Since intratracheal instillation of active caspase-3 or ceramide induces emphysema like-phenotype in the murine emphysema model (49), the observed decrease in CS induced membrane-CFTR and subsequent ceramide accumulation contributes to the pathogenesis of severe emphysema as recently demonstrated by our group (4).

### Example 4

### WT-CFTR Controls Cigarette Smoke Extract Induced Apoptosis

In order to confirm our hypothesis that WT-CFTR controls Fas mediated apoptotic signaling, we over-expressed WT-CFTR in HEK-293 cells and found a significant decrease in constitutive Fas expression (FIG. 4A, p<0.05). We also found that the WT-CFTR expression in HEK-293 cells is significantly downregulated by cigarette smoke extract (CSE) treatment (FIG. 4B, lower panel, 13 p<0.001) supporting our recent data (4). Next, we verified that WT-CFTR expression controls CSE induced apoptosis as seen by decrease in number of cells in the M1-phase (FIG. 4C). The data indicate that WT-CFTR regulates Fas expression and CSE induced apoptosis.

### Example 5

### CFTR Augments CS Induced Autophagy Response

Autophagy is a critical cellular homeostatic process that disposes damaged protein aggregates (aggresomes) that are associated with several chronic inflammatory diseases and cancer (9, 34). A recent study by Luciani et al (19) clearly demonstrate a critical role of CFTR in maintaining the robust autophagic machinery, and defective CFTR results in inhibition of autophagy leading to an inflammatory outcome. We analyzed the accumulation of p62 (defective autophagy marker) in the longitudinal lung sections of room-air and acute-CS-exposed Cftr^{+/+} and Cftr^{-/-} mice. We demonstrate here a significant upregulation (p<0.05) of p62 expression in the Cftr^{-/-} mice lungs compared to the Cftr^{+/+} (FIGS. 5A & B). Acute CS exposure induces p62 expression and peri-nuclear accumulation (FIG. 5A, inset, upper right panel) in murine lungs that was significantly higher (p<0.001) in Cftr^{-/-} mice compared to Cftr^{+/+}. We further evaluated this hypothesis in CS-extract exposed human cell lines-CFBE41o-WT-CTFR cells/CFBE41o-'s. We demonstrate that intrinsically higher p62 expression in CFBE41o-cells can be corrected by stable WT-CFTR expression in CFBE41o-WT-CTFR cells (FIG. 5C, p<0.05). Moreover, treatment of these cells with CSE further induces p62 expression but it's significantly lower in presence of WT-CFTR (FIG. 5C, p<0.001). We verified this observation in CSE treated HEK-293 cells overexpressing WT-CFTR and observed a significant decrease in CSE induced p62-positive cells with CFTR expression (FIG. 5D). The accumulation of LC3-punctate (peri-nuclear aggregates) is considered a reliable marker of autophagy (19). We found a significant increase in peri-nuclear accumulation of GFP-LC3 in CFBE41o-cells compared to 16HBEo- (p<0.001, FIG. 5E, upper 14 panel) that was markedly induced by CSE treatment (p<0.005, FIG. 5E, lower panel). Moreover, we also found an increase in constitutive as well as acute CS induced LC3 expression in the Cftr−/− mice lungs compared to Cftr^{+/+}(Supplementary FIG. 2C). We not only confirmed here the findings of Luciani et al but also demonstrate the critical role of CFTR in regulating CS induced autophagy response. The absence of CFTR results in a significant increase in p62-positive perinuclear aggregates (aggresomes) that may explain unrelenting inflammation and apoptosis. Our data demonstrates a novel mechanism by which CFTR protects the alveolar epithelial cells from CS induced aggresome formation and resulting inflammatory/apoptotic phenotype.

### Critical Role of Lactosylceramide-Accumulation in Pathogenesis of Lung Injury and Emphysema

The present invention is also directed to downstream metabolites of ceramide as a therapeutic target and diagnostic/progonistic tool for subject with pulmonary conditions. As described herein, the inventors have elucidated the role of the metabolite Lactosylceramide (LacCer) in pathogenesis of lung injury and emphysema. In addition to ceramide, its downstream metabolite LacCer accumulates in severe emphysema. Moreover, LacCer accumulation correlates with severity of emphysema and expression of lipid-raft (ZO-2) and defective-autophagy (p62) markers. Pa-LPS treatment and cigarette smoke (CS) induces LacCer-accumulation in murine lungs that correlates with increased p62 expression and NFκB mediated neutrophil (NIMP-R14) chemotaxis. LacCer-inhibitors significantly (p<0.05) decrease NFκB, p62 and NIMP-R14 expression in the murine lungs and IL-6, caspase-3/7 and MPO activities in BALFs of Pa-LPS/CS exposed mice. The CS extract treated in vitro (Raw264.7/Beas2b) model was used to further confirm that CS induces LacCer-accumulation that plays a critical role in triggering aberrant-autophagy (p62), apoptosis (caspase-3/7) and inflammation (NFκB), hence emphysema pathogenesis, that can be controlled by LacCer inhibitors (p<0.001). Thus, LacCer plays a critical role in the pathogenesis of lung injury and emphysema and selective LacCer-inhibition has therapeutic potential in treating chronic lung disease.

1. A method for assessing the severity of lung damage from a pulmonary condition in a subject comprising the steps of:
a. obtaining a sample from the subject;
b. measuring the level and/or functional activity of membrane/lipid-raft cystic fibrosis transmembrane conductance regulator (CFTR) in the sample obtained from the subject;
c. measuring the level of ceramide or its species in a sample from the subject; and
d. comparing the membrane/lipid-raft CFTR level and/or functional activity and level of ceramide or its species to a control sample;
wherein a difference in membrane/lipid-raft CFTR level and/or functional activity and level of ceramide or its species is indicative of the severity of lung damage.

2. The method of claim 1, further comprising treating the subject based on the severity of lung damage.

3. The method of claim 2, wherein the treatment comprises administering an effective amount of an agent that inhibits the synthesis of ceramide or its species.

4. The method of claim 2, wherein the treatment comprises administering an effective amount of a CFTR agonist.

5. The method of claim 1, wherein pulmonary condition is selected from the group consisting of chronic obstructive pulmonary disease (COPD), emphysema, cystic fibrosis, Pseudomonas aeruginosa bacterial infection, or biomass/cigarette-smoke exposure.

6. The method of claim 1, wherein the pulmonary condition is COPD.

7. The method of claim 1, wherein the pulmonary condition is emphysema.

8. The method of claim 1, wherein the pulmonary condition is cystic fibrosis.

9. The method of claim 1, wherein the subject is a smoker or is exposed to biomass smoke.

10. The method of claim 1, wherein the ceramide species is lactosylceramide.

11. A method for predicting risk of lung damage from a pulmonary condition in a subject comprising the steps of:
a. obtaining a sample from the subject;
b. measuring the level and/or functional activity of membrane/lipid-raft CFTR in the sample obtained from the subject;
c. measuring the level of ceramide or its species in the sample obtained from the subject; and
d. comparing the membrane/lipid-raft CFTR level and/or functional activity and level of ceramide or its species to a control sample;
wherein a difference in membrane/lipid-raft CFTR level and/or functional activity and level of ceramide or its species is indicative of a risk of lung damage.

12. The method of claim 11, further comprising treating the subject based on the risk of lung damage.

13. The method of claim 12, wherein the treatment comprises administering an effective amount of an agent that inhibits the synthesis of ceramide or its species.

14. The method of claim 12, wherein the treatment comprises administering an effective amount of a CFTR agonist.

15. The method of claim 11, wherein pulmonary condition is selected from the group consisting of chronic obstructive pulmonary disease (COPD), emphysema, cystic fibrosis, and Pseudomonas aeruginosa bacterial infection or biomass/cigarette-smoke exposure.

16. The method of claim 11, wherein the pulmonary condition is COPD.

17. The method of claim 11, wherein the pulmonary condition is emphysema.

18. The method of claim 11, wherein the pulmonary condition is cystic fibrosis.

19. The method of claim 11, wherein the subject is a smoker or is exposed to biomass smoke.

20. The method of claim 11, wherein the ceramide species is lactosylceramide.

21. A method for treating a pulmonary condition in a subject comprising the steps of:
a. obtaining a sample from the subject;
b. measuring the level and/or functional activity of membrane/lipid-raft CFTR in the sample obtained from the subject;
c. measuring the level of ceramide or its species in the sample obtained from the subject;
d. comparing the membrane/lipid-raft CFTR level and/or functional activity and level of ceramide or its species to a control sample; and
e. administering an effective amount of a ceramide inhibitor and/or a CFTR agonist based on the membrane/lipid-raft CFTR level and/or functional activity and level of ceramide or its species.

22. The method of claim 21, wherein the ceramide inhibitor inhibits one or more of the enzymes involved in the synthesis of ceramide or its species.

23. A method for treating a pulmonary condition in a subject comprising the step of administering a therapeutically effective amount of at least one agent that inhibits the synthesis of ceramide or its species.

24. The method of claim 21, wherein the ceramide species is lactosylceramide.

25. A method for treating a pulmonary condition in a subject comprising the step of administering a therapeutically effective amount of at least one CFTR agonist that increases membrane/lipid-raft level and/or functional activity.

26. The method of claim 21, wherein the pulmonary condition is selected from the group consisting of chronic obstructive pulmonary disease (COPD), emphysema, cystic fibrosis, and Pseudomonas aeruginosa bacterial infection or biomass/cigarette-smoke exposure.

27. The method of claim 21, wherein the pulmonary condition is COPD.

28. The method of claim 21, wherein the pulmonary condition is emphysema.

29. The method of claim 21, wherein the pulmonary condition is cystic fibrosis.

30. The method of claim of claim 21, wherein the subject is a smoker or is exposed to biomass smoke.

31. The method of claim 22, wherein the enzyme is selected from the group consisting of serine palmitoyltransferase, 3-ketosphinganine reductase, dihydroceramide synthase, dihydroceramide desaturase, sphingomyelinase, ceramide synthase, and lactosylceramide synthase.

32. The method of claim 23, wherein the at least one agent is an antisense molecule.

33. The method of claim 23, wherein the at least one agent is an RNA interference agent.

34. The method of claim 23, wherein the at least one agent is an miRNA.
